(19) United States
(12) Patent Application Publication
(10) Pub. No.: US 2025/0135002 A1
(43) Pub. Date: May 1, 2025

(54) COMPOSITIONS FOR AND METHOD OF EFFECTING TUMOR CELL DEATH

(71) Applicant: Duke University, Durham, NC (US)

(72) Inventors: Chuan-Yuan Li, Durham, NC (US); Fang Li, Durham, NC (US)

(21) Appl. No.: 18/835,496

(22) PCT Filed: Feb. 3, 2023
(86) PCT No.: PCT/US2023/061927

§ 371 Date: Aug. 2, 2024

Related U.S. Application Data
(60) Provisional application No. 63/306,634, filed on Feb. 4, 2022.

(51) Int. Cl. A61K 40/41; A61K 38/00; A61K 40/11; A61K 40/31; A61K 45/06; A61P 35/00; C07K 14/47; C07K 14/705; C07K 14/71; C07K 14/725; C07K 16/18; C12N 5/0783

(52) U.S. Cl. CPC A61K 40/4285; A61K 40/11; A61K 40/31; A61K 45/06; A61P 35/00; C07K 14/47; C07K 14/7051; C07K 14/70517; C07K 14/70521; C07K 14/71; C07K 16/18; C12N 5/0636; A61K 38/00; C07K 2319/02; C07K 2319/03; C07K 2319/40

(57) ABSTRACT

Disclosed herein are compositions comprising a chimeric antigen receptor targeting phosphatidylserine on the surface of cancer cells and methods of using the compositions to treat a cancer in a subject.

### CROSS-REFERENCE TO RELATED APPLICATIONS

This application claims priority to U.S. Provisional Application No. 63/306,634 filed 4 Feb. 2022, which is incorporated herein in its entirety.

### STATEMENT REGARDING FEDERALLY SPONSORED RESEARCH OR DEVELOPMENT

This invention was made with government support under CA216876 awarded by the National Institutes of Health. The government has certain rights in the invention.

### REFERENCE TO THE SEQUENCE LISTING

The Sequence Listing submitted 3 Feb. 2023 as an XML file named “23-2064-WO_Sequence-Listing”, created on 3 Feb. 2023 and having a size of 1 MB is hereby incorporated by reference pursuant to 37 C.F.R. § 1.52 (e) (5).

### BACKGROUND

In cancer treatment, it is highly desirable to exploit cancer-specific markers for therapeutic development. Many such markers are cell surface markers, which provide excellent targets to develop cell-based therapy such as CAR-T, CAR-NK, and CAR-Macrophage.

Phosphatidylserine (PS) is an abundant lipid molecule and an integral part of the cellular membrane. Unlike other abundant lipid molecules that evenly distribute in both the outer and inner cellular membranes, phosphatidylserine normally only resides in the inner cellular membrane. PS often “flips” from the inner to the outer cellular membrane in dying cells, especially in apoptotic cells. As a result, cell surface levels of PS have become the most widely used molecular marker to quantify cellular apoptosis in research. Most of the current paradigms for cancer therapy assume that PS-expressing tumor cells, either from exposure to internal or external stressors, are destined to die from apoptosis. However, PS-expressing tumor cells may survive the activation of the apoptosis cascade. Thus, there remains an unmet medical need for ensuring the complete cell death of PS-expression tumor cells.

### BRIEF DESCRIPTION OF THE FIGURES

FIG. 1A-FIG. 1C show the survival of phosphatidylserine-expressing AML cells. FIG. 1A shows FACS sorting of untreated c1498 cells (in DMEM with 8% FBS). The rectangles indicate the sorted cells. FIG. 1B shows FACS sorting of c1498 cells treated with cytarabine (1 μM) for 24 hrs). FIG. 1C shows the estimated live cell numbers in the sorted cells. About 1000 each of the sorted cells were placed into multi-well plates. When the cells reached sufficient numbers, they were counted at different time points to measure their growth rates. The growth rates were then used to estimate the initial live cell numbers. The following formula was used to calculate the minimal initial live cell numbers: Minimal initial cell number=Total cell population/(growth rate*t) (days to reach the current cell number).

FIG. 2A-FIG. 2D show the activities of PS-targeted CAR-T cells against various AML leukemia cells. Annexin V-CAR transduced T cells were mixed with AML leukemia cells (MVR-11, HL60, KG-la, and U937) labeled with the luciferase at different target (leukemia cells):effector (CAR-transduced T cells) ratios and media with 10% FBS for about 24 hours. The survival of the tumor cells was then measured by use of a plate reader. The values shown were normalized against tumor cells not incubated with the T cells. Y axis represents the relative luminescence values while the x-axis represents tumor cell vs CAR-T cell ratios.

FIG. 3 shows the anti-tumor efficacy of annexin V-CAR T cells against KG-la AML leukemia cells in vivo. About 5×10⁶ KG-la cells were injected subcutaneously into NSG mice on day 0. At 6 days post-inoculation, about 1×10⁷ control or Annexin V-CAR transduced T cells were injected into the tail vein of tumor-bearing mice (n=5 for each group). Tumor sizes were then measured until they reach 1.5 cm in diameter.

FIG. 4 shows possible structures of PS-targeting CARs as disclosed herein.

### BRIEF SUMMARY

Disclosed herein is a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein s a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor-specific antigen or fragment thereof.

Disclosed herein is an isolated nucleic acid molecule comprising a nucleic acid sequence encoding any disclosed CAR. Disclosed herein is an isolated nucleic acid molecule comprising a nucleic acid sequence encoding a CAR specific for phosphatidylserine (PS). Disclosed herein is an isolated nucleic acid molecule comprising a nucleic acid sequence encoding a CAR comprising an extracellular domain comprising an antigen binding domain, a transmembrane domain, and an intracellular domain comprising one or more immunostimulatory domains.

Disclosed herein a vector comprising an isolated nucleic acid molecule comprising a nucleic acid sequence encoding a CAR comprising an extracellular domain comprising an antigen binding domain, a transmembrane domain, and an intracellular domain comprising one or more immunostimulatory domains. Disclosed herein a vector comprising an isolated nucleic acid and an intracellular domain comprising one or more immunoreceptor tyrosine-based activation domains (ITAMs) and/or one or more co-stimulatory domains.

Disclosed herein are cells transformed or transfected by an isolated nucleic acid molecule comprising a nucleic acid sequence encoding a CAR specific for phosphatidylserine (PS).

Disclosed herein are cells transduced by a vector comprising an isolated nucleic acid molecule comprising a nucleic acid sequence encoding a CAR comprising an extracellular domain comprising an antigen binding domain, a transmembrane domain, and an intracellular domain comprising one or more immunostimulatory domains. Disclosed herein are cells transduced by a vector comprising an isolated nucleic acid molecule comprising a nucleic acid sequence encoding a CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains.

Disclosed herein are cells transduced by a vector comprising an isolated nucleic acid molecule comprising a nucleic acid sequence encoding a CAR comprising from N-terminus to C-terminus a signal peptide; an antigen binding domain; a hinge domain; a transmembrane domain; an intracellular domain, a cleavage signal; a signal peptide; and a tumor-specific antigen or fragment thereof. Disclosed herein are cells transduced by a vector comprising an isolated nucleic acid molecule comprising a nucleic acid sequence encoding a phosphatidylserine-specific CAR comprising an extracellular domain comprising an antigen binding domain, a transmembrane domain, and an intracellular domain comprising one or more immunostimulatory domains.

Disclosed herein is a method of treating cancer, the method comprising treating a subject in need thereof by administering to the subject in need thereof a therapeutically effective amount of one or more cells transduced with a disclosed recombinant vector or a pharmaceutical formulation thereof, wherein the one or more cells express a CAR.

Disclosed herein is a method of treating cancer, the method comprising treating a subject in need thereof by administering to the subject in need thereof a therapeutically effective amount of one or more cells transformed with a disclosed plasmid or a pharmaceutical formulation thereof, wherein the one or more cells express a CAR.

Disclosed herein is a method of treating cancer, the method comprising treating a subject in need thereof by administering to the subject in need thereof a therapeutically effective amount of one or more genetically modified cells expressing a chimeric antigen receptor targeting phosphatidylserine (PS) or a pharmaceutical formulation thereof.

Disclosed herein is a method of stimulating an effector cell-mediated immune modulator response to PS-expressing tumor cells, the method comprising administering to a subject in need thereof a therapeutically effective amount of one or more genetically modified cells expressing a chimeric antigen receptor targeting phosphatidylserine (PS) or a pharmaceutical formulation thereof.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, “operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element. In embodiments, a promotor is operably linked to nucleic acids.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have cancer, be suspected of having cancer, or be at risk of developing cancer.

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof, or by one or more of the disclosed methods. For example, “diagnosed with a disease or disorder” means having been subjected to an examination by a person of skill, for example, a physician, and found to have a condition (such as cancer) that can be treated by one or more the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof, or by one or more of the disclosed methods. For example, “suspected of having a disease or disorder” can mean having been subjected to an examination by a person of skill, for example, a physician, and found to have a condition (such as cancer) that can likely be treated by one or more of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof, or by one or more of the disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject afflicted with a disease or disorder (e.g., cancer). In an aspect, a patient can refer to a subject that has been diagnosed with or is suspected of having a disease or disorder such as cancer. In an aspect, a patient can refer to a subject that has been diagnosed with or is suspected of having a disease or disorder and is seeking treatment or receiving treatment for a disease or disorder (such as cancer).

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., cancer) based upon an earlier diagnosis by a person of skill and thereafter subjected to treatment for the cancer. In an aspect, the identification can be performed by a person different from the person making the diagnosis. In an aspect, the administration can be performed by one who performed the diagnosis.

As used herein, “activated” and “activation” can refer to the state of a T cell that has been sufficiently stimulated to induce detectable cellular proliferation. Activation can also be associated with induced cytokine production and detectable effector functions. The term “activated T cells” can refer to T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the TCR/CD3 complex.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not receiving the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not receiving the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof). In an aspect, the inhibition or reduction can be 0-25%, 25-50%, 50-75%, or 75-100% as compared to native or control levels. In an aspect, a native or control level can be a pre-disease or pre-disorder level (such as a pre-cancer state).

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having cancer). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as cancer). For example, treating a disease or disorder can reduce one or more symptoms of a disease or disorder in a subject by 1%-100% as compared to a control (such as, for example, an individual not having cancer). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder. It is understood that treatment does not necessarily refer to a cure or complete ablation or eradication of a disease or disorder. However, in an aspect, treatment can refer to a cure or complete ablation or eradication of a disease or disorder (such as cancer).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as cancer) or related complication from progressing to that complication. In an aspect, preventing metastasis is intended.

As used herein, the terms “administering” and “administration” refer to any method of providing one or more of the disclosed interfering molecules, the disclosed anti-PD1 molecules, the disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intratumoral administeraiton, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed composition, a disclosed pharmaceutical composition, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof can comprise administration directly into the CNS or the PNS. Administration can be continuous or intermittent. Administration can comprise a combination of one or more routes.

In an aspect, the skilled person can determine an efficacious dose, an efficacious schedule, and an efficacious route of administration for the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof to treat or prevent a disease or disorder (such as cancer). In an aspect, the skilled person can also alter, change, or modify an aspect of an administering step to improve efficacy of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof.

By “determining the amount” is meant both an absolute quantification of a particular analyte (e.g., biomarker for cancer, for example) or a determination of the relative abundance of a particular analyte (e.g., a cancer biomarker). The phrase includes both direct or indirect measurements of abundance or both.

As used herein, “modifying the method” can comprise modifying or changing one or more features or aspects of one or more steps of a disclosed method. In an aspect, a method can be altered by changing the amount of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof administered to a subject, or by changing the frequency of administration of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof to a subject, by changing the duration of time that the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof is administered to a subject, or by substituting for one or more of the disclosed components and/or reagents with a similar or equivalent component and/or reagent. The same applies to all disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or combinations thereof.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington″s Pharmaceutical Sciences, (1990) Mack Publishing Co., Easton, Pa., which is hereby incorporated by reference in its entirety.

A “transposon” is a mobile genetic element that efficiently moves between vectors and chromosomes using the “cut and paste” or “copy and paste” mechanism. During transposase transposition (for example, PB transposase in the PiggyBac transposon system) recognizes transposon-specific sequences of inverted terminal repeats (ITRs) located at both ends of the transposon (there are 5′- and 3′-ITRs in any transposon system), it moves the contents from source sites and embeds them in chromosomal sites, such as TTAA chromosomal sites. In an aspect, the powerful activity of the PiggyBac transposon system makes it easy to transfer genes of interest located between two ITRs to target genomes. The transposon can be divided into Class I transposon (retrotransposon) and Class II transposon (DNA transposon). In Class I transposon, after RNA is transcribed from nucleic acid in a cell or from transposon DNA on the animal genome, the DNA reverse-transcribed from the RNA is transferred to another location on the animal genome. It works by inserting it. Class II transposon cuts nucleic acid in cells or transposon DNA on the animal genome, and then inserts the cut transposon DNA into another location on the animal genome. The Class II transposon may include a first polynucleotide at a 5′ end, a second polynucleotide at a 3′ end, and a third polynucleotide. The first polynucleotide and the second polynucleotide may include an inverted terminal repeat (ITR) sequence. The third polynucleotide may be located between the first polynucleotide and the second polynucleotide. The third polynucleotide may include an exo-polynucleotide. The third polynucleotide may include a polynucleotide encoding a transposase. Unless otherwise specified below, the term “transposon” assumes the case of Class II transposon, but even if the term “transposon” is interpreted as Class I transposon, it is technically If there is no problem, it will not be necessary to limit the interpretation to Class II transposon.

As used herein, “concurrently” means (1) simultaneously in time, or (2) at different times during the course of a common treatment schedule.

The term “contacting” as used herein refers to bringing one or more of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof together with a target area or intended target area in such a manner that the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof can exert an effect on the intended target or targeted area either directly or indirectly. A target area or intended target area can be one or more of a subject's organs (e.g., lungs, heart, liver, kidney, brain, etc.) hosting cancerous cells. In an aspect, a target area or intended target area can be any cell or any organ infected by a disease or disorder (such as cancer). In an aspect, a target area or intended target area can be any organ, tissue, or cells that are affected by a disease or disorder (such as cancer).

As used herein, “determining” can refer to measuring or ascertaining the presence and severity of a disease or disorder, such as, for example, cancer. Methods and techniques used to determine the presence and/or severity of a disease or disorder are typically known to the medical arts. For example, the art is familiar with the ways to identify and/or diagnose the presence, severity, or both of a disease or disorder (such as, for example, cancer).

As used herein, “effective amount” and “amount effective” can refer to an amount that is sufficient to achieve the desired result such as, for example, the treatment and/or prevention of a disease or disorder (e.g., a cancer) or a suspected disease or disorder. As used herein, the terms “effective amount” and “amount effective” can refer to an amount that is sufficient to achieve the desired an effect on an undesired condition (e.g., a cancer). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed vector, a disclosed cell, or a disclosed pharmaceutical formulation; that (i) treats the particular disease, condition, or disorder (e.g., a cancer), (ii) attenuates, ameliorates, or eliminates one or more symptoms of the particular disease, condition, or disorder e.g., cancer), or (iii) delays the onset of one or more symptoms of the particular disease, condition, or disorder described herein (e.g., cancer). The specific therapeutically effective dose level for any particular patient will depend upon a variety of factors including the disorder being treated and the severity of the disorder; the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof employed; the disclosed methods employed; the age, body weight, general health, sex and diet of the patient; the time of administration; the route of administration; the rate of excretion of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof employed; the duration of the treatment; drugs used in combination or coincidental with the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof employed, and other like factors well known in the medical arts. For example, it is well within the skill of the art to start doses of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

The term “antibody” (Ab) includes, without limitation, a glycoprotein immunoglobulin that binds specifically to an antigen. An antibody can comprise at least two heavy (H) chains and two light (L) chains interconnected by disulfide bonds, or an antigen-binding molecule thereof. Each H chain can comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions can be further subdivided into regions of hypervariability, termed complementarity determining regions (CDRs), interspersed with regions that are more conserved, termed framework regions (FR). Each VH and VL can comprise three CDRs and four FRs, arranged from amino-terminus to carboxy-terminus in the following order: FR1, CDR1, FR2, CDR2, FR3, CDR3, and FR4. The variable regions of the heavy and light chains can contain a binding domain that interacts with an antigen. The constant regions of the Abs can mediate the binding of the immunoglobulin to host tissues or factors, including various cells of the immune system (e.g., effector cells) and the first component (Clq) of the classical complement system. Generally, human antibodies can be approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains can be linked or connected to one another by a single disulfide bond and two other disulfide bonds can connect the heavy chain hinge regions to one another, so that the dimers can be connected to one another and the tetramer can be formed. Naturally produced antibodies are also glycosylated, e.g., on the CH2 domain. The term “antibody” is used to mean an immunoglobulin molecule that recognizes and specifically binds to a target, such as a protein, polypeptide, peptide, carbohydrate, polynucleotide, lipid, or combinations of the foregoing etc., through at least one antigen recognition site within the variable region of the immunoglobulin molecule. As used herein, the term encompasses intact polyclonal antibodies, intact monoclonal antibodies, antibody fragments (such as Fab, Fab′, F (ab′) 2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD. IgE, IgG, and IgM, or subclasses (isotypes) thereof (e.g., IgG1. IgG2, IgG3. IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to—other molecules such as toxins, radioisotopes, etc.

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In embodiments, the variable region is a primate (e.g., non-human primate) variable region. In certain embodiments, the variable region comprises rodent or murine CDRs and primate framework regions (FRs).

The terms “VL” and “VL domain” are used interchangeably to refer to the light chain variable region of an antibody or an antigen-binding molecule thereof. The terms “VH” and “VH domain” are used interchangeably to refer to the heavy chain variable region of an antibody or an antigen-binding molecule thereof.

The terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

The term “heavy chain” when used in reference to an antibody can refer to any distinct type, e.g., alpha (a), delta (6), epsilon (E), gamma (y) and mu (p), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

The term “light chain” when used in reference to an antibody can refer to any distinct type, e.g., kappa (K) or lambda (A) based on the amino acid sequence of the constant domains. Light chain amino acid sequences are well known in the art. In specific embodiments, the light chain is a human light chain.

“Endogenous” with reference to a gene, protein, and/or nucleic acid refers to the natural presence of that gene, protein, and/or nucleic acid in a cell, such as an immune cell.

“Exogenous” refers to an introduced agent, such as a nucleic acid, gene, or protein, into a cell, for example from an outside source. A nucleic acid introduced into a cell is exogenous even if it encodes a protein which is naturally found in the cell. Such exogenous introduction of a nucleic acid encoding a protein can be used to increase the expression of the protein over the level that would naturally be found in the cell under similar conditions, e.g., without introduction of the exogenous nucleic acid.

A “T cell receptor” or “TCR” refers to antigen-recognition molecules present on the surface of T cells. During normal T cell development, each of the four TCR genes, a, 0., y, and 6, may rearrange leading to highly diverse TCR proteins.

As used herein, “effector function” can refer to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement mediated cytotoxicity (CMC). An effector function may be antigen binding dependent, antigen binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy can include, but are not limited to, NK cells and T cell therapies. T cell therapy can include adoptive T cell therapy, tumor-infiltrating lymphocyte (TIL) immunotherapy, autologous cell therapy, engineered autologous cell therapy (eACTTM), and allogeneic T cell transplantation. However, one of skill in the art would recognize that the conditioning methods disclosed herein would enhance the effectiveness of any transplanted T cell therapy.

The T cells or NK cells of the immunotherapy can come from any source known in the art. For example, T cells and NK cells can be differentiated in vitro from a hematopoietic stem cell population (for example iPSCs) or can be obtained from a subject. T cells and NK cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan.

As used herein, the term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multi-specific antibodies formed from antibody fragments.

A “monoclonal antibody” as used herein refers to homogenous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including, but not limited to, by hybridoma, phage selection, recombinant expression, and transgenic animals.

As used herein, the term “humanized antibody” refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues from the complementary determining region (CDR) are replaced by residues from the CDR of a non-human species (e.g., mouse, rat, rabbit, hamster, etc.) that have the desired specificity, affinity, and capability. In some instances, the Fv framework region (FR) residues of a human immunoglobulin are replaced with the corresponding residues in an antibody from a non-human species that has the desired specificity, affinity, and capability. The humanized antibody can be further modified by the substitution of additional residue either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin.

That an antibody “selectively binds” or “specifically binds” to an epitope or receptor means that the antibody reacts or associates more frequently, more rapidly, with greater duration, with greater affinity, or with some combination of the above to the epitope or receptor than with alternative substances, including unrelated proteins. “Selectively binds” or “specifically binds” means, for instance, that an antibody binds to a protein with a K_{D} of about 0.1 mM or less, more usually about 1 μM or less. “Selectively binds” or “specifically binds” means at times that an antibody binds to a protein with a K_{D} of about 0.1 mM or less, at times about 1 μM or less, at times about 0.1 μM or less, at times about 0.01 μM or less, and at times about 1 nM or less. It is understood that, in certain embodiments, an antibody or binding moiety that specifically binds to a first target may or may not specifically bind to a second target. As such, “specific binding” does not necessarily require (although it can include) exclusive binding, e.g., binding to a single target. Thus, an antibody may, in an aspect, specifically bind to more than one target (e.g., human phosphatidylserine or PS). In an aspect, the multiple targets may be bound by the same antigen-binding site on the antibody. For example, an antibody may, in certain instances, comprise two identical antigen-binding sites, each of which specifically binds two or more human Notch receptors (e.g., human PS).

An “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind.

A “target” is any molecule bound by a binding motif, CAR, TCR or antigen binding agent, e.g., an antibody.

“Antigen-specific targeting region” (ASTR) refers to the region of the CAR or TCR which targets specific antigens. The targeting regions on the CAR or TCR are extracellular (for example, phosphatidylserine). In some embodiments, the antigen-specific targeting regions comprise an antibody or a functional equivalent thereof or a fragment thereof or a derivative thereof and each of the targeting regions target a different antigen. The targeting regions may comprise full length heavy chain, Fab fragments, single chain Fv (scFv) fragments, divalent single chain antibodies or diabodies, each of which are specific to the target antigen.

The term “autologous” refers to any material derived from the same individual to which it is later to be re-introduced. For example, a subject's own cells can be obtained, made to express one or more disclosed CARs, and then administered to the same subject.

As used herein, the term “antibody production” can have both general and specific meanings. In the broad sense, it can refer to the entire process of creating a usable specific antibody, including steps of immunogen preparation, immunization, hybridoma creation, collection, screening, isotyping, purification, and labeling for direct use in a particular method. In the more restricted sense, antibody production refers to the steps leading up to antibody generation but does not include various forms of purifying and labeling the antibody for particular uses. Antibody production involves preparation of antigen samples and their safe injection into laboratory or farm animals to evoke high expression levels of antigen-specific antibodies in the serum, which can then be recovered from the animal. Polyclonal antibodies are recovered directly from serum (bleeds). Monoclonal antibodies are produced by fusing antibody-secreting spleen cells from immunized mice with immortal myeloma cell to create monoclonal hybridoma cell lines that express the specific antibody in cell culture supernatant. Successful antibody production depends upon careful planning and implementation with respect to several important steps and considerations: (i) synthesize or purify the target antigen (e.g., peptide or hapten); (ii) choose an appropriate immunogenic carrier protein; (iii) conjugate the antigen and carrier protein to create the immunogen; immunize animals using appropriate schedule and adjuvant formula; and screen serum (or hybridoma) for antibody titer and isotype (also called antibody characterization).

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The terms “proliferative disorder” and “proliferative disease” refer to disorders associated with abnormal cell proliferation such as cancer.

“Tumor” and “neoplasm” as used herein refer to any mass of tissue that result from excessive cell growth or proliferation, either benign (noncancerous) or malignant (cancerous) including pre-cancerous lesions. “Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” or “tumor stem cell” or “solid tumor stem cell” are used interchangeably herein and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of “cancer stem cells” or “tumor stem cells” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell” and grammatical equivalents refer to the total population of cells derived from a tumor including both non-tumorigenic cells, which comprise the bulk of the tumor cell population, and tumorigenic stem cells (cancer stem cells).

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor.

As used herein, the “tumorigenicity” of a tumor refers to the ability of a random sample of cells from the tumor to form palpable tumors upon serial transplantation into immunocompromised mice.

As used herein, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. In an aspect, the term cationic and ionizable in the context of LNP lipids can be use interchangeably, e.g., wherein ionizable lipids are cationic depending on the pH.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGFIR antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., the disclosed isolated nucleic acid molecules, disclosed vectors, disclosed cells (e.g., disclosed genetically modified cells expressing a chimeric antigen receptor targeting phosphatidylserine (PS) or the disclosed engineered T cells or NK cells or macrophages expressing a chimeric antigen receptor targeting phosphatidylserine (PS)), disclosed pharmaceutical formulations, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy to a subject having or diagnosed with cancer.

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Chimeric Antigen Receptors

“Chimeric antigen receptor” or “CAR” refers to a molecule engineered to comprise a binding domain and a means of activating immune cells (for example T cells such as naive T cells, central memory T cells, effector memory T cells, NK cells or combination thereof) upon antigen binding. CARs are also known as artificial T cell receptors, chimeric T cell receptors or chimeric immunoreceptors. In some embodiments, a CAR comprises a binding domain, an extracellular domain, a transmembrane domain, one or more co-stimulatory domains, and an intracellular signaling domain. A T cell that has been genetically engineered to express a chimeric antigen receptor may be referred to as a CAR T cell. Similarly, an NK cell that has been genetically engineered to express a chimeric antigen receptor may be referred to as a CAR NK cell.

Chimeric antigen receptors (CARs) are proteins which graft the specificity of a monoclonal antibody (mAb) to the effector function of a T-cell. CARs comprise an extracellular ligand-binding domain, most commonly a single chain variable fragment (scFv), a spacer domain, a transmembrane domain, and one or more cytoplasmic domains.

First-generation CARs contain a single activatory domain (e.g., the CD3ζ cytoplasmic domain). Second-generation CARs comprise an activatory domain (e.g., CD3ζ or γ chain of Fc receptors) connected to co-stimulatory domains obtained from native co-stimulatory molecules such as CD28 and 4-1BB. Third-generation CARs incorporate CD3ζ with two co-stimulatory cytoplasmic domains. The design of each module of the CAR structure can contribute to CAR-T-cell signaling mechanisms, effector functions, and its eventual efficacy and toxicity. It is evident that modules such as the scFv and intracellular cytoplasmic domains play a key role in ligand recognition and signaling. It has recently become clear that non-signaling domains such as the spacer and transmembrane domains can also influence CAR functions.

scFvs are the most commonly used ligand-binding domains in CAR structures. scFv affinity is a key parameter that has been modulated to improve specificity of the CAR and reduce “on-target, off-tumor” side effects, which is of particular importance when the target antigen is ubiquitously expressed on healthy tissue.

Spacer domains that connect the scFv to the transmembrane domain lend flexibility to the scFv and help improve efficacy. Appropriate spacer domain engineering can enable recognition of target epitopes that are otherwise sterically inaccessible. Spacer domain modulation can also be used to regulate synaptic cleft distances and hence signaling phenomena such as kinetic segregation. To maintain optimal synapse distance, membrane-distal epitopes usually require shorter spacers whereas membrane-proximal epitopes require longer spacers. Apart from limiting exclusion of inhibitory phosphatases, increasing epitope-paratope distance can also result in impaired delivery of granzymes and perforins to the target cell, thus reducing lytic efficiency. In a physiological T-cell setting, the highly dense immune synapse hinders diffusion of lytic granules, which enhances pore formation by perforins and granzyme delivery [

Transmembrane domains in CAR structures serve as a fulcrum for transducing ligand recognition signals to the intracellular cytoplasmic domain.

In T cells, the intracellular signaling domain of the TCR-CD3 complex transduces the necessary “signal 1” to kick-start the signaling cascade. Co-stimulatory receptors, especially CD28, convey “signal 2” which is important for sustained signaling, prevention of anergy, and proliferation. 4-1BB, ICOS, and OX40 are other co-stimulatory receptors that affect T-cell differentiation pathways, metabolic cycles, as well as apoptosis and activation-induced cell death.

In CAR-T cells, co-stimulatory signals are usually included in-cis with the CD35 cytoplasmic domain. The number and type of co-stimulatory signals, as well as their order and proximity to the membrane, are critical parameters that have been addressed in literature (FIG. 5). Immunoreceptor Tyrosine Activation Motifs (ITAMs) present on cytoplasmic domains of TCR-CD3 complexes are the phosphorylation sites, which recruit ZAP70, critical for signaling cascades. In T cells, ITAM diversity and number of functional CD32 ITAMs are important for optimal signaling. In CAR-T cells, the number of functional ITAMs is gaining attention as an important design strategy to ensure efficacy.

### C. Annexins

ANXA1 (Annexin A1) is a protein coding gene. This gene encodes a membrane-localized protein that binds phospholipids. This protein inhibits phospholipase A2 and has anti-inflammatory activity. Loss of function or expression of this gene has been detected in multiple tumors. Diseases associated with ANXA1 include Shoulder Impingement Syndrome and Brain Edema. Among its related pathways are GPCR downstream signaling and Class A/1 (Rhodopsin-like receptors). Gene Ontology (GO) annotations related to this gene include calcium ion binding and signaling receptor binding. An important paralog of this gene is ANXA2. ANXA1 plays important roles in the innate immune response as effector of glucocorticoid-mediated responses and regulator of the inflammatory process. ANXA1 has anti-inflammatory activity. ANXA1 plays a role in glucocorticoid-mediated down-regulation of the early phase of the inflammatory response (by similarity). ANXA1 contributes to the adaptive immune response by enhancing signaling cascades that are triggered by T-cell activation, regulates differentiation and proliferation of activated T-cells. ANXA1 promotes the differentiation of T-cells into Th1 cells and negatively regulates differentiation into Th2 cells. ANXA1 has no effect on unstimulated T cells. ANXA1 negatively regulates hormone exocytosis via activation of the formyl peptide receptors and reorganization of the actin cytoskeleton. ANXA1 has high affinity for Ca (2+) and can bind up to eight Ca (2+) ions (by similarity). ANXA1 displays Ca (2+)-dependent binding to phospholipid membranes. ANXA1 plays a role in the formation of phagocytic cups and phagosomes and plays a role in phagocytosis by mediating the Ca (2+)-dependent interaction between phagosomes and the actin cytoskeleton (by similarity). ANXA1 functions at least in part by activating the formyl peptide receptors and downstream signaling cascades. ANXA1 promotes chemotaxis of granulocytes and monocytes via activation of the formyl peptide receptors and promotes rearrangement of the actin cytoskeleton, cell polarization and cell migration. ANXA1 promotes resolution of inflammation and wound healing. ANXA1 acts via neutrophil N-formyl peptide receptors to enhance the release of CXCL2.

ANXA2 (Annexin A2) is a protein coding gene. This gene encodes a member of the annexin family. Members of this calcium-dependent phospholipid-binding protein family play a role in the regulation of cellular growth and in signal transduction pathways. This protein functions as an autocrine factor which heightens osteoclast formation and bone resorption. This gene has three pseudogenes located on chromosomes 4, 9 and 10, respectively. Multiple alternatively spliced transcript variants encoding different isoforms have been found for this gene. Annexin A2 expression has been found to correlate with resistance to treatment against various cancer forms. Diseases associated with ANXA2 include Antiphospholipid Syndrome and Acute Promyelocytic Leukemia. Among its related pathways are Interleukin-12 family signaling and Innate Immune System. Gene Ontology (GO) annotations related to this gene include RNA binding and small GTPase binding. An important paralog of this gene is ANXA1. Calcium-regulated membrane-binding protein whose affinity for calcium is greatly enhanced by anionic phospholipids. It binds two calcium ions with high affinity. May be involved in heat-stress response. Inhibits PCSK9-enhanced LDLR degradation, probably reduces PCSK9 protein levels via a translational mechanism but also competes with LDLR for binding with PCSK9. ANXA2 binds M. pneumoniae CARDS toxin, probably serves as one receptor for this pathogen. When ANXA2 is down-regulated by siRNA, less toxin binds to human cells and less vacuolization (a symptom of M. pneumoniae infection) is seen.

ANXA3 (Annexin A3) is a protein coding gene. This gene encodes a member of the annexin family. Members of this calcium-dependent phospholipid-binding protein family play a role in the regulation of cellular growth and in signal transduction pathways. This protein functions in the inhibition of phospholipase A2 and cleavage of inositol 1,2-cyclic phosphate to form inositol 1-phosphate. This protein may also play a role in anti-coagulation. Diseases associated with ANXA3 include ovarian cancer and prostate cancer. Among its related pathways are prostaglandin synthesis and regulation. Gene Ontology (GO) annotations related to this gene include calcium ion binding and calcium-dependent phospholipid binding. An important paralog of this gene is ANXA11. Inhibitor of phospholipase A2, also possesses anti-coagulant properties. Also cleaves the cyclic bond of inositol 1,2-cyclic phosphate to form inositol 1-phosphate.

ANXA4 (Annexin A4) is a protein coding gene. Annexin IV (ANX4) belongs to the annexin family of calcium-dependent phospholipid binding proteins. Although their functions are still not clearly defined, several members of the annexin family have been implicated in membrane-related events along exocytotic and endocytotic pathways. ANX4 has 45 to 59% identity with other members of its family and shares a similar size and exon-intron organization. Isolated from human placenta, ANX4 encodes a protein that has possible interactions with ATP and has in vitro anticoagulant activity and also inhibits phospholipase A2 activity. ANX4 is almost exclusively expressed in epithelial cells. Several transcript variants encoding different isoforms have been found for this gene. Among its related pathways are prostaglandin synthesis and regulation. Gene Ontology (GO) annotations related to this gene include calcium ion binding and calcium-dependent protein binding. An important paralog of this gene is ANXA11. Calcium/phospholipid-binding protein which promotes membrane fusion and is involved in exocytosis.

ANXA5 (Annexin A5) is a protein coding gene. The Annexin 5 gene spans 29 kb containing 13 exons, and encodes a single transcript of approximately 1.6 kb and a protein product with a molecular weight of about 35 kDa. The protein encoded by this gene belongs to the annexin family of calcium-dependent phospholipid binding proteins some of which have been implicated in membrane-related events along exocytotic and endocytotic pathways. Annexin 5 is a phospholipase A2 and protein kinase C inhibitory protein with calcium channel activity and a potential role in cellular signal transduction, inflammation, growth and differentiation. Annexin 5 has also been described as placental anticoagulant protein I, vascular anticoagulant-alpha, endonexin II, lipocortin V, placental protein 4 and anchorin CII. Polymorphisms in this gene have been implicated in various obstetric complications. Diseases associated with ANXA5 include Pregnancy Loss, Recurrent 3 and Antiphospholipid Syndrome. Among its related pathways are Response to elevated platelet cytosolic Ca2+ and Regulation of CFTR activity (norm and CF). Gene Ontology (GO) annotations related to this gene include calcium ion binding and calcium-dependent phospholipid binding. An important paralog of this gene is ANXA4. This protein is an anticoagulant protein that acts as an indirect inhibitor of the thromboplastin-specific complex, which is involved in the blood coagulation cascade.

ANXA6 (Annexin A6) is a protein coding gene. Annexin VI belongs to a family of calcium-dependent membrane and phospholipid binding proteins. Several members of the annexin family have been implicated in membrane-related events along exocytotic and endocytotic pathways. The annexin VI gene is approximately 60 kbp long and contains 26 exons. It encodes a protein of about 68 kDa that consists of eight 68-amino acid repeats separated by linking sequences of variable lengths. It is highly similar to human annexins I and II sequences, each of which contain four such repeats. Annexin VI has been implicated in mediating the endosome aggregation and vesicle fusion in secreting epithelia during exocytosis. Alternatively spliced transcript variants have been described. Diseases associated with ANXA6 include Kwashiorkor and Malignant Hyperthermia. Among its related pathways are Cardiac conduction and Myometrial relaxation and contraction pathways. Gene Ontology (GO) annotations related to this gene include calcium ion binding and GTP binding. An important paralog of this gene is ANXA11.

ANXA7 (Annexin A7) is a protein coding gene. Annexin VII is a member of the annexin family of calcium-dependent phospholipid binding proteins. The Annexin VII gene contains 14 exons and spans approximately 34 kb of DNA. An alternatively spliced cassette exon results in two mRNA transcripts of 2.0 and 2.4 kb which are predicted to generate two protein isoforms differing in their N-terminal domain. The alternative splicing event is tissue specific and the mRNA containing the cassette exon is prevalent in brain, heart and skeletal muscle. The transcripts also differ in their 3″-non coding regions by the use of two alternative poly(A) signals. Annexin VII encodes a protein with a molecular weight of approximately 51 kDa with a unique, highly hydrophobic N-terminal domain of 167 amino acids and a conserved C-terminal region of 299 amino acids. The latter domain is composed of alternating hydrophobic and hydrophilic segments. Structural analysis of the protein suggests that Annexin VII is a membrane binding protein with diverse properties, including voltage-sensitive calcium channel activity, ion selectivity and membrane fusion. Among its related pathways are Cytoskeletal Signaling and Ca, CAMP and Lipid Signaling. Gene Ontology (GO) annotations related to this gene include RNA binding and integrin binding. An important paralog of this gene is ANXA11.

ANXA8 (Annexin A8) is a protein coding gene. This gene encodes a member of the annexin family of evolutionarily conserved Ca2+ and phospholipid binding proteins. The encoded protein may function as an anticoagulant that indirectly inhibits the thromboplastin-specific complex. Overexpression of this gene has been associated with acute myelocytic leukemia. A highly similar duplicated copy of this gene is found in close proximity on the long arm of chromosome 10. Diseases associated with ANXA8 include Breast Adenocarcinoma and Heterophyiasis. Gene Ontology (GO) annotations related to this gene include calcium ion binding and calcium-dependent phospholipid binding. An important paralog of this gene is ANXA8L1. This protein is an anticoagulant protein that acts as an indirect inhibitor of the thromboplastin-specific complex, which is involved in the blood coagulation cascade.

ANXA8L1 (Annexin A8 Like 1) is a protein coding gene. Among its related pathways are Prostaglandin synthesis and regulation. Gene Ontology (GO) annotations related to this gene include calcium ion binding and calcium-dependent phospholipid binding. An important paralog of this gene is ANXA8. This gene encodes a member of the annexin family of evolutionarily conserved Ca2+ and phospholipid binding proteins. The encoded protein may function as an anticoagulant that indirectly inhibits the thromboplastin-specific complex. Overexpression of this gene has been associated with acute myelocytic leukemia. A highly similar duplicated copy of this gene is found in close proximity on the long arm of chromosome 10.

ANXA9 (Annexin A9) is a protein coding gene. The annexins are a family of calcium-dependent phospholipid-binding proteins. Members of the annexin family contain 4 internal repeat domains, each of which includes a type II calcium-binding site. The calcium-binding sites are required for annexins to aggregate and cooperatively bind anionic phospholipids and extracellular matrix proteins. This gene encodes a divergent member of the annexin protein family in which all four homologous type II calcium-binding sites in the conserved tetrad core contain amino acid substitutions that ablate their function. However, structural analysis suggests that the conserved putative ion channel formed by the tetrad core is intact. Diseases associated with ANXA9 include Pemphigus and Acantholytic Acanthoma. Gene Ontology (GO) annotations related to this gene include calcium ion binding and phospholipid binding. An important paralog of this gene is ANXA2. Low affinity receptor for acetylcholine known to be targeted by disease-causing pemphigus vulgaris antibodies in keratinocytes.

ANXA10 (Annexin A10) is a protein coding gene. This gene encodes a member of the annexin family. Members of this calcium-dependent phospholipid-binding protein family play a role in the regulation of cellular growth and in signal transduction pathways. The function of this gene has not yet been determined. Diseases associated with ANXA10 include Hepatocellular Carcinoma. Gene Ontology (GO) annotations related to this gene include calcium ion binding and calcium-dependent phospholipid binding. An important paralog of this gene is ANXA4.

ANXA11 (Annexin A11) is a protein coding gene. This gene encodes a member of the annexin family, a group of calcium-dependent phospholipid-binding proteins. Annexins have unique N-terminal domains and conserved C-terminal domains, which contain calcium-dependent phospholipid-binding sites. The encoded protein is a 56-kD antigen recognized by sera from patients with various autoimmune diseases. Several transcript variants encoding two different isoforms have been identified. Diseases associated with ANXA11 include Amyotrophic Lateral Sclerosis 23 and Inclusion Body Myopathy 0nd Brain White Matter Abnormalities. Gene Ontology (GO) annotations related to this gene include RNA binding and calcium-dependent protein binding. An important paralog of this gene is ANXA7. Binds specifically to calcyclin in a calcium-dependent manner (By similarity). Required for midbody formation and completion of the terminal phase of cytokinesis.

ANXA13 (Annexin A13) is a protein coding gene. This gene encodes a member of the annexin family. Members of this calcium-dependent phospholipid-binding protein family play a role in the regulation of cellular growth and in signal transduction pathways. The specific function of this gene has not yet been determined; however, it is associated with the plasma membrane of undifferentiated, proliferating endothelial cells and differentiated villus enterocytes. Alternatively spliced transcript variants encoding different isoforms have been identified. Gene Ontology (GO) annotations related to this gene include calcium ion binding and phosphatidylserine binding. An important paralog of this gene is ANXA8.

### D. Compositions for Use in the Disclosed Methods

### 1. Chimeric Antigen Receptors

Disclosed herein are chimeric antigen receptors. Disclosed herein are chimeric antigen receptors for phosphatidylserine (PS). Disclosed herein is a chimeric antigen receptor (CAR), comprising an extracellular domain comprising an antigen binding domain, a transmembrane domain, and an intracellular domain comprising one or more immunostimulatory domains.

In an aspect, a disclosed the extracellular domain can further comprise a signal peptide. In an aspect, a disclosed signal peptide can comprise a CD8 signal peptide.

In an aspect, a disclosed CD8 signal peptide can comprise the sequence set forth in SEQ ID NO: 08 or a fragment thereof.

In an aspect, a disclosed CD8 signal peptide can comprise a sequence having at least 70%, at least 75%, at least 80%, at least 85%, at least 90%, or at least 95% identity to the sequence set forth in SEQ ID NO:08 or a fragment thereof.

In an aspect, a disclosed antigen binding domain can comprise a phosphatidylserine (PS) binding domain. In an aspect, a disclosed PS binding domain can comprise Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect, a disclosed ANXA1 can comprise the amino acid sequence set forth in SEQ ID NO: 19 or a fragment thereof. In an aspect, a disclosed ANXA1 signal peptide can comprise a sequence having at least 70%, at least 75%, at least 80%, at least 85%, at least 90%, or at least 95% identity to the sequence set forth in SEQ ID NO:19 or a fragment thereof. In an aspect, a disclosed ANXA1 can comprise the amino acid sequence set forth in SEQ ID NO:20 or a fragment thereof. In an aspect, a disclosed ANXA1 signal peptide can comprise a sequence having at least 70%, at least 75%, at least 80%, at least 85%, at least 90%, or at least 95% identity to the sequence set forth in SEQ ID NO:20 or a fragment thereof. In an aspect, a disclosed ANXA2 can comprise the amino acid sequence set forth in SEQ ID NO:21 or a fragment thereof. In an aspect, a disclosed ANXA2 signal peptide can comprise a sequence having at least 70%, at least 75%, at least 80%, at least 85%, at least 90%, or at least 95% identity to the sequence set forth in SEQ ID NO:21 or a fragment thereof. In an aspect, a disclosed ANXA3 can comprise the amino acid sequence set forth in SEQ ID NO:22 or a fragment thereof. In an aspect, a disclosed ANXA3 signal peptide can comprise a sequence having at least 70%, at least 75%, at least 80%, at least 85%, at least 90%, or at least 95% identity to the sequence set forth in SEQ ID NO:22 or a fragment thereof. In an aspect, a disclosed ANXA4 can comprise the amino acid sequence set forth in SEQ ID NO: 23 or a fragment thereof. In an aspect, a disclosed ANXA4 signal peptide can comprise a sequence having at least 70%, at least 75%, at least 80%, at least 85%, at least 90%, or at least 95% identity to the sequence set forth in SEQ ID NO:23 or a fragment thereof. In an aspect, a disclosed ANXA5 can comprise the amino acid sequence set forth in SEQ ID NO:24 or a fragment thereof. In an aspect, a disclosed ANXA5 signal peptide can comprise a sequence having at least 70%, at least 75%, at least 80%, at least 85%, at least 90%, or at least 95% identity to the sequence set forth in SEQ ID NO:24 or a fragment thereof. In an aspect, a disclosed ANXA6 can comprise the amino acid sequence set forth in SEQ ID NO:25 or a fragment thereof. In an aspect, a disclosed ANXA6 signal peptide can comprise a sequence having at least 70%, at least 75%, at least 80%, at least 85%, at least 90%, or at least 95% identity to the sequence set forth in SEQ ID NO:25 or a fragment thereof. In an aspect, a disclosed ANXA7 can comprise the amino acid sequence set forth in SEQ ID NO:26 or a fragment thereof. In an aspect, a disclosed ANXA7 signal peptide can comprise a sequence having at least 70%, at least 75%, at least 80%, at least 85%, at least 90%, or at least 95% identity to the sequence set forth in SEQ ID NO:26 or a fragment thereof. In an aspect, a disclosed ANXA8 can comprise the amino acid sequence set forth in SEQ ID NO: 27 or a fragment thereof. In an aspect, a disclosed ANXA8 signal peptide can comprise a sequence having at least 70%, at least 75%, at least 80%, at least 85%, at least 90%, or at least 95% identity to the sequence set forth in SEQ ID NO:27 or a fragment thereof. In an aspect, a disclosed ANXA8L1 can comprise the amino acid sequence set forth in SEQ ID NO:28 or a fragment thereof. In an aspect, a disclosed ANXA8L1 signal peptide can comprise a sequence having at least 70%, at least 75%, at least 80%, at least 85%, at least 90%, or at least 95% identity to the sequence set forth in SEQ ID NO:28 or a fragment thereof. In an aspect, a disclosed ANXA9 can comprise the amino acid sequence set forth in SEQ ID NO:29 or a fragment thereof. In an aspect, a disclosed ANXA9 signal peptide can comprise a sequence having at least 70%, at least 75%, at least 80%, at least 85%, at least 90%, or at least 95% identity to the sequence set forth in SEQ ID NO:29 or a fragment thereof. In an aspect, a disclosed ANXA10 can comprise the amino acid sequence set forth in SEQ ID NO:30 or a fragment thereof. In an aspect, a disclosed ANXA10 signal peptide can comprise a sequence having at least 70%, at least 75%, at least 80%, at least 85%, at least 90%, or at least 95% identity to the sequence set forth in SEQ ID NO:30 or a fragment thereof. In an aspect, a disclosed ANXA11 can comprise the amino acid sequence set forth in SEQ ID NO:31 or a fragment thereof. In an aspect, a disclosed ANXA11 signal peptide can comprise a sequence having at least 70%, at least 75%, at least 80%, at least 85%, at least 90%, or at least 95% identity to the sequence set forth in SEQ ID NO:31 or a fragment thereof. In an aspect, a disclosed ANXA13 can comprise the amino acid sequence set forth in SEQ ID NO:32 or a fragment thereof. In an aspect, a disclosed ANXA13 signal peptide can comprise a sequence having at least 70%, at least 75%, at least 80%, at least 85%, at least 90%, or at least 95% identity to the sequence set forth in SEQ ID NO:32 or a fragment thereof.

In an aspect, a disclosed ADGRB1 can comprise the amino acid sequence set forth in SEQ ID NO: 33 or a fragment thereof. In an aspect, a disclosed ADGRB1 signal peptide can comprise a sequence having at least 70%, at least 75%, at least 80%, at least 85%, at least 90%, or at least 95% identity to the sequence set forth in SEQ ID NO:33 or a fragment thereof. In an aspect, a disclosed ADGRB1 can comprise the amino acid sequence set forth in SEQ ID NO:34 or a fragment thereof. In an aspect, a disclosed ADGRB1 signal peptide can comprise a sequence having at least 70%, at least 75%, at least 80%, at least 85%, at least 90%, or at least 95% identity to the sequence set forth in SEQ ID NO:34 or a fragment thereof. In an aspect, a disclosed APO-H can comprise the amino acid sequence set forth in SEQ ID NO:35 or a fragment thereof. In an aspect, a disclosed APO-H can comprise a sequence having at least 70%, at least 75%, at least 80%, at least 85%, at least 90%, or at least 95% identity to the sequence set forth in SEQ ID NO:35 or a fragment thereof. In an aspect, a disclosed F2 can comprise the amino acid sequence set forth in SEQ ID NO:36 or a fragment thereof. In an aspect, a disclosed F2 can comprise a sequence having at least 70%, at least 75%, at least 80%, at least 85%, at least 90%, or at least 95% identity to the sequence set forth in SEQ ID NO:36 or a fragment thereof. In an aspect, a disclosed F7 can comprise the amino acid sequence set forth in SEQ ID NO:37 or a fragment thereof. In an aspect, a disclosed F7 can comprise a sequence having at least 70%, at least 75%, at least 80%, at least 85%, at least 90%, or at least 95% identity to the sequence set forth in SEQ ID NO:37 or a fragment thereof. In an aspect, a disclosed F9 can comprise the amino acid sequence set forth in SEQ ID NO: 38 or a fragment thereof. In an aspect, a disclosed F9 can comprise a sequence having at least 70%, at least 75%, at least 80%, at least 85%, at least 90%, or at least 95% identity to the sequence set forth in SEQ ID NO:38 or a fragment thereof. In an aspect, a disclosed F10 can comprise the amino acid sequence set forth in SEQ ID NO:39 or a fragment thereof. In an aspect, a disclosed F10 can comprise a sequence having at least 70%, at least 75%, at least 80%, at least 85%, at least 90%, or at least 95% identity to the sequence set forth in SEQ ID NO:39 or a fragment thereof. In an aspect, a disclosed GAS6 can comprise the amino acid sequence set forth in SEQ ID NO:40 or a fragment thereof. In an aspect, a disclosed GAS6 an comprise a sequence having at least 70%, at least 75%, at least 80%, at least 85%, at least 90%, or at least 95% identity to the sequence set forth in SEQ ID NO:40 or a fragment thereof. In an aspect, a disclosed MFGE8 can comprise the amino acid sequence set forth in SEQ ID NO:41 or a fragment thereof. In an aspect, a disclosed MFGE8 can comprise a sequence having at least 70%, at least 75%, at least 80%, at least 85%, at least 90%, or at least 95% identity to the sequence set forth in SEQ ID NO:41 or a fragment thereof. In an aspect, a disclosed AGER can comprise the amino acid sequence set forth in SEQ ID NO: 42 or a fragment thereof. In an aspect, a disclosed AGER can comprise a sequence having at least 70%, at least 75%, at least 80%, at least 85%, at least 90%, or at least 95% identity to the sequence set forth in SEQ ID NO:42 or a fragment thereof. In an aspect, a disclosed AGER can comprise the amino acid sequence set forth in SEQ ID NO:43 or a fragment thereof. In an aspect, a disclosed AGER can comprise a sequence having at least 70%, at least 75%, at least 80%, at least 85%, at least 90%, or at least 95% identity to the sequence set forth in SEQ ID NO:43 or a fragment thereof. In an aspect, a disclosed PROS1 can comprise the amino acid sequence set forth in SEQ ID NO:44 or a fragment thereof. In an aspect, a disclosed PROS1 can comprise a sequence having at least 70%, at least 75%, at least 80%, at least 85%, at least 90%, or at least 95% identity to the sequence set forth in SEQ ID NO:44 or a fragment thereof. In an aspect, a disclosed STAB1 can comprise the amino acid sequence set forth in SEQ ID NO:45 or a fragment thereof. In an aspect, a disclosed STAB1 can comprise a sequence having at least 70%, at least 75%, at least 80%, at least 85%, at least 90%, or at least 95% identity to the sequence set forth in SEQ ID NO:45 or a fragment thereof. In an aspect, a disclosed STAB1 can comprise the amino acid sequence set forth in SEQ ID NO:46 or a fragment thereof. In an aspect, a disclosed STAB1 can comprise a sequence having at least 70%, at least 75%, at least 80%, at least 85%, at least 90%, or at least 95% identity to the sequence set forth in SEQ ID NO:46 or a fragment thereof. In an aspect, a disclosed STAB2 can comprise the amino acid sequence set forth in SEQ ID NO:47 or a fragment thereof. In an aspect, a disclosed STAB2 can comprise a sequence having at least 70%, at least 75%, at least 80%, at least 85%, at least 90%, or at least 95% identity to the sequence set forth in SEQ ID NO:47 or a fragment thereof. In an aspect, a disclosed STAB2 can comprise the amino acid sequence set forth in SEQ ID NO:48 or a fragment thereof. In an aspect, a disclosed STAB2 can comprise a sequence having at least 70%, at least 75%, at least 80%, at least 85%, at least 90%, or at least 95% identity to the sequence set forth in SEQ ID NO:48 or a fragment thereof.

In an aspect, a disclosed HAVCR1 can comprise the amino acid sequence set forth in SEQ ID NO: 49 or a fragment thereof. In an aspect, a disclosed HAVCR1 can comprise a sequence having at least 70%, at least 75%, at least 80%, at least 85%, at least 90%, or at least 95% identity to the sequence set forth in SEQ ID NO:49 or a fragment thereof. In an aspect, a disclosed HAVCR1 can comprise the amino acid sequence set forth in SEQ ID NO:50 or a fragment thereof. In an aspect, a disclosed HAVCR1 can comprise a sequence having at least 70%, at least 75%, at least 80%, at least 85%, at least 90%, or at least 95% identity to the sequence set forth in SEQ ID NO: 50 or a fragment thereof. In an aspect, a disclosed HAVCR2 can comprise the amino acid sequence set forth in SEQ ID NO:51 or a fragment thereof. In an aspect, a disclosed HAVCR2 can comprise a sequence having at least 70%, at least 75%, at least 80%, at least 85%, at least 90%, or at least 95% identity to the sequence set forth in SEQ ID NO:51 or a fragment thereof. In an aspect, a disclosed HAVCR2 can comprise the amino acid sequence set forth in SEQ ID NO: 52 or a fragment thereof. In an aspect, a disclosed HAVCR2 can comprise a sequence having at least 70%, at least 75%, at least 80%, at least 85%, at least 90%, or at least 95% identity to the sequence set forth in SEQ ID NO:52 or a fragment thereof. In an aspect, a disclosed TIMD4 can comprise the amino acid sequence set forth in SEQ ID NO:53 or a fragment thereof. In an aspect, a disclosed TIMD4 can comprise a sequence having at least 70%, at least 75%, at least 80%, at least 85%, at least 90%, or at least 95% identity to the sequence set forth in SEQ ID NO:53 or a fragment thereof. In an aspect, a disclosed TIMD4 can comprise the amino acid sequence set forth in SEQ ID NO:54 or a fragment thereof. In an aspect, a disclosed TIMD4 can comprise a sequence having at least 70%, at least 75%, at least 80%, at least 85%, at least 90%, or at least 95% identity to the sequence set forth in SEQ ID NO:54 or a fragment thereof. In an aspect, a disclosed PRKCA can comprise the amino acid sequence set forth in SEQ ID NO:55 or a fragment thereof. In an aspect, a disclosed PRKCA can comprise a sequence having at least 70%, at least 75%, at least 80%, at least 85%, at least 90%, or at least 95% identity to the sequence set forth in SEQ ID NO:55 or a fragment thereof. In an aspect, a disclosed PRKCA can comprise the amino acid sequence set forth in SEQ ID NO:56 or a fragment thereof. In an aspect, a disclosed PRKCA can comprise a sequence having at least 70%, at least 75%, at least 80%, at least 85%, at least 90%, or at least 95% identity to the sequence set forth in SEQ ID NO:56 or a fragment thereof. In an aspect, a disclosed SYT1 can comprise the amino acid sequence set forth in SEQ ID NO:57 or a fragment thereof. In an aspect, a disclosed SYT1 can comprise a sequence having at least 70%, at least 75%, at least 80%, at least 85%, at least 90%, or at least 95% identity to the sequence set forth in SEQ ID NO:57 or a fragment thereof. In an aspect, a disclosed SYT1 can comprise the amino acid sequence set forth in SEQ ID NO:58 or a fragment thereof. In an aspect, a disclosed SYT1 can comprise a sequence having at least 70%, at least 75%, at least 80%, at least 85%, at least 90%, or at least 95% identity to the sequence set forth in SEQ ID NO:58 or a fragment thereof. In an aspect, a disclosed JMJD6 can comprise the amino acid sequence set forth in SEQ ID NO: 59 or a fragment thereof. In an aspect, a disclosed JMJD6 can comprise a sequence having at least 70%, at least 75%, at least 80%, at least 85%, at least 90%, or at least 95% identity to the sequence set forth in SEQ ID NO:59 or a fragment thereof.

| Binding | |
|---|---|
| Domain | Identifying Information |
| ANXA1 | HGNC: 533/NCBI Entrez Gene: 301/Ensembl: ENSG00000135046/ |
| | OMIM: 151690/UniProtKB/Swiss-Prot: P04083 |
| ANXA2 | HGNC: 537/NCBI Entrez Gene: 302/Ensembl: ENSG00000182718/ |
| | OMIM: 151740/UniProtKB/Swiss-Prot: P07355) |
| ANXA3 | HGNC: 541/NCBI Entrez Gene: 306/Ensembl: ENSG00000138772/ |
| | OMIM: 106490/UniProtKB/Swiss-Prot: P12429 |
| ANXA4 | HGNC: 542/NCBI Entrez Gene: 307/Ensembl: ENSG00000196975/ |
| | OMIM: 106491/UniProtKB/Swiss-Prot: P09525 |
| ANXA5 | HGNC: 543/NCBI Entrez Gene: 308/Ensembl: ENSG00000164111/ |
| | OMIM: 131230/UniProtKB/Swiss-Prot: P08758 |
| ANXA6 | HGNC: 544/NCBI Entrez Gene: 309/Ensembl: ENSG00000197043/ |
| | OMIM: 114070/UniProtKB/Swiss-Prot: P08133 |
| ANXA7 | HGNC: 545/NCBI Entrez Gene: 310/Ensembl: ENSG00000138279/ |
| | OMIM: 186360/UniProtKB/Swiss-Prot: P20073 |
| ANXA8 | HGNC: 546/NCBI Entrez Gene: 653145/Ensembl: ENSG00000265190/ |
| | OMIM: 602396/UniProtKB/Swiss-Prot: P13928 |
| ANX8L1 | HGNC: 23334/NCBI Entrez Gene: 728113/Ensembl: ENSG00000264230/ |
| | UniProtKB/Swiss-Prot: Q5VT79 |
| ANXA9 | HGNC: 547/NCBI Entrez Gene: 8416/Ensembl: ENSG00000143412/ |
| | OMIM: 603319/UniProtKB/Swiss-Prot: 076027 |
| ANXA10 | HGNC: 534/NCBI Entrez Gene: 11199/Ensembl: ENSG00000109511/ |
| | OMIM: 608008/UniProtKB/Swiss-Prot: Q9UJ72 |
| ANXA11 | HGNC: 535/NCBI Entrez Gene: 311/Ensembl: ENSG00000122359/ |
| | OMIM: 602572/UniProtKB/Swiss-Prot: P50995 |
| ANXA13 | HGNC: 536/NCBI Entrez Gene: 312/Ensembl: ENSG00000104537/ |
| | OMIM: 602573/UniProtKB/Swiss-rot: P27216 |
| F2 | HGNC: 3535/NCBI Entrez Gene: 2147/Ensembl: ENSG00000180210/ |
| | OMIM: 176930/UniProtKB/Swiss-Prot: P00734) |
| F7 | (HGNC: 3544/NCBI Entrez Gene: 2155/Ensembl: ENSG00000057593/ |
| | OMIM: 613878/UniProtKB/Swiss-Prot: P08709) |
| F9 | (HGNC: 3551/NCBI Entrez Gene: 2158/Ensembl: ENSG00000101981/ |
| | OMIM: 300746/UniProtKB/Swiss-Prot: P00740) |
| F10 | (HGNC: 3528/NCBI Entrez Gene: 2159/Ensembl: ENSG00000126218/ |
| | OMIM: 613872/UniProtKB/Swiss-Prot: P00742) |
| AGER/ | (HGNC: 320 NCBI/Entrez Gene: 177/Ensembl: ENSG00000204305/ |
| RAGE | OMIM: 600214/UniProtKB/Swiss-Prot: Q15109 |
| GAS6 | HGNC: 4168 NCBI/Entrez Gene: 2621/Ensembl: ENSG00000183087/ |
| | OMIM: 600441/UniProtKB/Swiss-Prot: Q14393 |
| MFGE8 | HGNC: 7036 NCBI Entrez Gene: 4240/Ensembl: ENSG00000140545/ |
| | OMIM: 602281/UniProtKB/Swiss-Prot: Q08431 |
| AGCR/ | HGNC: 943/NCBI Entrez Gene: 575/Ensembl: ENSG00000181790/ |
| BAI1 | OMIM: 602682/UniProtKB/Swiss-Prot: O14514) |
| PROS | HGNC: 9456 NCBI Entrez Gene: 5627 Ensembl: ENSG00000184500 |
| | OMIM: 176880/UniProtKB/Swiss-Prot: P07225 |
| HAVCR1 | HGNC: 17866/NCBI Entrez Gene: 26762/Ensembl: ENSG00000113249/ |
| | OMIM: 606518/UniProtKB/Swiss-Prot: Q96D42 |
| HAVCR1 | HGNC: 18437/NCBI Entrez Gene: 84868 Ensembl: ENSG00000135077/ |
| | OMIM: 606652/UniProtKB/Swiss-Prot: Q8TDQ0 |
| TIMD4 | HGNC: 25132/NCBI Entrez Gene: 91937 Ensembl: ENSG00000145850/ |
| | OMIM: 610096/UniProtKB/Swiss-Prot: Q96H15 |
| PKCα | HGNC: 9393/NCBI Entrez Gene: 5578 Ensembl: ENSG00000154229/ |
| | OMIM: 176960/UniProtKB/Swiss-Prot: P17252 |
| SYT1 | HGNC: 11509/NCBI Entrez Gene: 6857 Ensembl: ENSG00000067715/ |
| | OMIM: 185605 UniProtKB/Swiss-Prot: P21579 |
| JMJD6 | HGNC: 19355/NCBI Entrez Gene: 23210 Ensembl: ENSG00000070495 |
| | OMIM: 604914 UniProtKB/Swiss-Prot: Q6NYC1 |
| APO-H | HGNC: 616/NCBI Entrez Gene: 350/Ensembl: ENSG00000091583/ |
| | OMIM: 138700/UniProtKB/Swiss-Prot: P02749 |
| STAB1 | HGNC: 18628/NCBI Entrez Gene: 23166/Ensembl: ENSG00000010327/ |
| | OMIM: 608560/UniProtKB/Swiss-Prot: Q9NY15 |
| STAB2 | HGNC: 18629/NCBI Entrez Gene: 55576/Ensembl: ENSG00000136011/ |
| | OMIM: 608561/UniProtKB/Swiss-Prot: Q8WWQ8 |

In an aspect, a disclosed PS binding domain can comprise the single-chain variable domain of bavituximab. In an aspect, a disclosed PS binding domain can comprise the sequence set forth in SEQ ID NO:73 or a fragment thereof. In an aspect, a disclosed PS binding chain can comprise a sequence having at least 70%, at least 75%, at least 80%, at least 85%, at least 90%, or at least 95% identity to the sequence set forth in SEQ ID NO:73 or a fragment thereof. In an aspect, a disclosed PS binding domain can comprise the single-chain variable domain of PGN632. In an aspect, a single-chain variable domain of PGN632 can comprise a γ1 heavy chain and a λ light chain. In an aspect, a single-chain variable domain of PGN632 can bind to cardiolipin/PS. In an aspect, a disclosed PS binding domain can comprise the single-chain variable domain of P1. In an aspect, a single-chain variable domain of P1 can comprise a γ1 heavy chain and a λ light chain. In an aspect, a single-chain variable domain of P1 can bind to cardiolipin/PS. In an aspect, a disclosed PS binding domain can comprise the single-chain variable domain of IS4. In an aspect, a single-chain variable domain of IS4 can comprise a 13 VH1 heavy chain and a λ Vλ2 light chain. In an aspect, a single-chain variable domain of IS4 can bind to cardiolipin/PS. In an aspect, a disclosed PS binding domain can comprise the single-chain variable domain of CL1. In an aspect, a single-chain variable domain of CLL can comprise a γ3 VH1 heavy chain and a λ Vλ3 light chain. In an aspect, a single-chain variable domain of IS4 can bind to cardiolipin/PS. In an aspect, PGN632, P1, IS4, and CLL are described in Moody et al. (2010) J. Exp. Med. 207 (4): 763-776, which is incorporated herein by reference for its teachings of these antibodies and their characteristics.

In an aspect, a disclosed antigen binding domain can be a scFV and wherein the scFV can comprise a linker. In an aspect, a disclosed linker can join the V_{H} and V_{L} regions of the ScFv.

In an aspect, a disclosed chimeric antigen receptor can further comprise a spacer domain between the extracellular domain and the transmembrane domain. In an aspect, a disclosed spacer domain can comprise an immunoglobulin hinge region, an extracellular region of a type 1 membrane proteins, a part or all of an immunoglobulin constant region, or any combination thereof.

In an aspect, a disclosed spacer domain can comprise a hinge region. In an aspect, a disclosed hinge region can comprise a hinge region of CD8a, CD28, IgG1, IgG2, IgG3, IgG4, IgA, IgD, or any combination thereof. In an aspect, a disclosed CD8a hinge domain can comprise the sequence set forth in SEQ ID NO:09 or a fragment thereof. In an aspect, a disclosed hinge region can be from or can be derived from CD2, CD3 delta, CD3 epsilon, CD3 gamma, CD4, CD7, CD8a, CD813, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA1-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, Toll ligand receptor, or any combination thereof, or any fragment/truncation thereof, or any combination of fragments/truncations thereof.

In an aspect, a disclosed chimeric antigen receptor can further comprise a transmembrane domain. In an aspect, a disclosed transmembrane can further comprise a transmembrane domain of CD2, CD3γ, CD3ε, CD3δ, CD3ξ, CD4, CD8, CD25, CD27, CD28, CD40, CD79A, CD79B, CD79B, CD80, CD86, CD95 (FAS), CD134 (OX40), CD137 (4-1BB), CD154, CD278 (ICOS), TCRα, TCRβ, NKG2D, 2B4, or any combination thereof.

In an aspect, a disclosed CD28 transmembrane domain can comprise the sequence set forth in SEQ ID NO:12 or a fragment thereof. In an aspect, a disclosed transmembrane domain can comprise a CD28 transmembrane domain or a truncated CD28 domain.

In an aspect, a disclosed transmembrane domain can be from or can be derived from the alpha, beta or zeta chain of a T-cell receptor, 2B4, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CDS, CEACAM1, CRT AM, cytokine receptor, DAP-10, DAP-12, DNAMI (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a combination thereof, or a fragment/truncation thereof, or a combination of fragments and/or truncations thereof.

In an aspect, a disclosed intracellular domain can comprise the intracellular domain of CD28. In an aspect, a disclosed intracellular domain of CD28 can comprise the sequence set froth in SEQ ID NO:11 or a fragment thereof.

In an aspect, a disclosed intracellular domain can comprise one or more immunoreceptor tyrosine-based activation domains (ITAMs). In an aspect, a disclosed ITAM can comprise the signaling domain of DAP10, DAP12, TCRζ, FcRγ, FcRβ, CD3γ, CD3ξ, CD3ε, CD3δ, CD3ξ, CD5, CD22, CD66d, CD79a, CD79b, CD278 (ICOS), or any combination thereof.

In an aspect, a disclosed intracellular domain can further comprise one or more co-stimulatory domains. In an aspect, a disclosed co-stimulatory domain can comprise the signaling domain of CD27, CD28, 4-1BB, OX40, CD30, CD40, PD-1, ICOS (CD278), LFA-1, CD2, CD7 LIGHT, NKG2C, B7-H3, or any combination thereof. In an aspect, a disclosed costimulatory molecule can comprise 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha, beta, delta, epsilon, gamma, zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAMI (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

In an aspect of a disclosed chimeric antigen receptor, a disclosed intracellular domain can comprise an ITAM and one or more co-stimulatory domains, wherein the ITAM can comprise the signaling domain of CD3γ, CD3ξ, CD3ε, CD3δ, CD3ξ, CD5, CD22, CD79a, CD278 (ICOS), or any combination thereof, and wherein the co-stimulatory domains can comprises the signaling domain of CD27, CD28, 4-1BB, OX40, CD30, CD40, PD-1, ICOS, LFA-1, CD2, CD7 LIGHT, NKG2C, B7-H3, or any combination thereof.

In an aspect of a disclosed chimeric antigen receptor, a disclosed intracellular domain can comprise one or more ITAMs and one or more co-stimulatory domains, wherein the ITAMs can comprise the signaling domain of CD3γ, CD3ξ, CD3ε, CD3δ, CD3ξ, CD5, CD22, CD79a, CD278 (ICOS), or any combination thereof, and wherein the co-stimulatory domains can comprise the signaling domain of CD27, CD28, 4-1BB, OX40, CD30, CD40, PD-1, ICOS, LFA-1, CD2, CD7 LIGHT, NKG2C, B7-H3, or any combination thereof.

In an aspect, the disclosed two co-stimulatory domains can comprise two of the same signaling domain. In an aspect, the disclosed two co-stimulatory domains can comprise two different signaling domains. In an aspect, a disclosed 4-1BB signaling domain can comprise the sequence set forth in SEQ ID NO:13 or a fragment thereof. In an aspect, a disclosed CD3g signaling domain comprises the sequence set forth in SEQ ID NO:14 or a fragment thereof.

In an aspect, a disclosed co-stimulatory domain can comprise CD28. In an aspect, a disclosed CD28 co-stimulatory domain can demonstrate one or more functional aspects: (i) lower persistence and differentiation towards effector memory phenotype compared to 41BB second-generation CARS, (ii) more prone to tonic signaling and causes early exhaustion, (iii) imparts resistance to Tregs in-vitro, in-vivo models however suggested that CD28 co-stimulation causes increased infiltration of Tregs and were less effective against tumors in presence of Tregs, (iv) resistant to CTLA4 inhibition, (v) faster and higher signaling intensity, (vi) does not alter scFv “affinity ceiling”-affinity beyond which IFNγ, IL2 secretion and cytotoxicity do not increase, or (vii) any combination thereof.

In an aspect, a disclosed co-stimulatory domain can comprise 4-1BB. In an aspect, a disclosed 4-1BB co-stimulatory domain can demonstrate one or more functional aspects: (i) greater persistence and differentiation towards central memory phenotype compared to CD28 second-generation CARs, (ii) can reduce tonic signaling at optimal expression levels and decrease exhaustion, (iii) slower and less intense signaling, or (iv) any combination thereof.

In an aspect of a chimeric antigen receptor, a disclosed intracellular domain can further comprise a self-cleaving peptide. In an aspect, a disclosed self-cleaving peptide can comprise a T2A, a P2A, a E2A, or F2A peptide. In an aspect, a GSG linker can be added to the N-terminus of a disclosed 2A peptide. In an aspect, a disclosed P2A peptide can comprise the sequence set forth in SEQ ID NO:15 or SEQ ID NO: 102 or a fragment thereof. In an aspect, a disclosed T2A peptide can comprise the sequence set forth in SEQ ID NO: 103 or SEQ ID NO: 104 or a fragment thereof. In an aspect, a disclosed E2A peptide can comprise the sequence set forth in SEQ ID NO: 105 or SEQ ID NO:106 or a fragment thereof. In an aspect, a disclosed F2A peptide can comprise the sequence set forth in SEQ ID NO:107 or SEQ ID NO:108 or a fragment thereof.

In an aspect of a chimeric antigen receptor, a disclosed intracellular domain can further comprise a sequence encoding an EGFR domain or a truncated EGFR domain. In an aspect, a disclosed EGFR domain can comprise the sequence set forth in SEQ ID NO:17 or a fragment thereof. In an aspect, a disclosed truncated EGFR domain can comprise the sequence set forth in SEQ ID NO: 18 or a fragment thereof.

In an aspect of a chimeric antigen receptor, a disclosed intracellular domain can further comprise a sequence encoding a self-cleaving peptide, a signal peptide, and an EGFR domain or truncated EGFR domain, or any combination thereof. In an aspect, a disclosed intracellular domain comprising a disclosed self-cleaving peptide, the signal peptide, and the truncated EGFR domain can comprise the sequence set forth in SEQ ID NO:04 or a fragment thereof. In an aspect, a disclosed intracellular domain comprising a disclosed self-cleaving peptide, the signal peptide, and the truncated EGFR domain can comprise a sequence having at least 70%, at least 75%, at least 80%, at least 85%, at least 90%, or at least 95% identity to the sequence set forth in SEQ ID NO: 04.

In an aspect, a disclosed CAR can comprise a first-generation CAR, a second-generation CAR, a third-generation CAR, a fourth-generation CAR, or a fifth-generation CAR.

In an aspect, a disclosed CAR can comprise an amino acid sequence having the sequence set forth in SEQ ID NO:01, SEQ ID NO:05, or fragment thereof. In an aspect, a disclosed CAR can comprise an amino acid sequence having the sequence set forth in SEQ ID NO:02, SEQ ID NO: 06, or fragment thereof. In an aspect, a disclosed CAR can comprise an amino acid sequence having the sequence set forth in SEQ ID NO:03, SEQ ID NO:07, or fragment thereof. In an aspect, a disclosed CAR can comprise an amino acid sequence having at least 70%, at least 75%, at least 80%, at least 85%, at least 90%, or at least 95% identity to the sequence set forth in SEQ ID NO: 01, SEQ ID NO:05, or fragment thereof. In an aspect, a disclosed CAR can comprise an amino acid sequence having at least 70%, at least 75%, at least 80%, at least 85%, at least 90%, or at least 95% identity to the sequence set forth in SEQ ID NO:02, SEQ ID NO:06, or fragment thereof. In an aspect, a disclosed CAR can comprise an amino acid sequence having at least 70%, at least 75%, at least 80%, at least 85%, at least 90%, or at least 95% identity to the sequence set forth in SEQ ID NO:03, SEQ ID NO:07, or fragment thereof.

In an aspect, a disclosed CAR can stimulate an effector cell mediated immune modulator response to PS-expressing tumor cells. In an aspect, a disclosed CAR can induce a tumor reducing immune response. In an aspect, a disclosed CAR can induce phagocytosis of cancer cells in the subject. In an aspect, a disclosed CAR can cross-prime an anti-tumor T cell response. In an aspect, a disclosed CAR can induce a tumor eliminating immune response. In an aspect, a disclosed CAR can treat cancer.

Disclosed herein is a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein s a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor-specific antigen or fragment thereof.

In an aspect, a disclosed CAR can be introduced to T cells and/or NK and/or macrophages or any other immune system. In an aspect, a disclosed CAR can be used to activated one or more types of immune cells (e.g., naive T cells, central memory T cells, effector memory T cells, NK cells or combination thereof) upon antigen binding.

### 2. Isolated Nucleic Acid Molecules

Disclosed herein is an isolated nucleic acid molecule comprising a nucleic acid sequence encoding any disclosed CAR. Disclosed herein is an isolated nucleic acid molecule comprising a nucleic acid sequence encoding a CAR specific for phosphatidylserine (PS). Disclosed herein is an isolated nucleic acid molecule comprising a nucleic acid sequence encoding a CAR comprising an extracellular domain comprising an antigen binding domain, a transmembrane domain, and an intracellular domain comprising one or more immunostimulatory domains.

Disclosed herein is an isolated nucleic acid molecule comprising a nucleic acid sequence encoding a CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein is an isolated nucleic acid molecule comprising a nucleic acid sequence encoding a CAR comprising from N-terminus to C-terminus a signal peptide; an antigen binding domain; a hinge domain; a transmembrane domain; an intracellular domain, a cleavage signal; a signal peptide; and a tumor-specific antigen or fragment thereof. Disclosed herein is an isolated nucleic acid molecule comprising a nucleic acid sequence encoding a phosphatidylserine-specific CAR comprising an extracellular domain comprising an antigen binding domain, a transmembrane domain, and an intracellular domain comprising one or more immunostimulatory domains.

Disclosed herein is an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains.

Disclosed herein is an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof.

Disclosed herein is an isolated nucleic acid molecule comprising a nucleic acid sequence encoding a CAR comprising the sequence set forth in SEQ ID NO:01, SEQ ID NO:02, or SEQ ID NO: 03. Disclosed herein is an isolated nucleic acid molecule comprising a nucleic acid sequence encoding a CAR comprising the sequence set forth in SEQ ID NO:05, SEQ ID NO:06, or SEQ ID NO: 07. Disclosed herein is an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:01, SEQ ID NO: 02, or SEQ ID NO:03. Disclosed herein is an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO: 05, SEQ ID NO:06, or SEQ ID NO:07.

In an aspect of a disclosed isolated nucleic acid molecule encoding a phosphatidylserine-specific CAR, the extracellular domain can further comprise a signal peptide. In an aspect, a disclosed signal peptide can comprise a CD8 signal peptide. In an aspect, a disclosed CD8 signal peptide can comprise the sequence set forth in SEQ ID NO:08 or a fragment thereof. In an aspect, a disclosed CD8 signal peptide can comprise a sequence having at least 70%, at least 75%, at least 80%, at least 85%, at least 90%, or at least 95% identity to the sequence set forth in SEQ ID NO:08 or a fragment thereof.

In an aspect of a disclosed isolated nucleic acid molecule encoding a phosphatidylserine-specific CAR, the extracellular domain can further comprise a phosphatidylserine (PS) binding domain. In an aspect, a disclosed PS binding domain can comprise Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect of a disclosed isolated nucleic acid molecule, a disclosed encoded ANXA1 can comprise the amino acid sequence set forth in SEQ ID NO:19 or a fragment thereof. In an aspect, a disclosed encoded ANXA1 signal peptide can comprise a sequence having at least 70%, at least 75%, at least 80%, at least 85%, at least 90%, or at least 95% identity to the sequence set forth in SEQ ID NO:19 or a fragment thereof. In an aspect, a disclosed encoded ANXA1 can comprise the amino acid sequence set forth in SEQ ID NO:20 or a fragment thereof. In an aspect, a disclosed encoded ANXA1 signal peptide can comprise a sequence having at least 70%, at least 75%, at least 80%, at least 85%, at least 90%, or at least 95% identity to the sequence set forth in SEQ ID NO:20 or a fragment thereof. In an aspect, a disclosed encoded ANXA2 can comprise the amino acid sequence set forth in SEQ ID NO:21 or a fragment thereof. In an aspect, a disclosed encoded ANXA2 signal peptide can comprise a sequence having at least 70%, at least 75%, at least 80%, at least 85%, at least 90%, or at least 95% identity to the sequence set forth in SEQ ID NO: 21 or a fragment thereof. In an aspect, a disclosed encoded ANXA3 can comprise the amino acid sequence set forth in SEQ ID NO:22 or a fragment thereof. In an aspect, a disclosed encoded ANXA3 signal peptide can comprise a sequence having at least 70%, at least 75%, at least 80%, at least 85%, at least 90%, or at least 95% identity to the sequence set forth in SEQ ID NO:22 or a fragment thereof. In an aspect, a disclosed encoded ANXA4 can comprise the amino acid sequence set forth in SEQ ID NO:23 or a fragment thereof. In an aspect, a disclosed encoded ANXA4 signal peptide can comprise a sequence having at least 70%, at least 75%, at least 80%, at least 85%, at least 90%, or at least 95% identity to the sequence set forth in SEQ ID NO:23 or a fragment thereof. In an aspect, a disclosed encoded ANXA5 can comprise the amino acid sequence set forth in SEQ ID NO:24 or a fragment thereof. In an aspect, a disclosed encoded ANXA5 signal peptide can comprise a sequence having at least 70%, at least 75%, at least 80%, at least 85%, at least 90%, or at least 95% identity to the sequence set forth in SEQ ID NO:24 or a fragment thereof. In an aspect, a disclosed encoded ANXA6 can comprise the amino acid sequence set forth in SEQ ID NO:25 or a fragment thereof. In an aspect, a disclosed encoded ANXA6 signal peptide can comprise a sequence having at least 70%, at least 75%, at least 80%, at least 85%, at least 90%, or at least 95% identity to the sequence set forth in SEQ ID NO:25 or a fragment thereof. In an aspect, a disclosed encoded ANXA7 can comprise the amino acid sequence set forth in SEQ ID NO:26 or a fragment thereof. In an aspect, a disclosed encoded ANXA7 signal peptide can comprise a sequence having at least 70%, at least 75%, at least 80%, at least 85%, at least 90%, or at least 95% identity to the sequence set forth in SEQ ID NO:26 or a fragment thereof. In an aspect, a disclosed encoded ANXA8 can comprise the amino acid sequence set forth in SEQ ID NO:27 or a fragment thereof. In an aspect, a disclosed encoded ANXA8 signal peptide can comprise a sequence having at least 70%, at least 75%, at least 80%, at least 85%, at least 90%, or at least 95% identity to the sequence set forth in SEQ ID NO:27 or a fragment thereof. In an aspect, a disclosed encoded ANXA8L can comprise the amino acid sequence set forth in SEQ ID NO:28 or a fragment thereof. In an aspect, a disclosed encoded ANXA8L signal peptide can comprise a sequence having at least 70%, at least 75%, at least 80%, at least 85%, at least 90%, or at least 95% identity to the sequence set forth in SEQ ID NO:28 or a fragment thereof. In an aspect, a disclosed encoded ANXA9 can comprise the amino acid sequence set forth in SEQ ID NO:29 or a fragment thereof. In an aspect, a disclosed encoded ANXA9 signal peptide can comprise a sequence having at least 70%, at least 75%, at least 80%, at least 85%, at least 90%, or at least 95% identity to the sequence set forth in SEQ ID NO:29 or a fragment thereof. In an aspect, a disclosed encoded ANXA10 can comprise the amino acid sequence set forth in SEQ ID NO:30 or a fragment thereof. In an aspect, a disclosed encoded ANXA10 signal peptide can comprise a sequence having at least 70%, at least 75%, at least 80%, at least 85%, at least 90%, or at least 95% identity to the sequence set forth in SEQ ID NO:30 or a fragment thereof. In an aspect, a disclosed encoded ANXA11 can comprise the amino acid sequence set forth in SEQ ID NO:31 or a fragment thereof. In an aspect, a disclosed encoded ANXA11 signal peptide can comprise a sequence having at least 70%, at least 75%, at least 80%, at least 85%, at least 90%, or at least 95% identity to the sequence set forth in SEQ ID NO:31 or a fragment thereof. In an aspect, a disclosed encoded ANXA13 can comprise the amino acid sequence set forth in SEQ ID NO:32 or a fragment thereof. In an aspect, a disclosed encoded ANXA13 signal peptide can comprise a sequence having at least 70%, at least 75%, at least 80%, at least 85%, at least 90%, or at least 95% identity to the sequence set forth in SEQ ID NO:32 or a fragment thereof.

In an aspect of a disclosed isolated nucleic acid molecule, a disclosed encoded ADGRB1 can comprise the amino acid sequence set forth in SEQ ID NO:33 or a fragment thereof. In an aspect, a disclosed encoded ADGRB1 signal peptide can comprise a sequence having at least 70%, at least 75%, at least 80%, at least 85%, at least 90%, or at least 95% identity to the sequence set forth in SEQ ID NO:33 or a fragment thereof. In an aspect, a disclosed encoded ADGRB1 can comprise the amino acid sequence set forth in SEQ ID NO:34 or a fragment thereof. In an aspect, a disclosed encoded ADGRB1 signal peptide can comprise a sequence having at least 70%, at least 75%, at least 80%, at least 85%, at least 90%, or at least 95% identity to the sequence set forth in SEQ ID NO:34 or a fragment thereof. In an aspect, a disclosed encoded APO-H can comprise the amino acid sequence set forth in SEQ ID NO:35 or a fragment thereof. In an aspect, a disclosed encoded APO-H can comprise a sequence having at least 70%, at least 75%, at least 80%, at least 85%, at least 90%, or at least 95% identity to the sequence set forth in SEQ ID NO:35 or a fragment thereof. In an aspect, a disclosed encoded F2 can comprise the amino acid sequence set forth in SEQ ID NO:36 or a fragment thereof. In an aspect, a disclosed encoded F2 can comprise a sequence having at least 70%, at least 75%, at least 80%, at least 85%, at least 90%, or at least 95% identity to the sequence set forth in SEQ ID NO:36 or a fragment thereof. In an aspect, a disclosed encoded F7 can comprise the amino acid sequence set forth in SEQ ID NO:37 or a fragment thereof. In an aspect, a disclosed encoded F7 can comprise a sequence having at least 70%, at least 75%, at least 80%, at least 85%, at least 90%, or at least 95% identity to the sequence set forth in SEQ ID NO:37 or a fragment thereof. In an aspect, a disclosed encoded F9 can comprise the amino acid sequence set forth in SEQ ID NO:38 or a fragment thereof. In an aspect, a disclosed encoded F9 can comprise a sequence having at least 70%, at least 75%, at least 80%, at least 85%, at least 90%, or at least 95% identity to the sequence set forth in SEQ ID NO:38 or a fragment thereof. In an aspect, a disclosed encoded F10 can comprise the amino acid sequence set forth in SEQ ID NO:39 or a fragment thereof. In an aspect, a disclosed encoded F10 can comprise a sequence having at least 70%, at least 75%, at least 80%, at least 85%, at least 90%, or at least 95% identity to the sequence set forth in SEQ ID NO:39 or a fragment thereof. In an aspect, a disclosed encoded GAS6 can comprise the amino acid sequence set forth in SEQ ID NO: 40 or a fragment thereof. In an aspect, a disclosed encoded GAS6 an comprise a sequence having at least 70%, at least 75%, at least 80%, at least 85%, at least 90%, or at least 95% identity to the sequence set forth in SEQ ID NO:40 or a fragment thereof. In an aspect, a disclosed encoded MFGE8 can comprise the amino acid sequence set forth in SEQ ID NO:41 or a fragment thereof. In an aspect, a disclosed encoded MFGE8 can comprise a sequence having at least 70%, at least 75%, at least 80%, at least 85%, at least 90%, or at least 95% identity to the sequence set forth in SEQ ID NO:41 or a fragment thereof. In an aspect, a disclosed encoded AGER can comprise the amino acid sequence set forth in SEQ ID NO:42 or a fragment thereof. In an aspect, a disclosed encoded AGER can comprise a sequence having at least 70%, at least 75%, at least 80%, at least 85%, at least 90%, or at least 95% identity to the sequence set forth in SEQ ID NO:42 or a fragment thereof. In an aspect, a disclosed encoded AGER can comprise the amino acid sequence set forth in SEQ ID NO:43 or a fragment thereof. In an aspect, a disclosed encoded AGER can comprise a sequence having at least 70%, at least 75%, at least 80%, at least 85%, at least 90%, or at least 95% identity to the sequence set forth in SEQ ID NO:43 or a fragment thereof. In an aspect, a disclosed encoded PROS1 can comprise the amino acid sequence set forth in SEQ ID NO:44 or a fragment thereof. In an aspect, a disclosed encoded PROS1 can comprise a sequence having at least 70%, at least 75%, at least 80%, at least 85%, at least 90%, or at least 95% identity to the sequence set forth in SEQ ID NO:44 or a fragment thereof. In an aspect, a disclosed encoded STAB1 can comprise the amino acid sequence set forth in SEQ ID NO:45 or a fragment thereof. In an aspect, a disclosed encoded STAB1 can comprise a sequence having at least 70%, at least 75%, at least 80%, at least 85%, at least 90%, or at least 95% identity to the sequence set forth in SEQ ID NO:45 or a fragment thereof. In an aspect, a disclosed encoded STAB1 can comprise the amino acid sequence set forth in SEQ ID NO:46 or a fragment thereof. In an aspect, a disclosed encoded STAB1 can comprise a sequence having at least 70%, at least 75%, at least 80%, at least 85%, at least 90%, or at least 95% identity to the sequence set forth in SEQ ID NO:46 or a fragment thereof. In an aspect, a disclosed encoded STAB2 can comprise the amino acid sequence set forth in SEQ ID NO:47 or a fragment thereof. In an aspect, a disclosed encoded STAB2 can comprise a sequence having at least 70%, at least 75%, at least 80%, at least 85%, at least 90%, or at least 95% identity to the sequence set forth in SEQ ID NO:47 or a fragment thereof. In an aspect, a disclosed encoded STAB2 can comprise the amino acid sequence set forth in SEQ ID NO:48 or a fragment thereof. In an aspect, a disclosed encoded STAB2 can comprise a sequence having at least 70%, at least 75%, at least 80%, at least 85%, at least 90%, or at least 95% identity to the sequence set forth in SEQ ID NO:48 or a fragment thereof. In an aspect, a disclosed encoded HAVCR1 can comprise the amino acid sequence set forth in SEQ ID NO:49 or a fragment thereof. In an aspect, a disclosed encoded HAVCR1 can comprise a sequence having at least 70%, at least 75%, at least 80%, at least 85%, at least 90%, or at least 95% identity to the sequence set forth in SEQ ID NO:49 or a fragment thereof. In an aspect, a disclosed encoded HAVCR1 can comprise the amino acid sequence set forth in SEQ ID NO:50 or a fragment thereof. In an aspect, a disclosed encoded HAVCR1 can comprise a sequence having at least 70%, at least 75%, at least 80%, at least 85%, at least 90%, or at least 95% identity to the sequence set forth in SEQ ID NO:50 or a fragment thereof. In an aspect, a disclosed encoded HAVCR2 can comprise the amino acid sequence set forth in SEQ ID NO:51 or a fragment thereof. In an aspect, a disclosed encoded HAVCR2 can comprise a sequence having at least 70%, at least 75%, at least 80%, at least 85%, at least 90%, or at least 95% identity to the sequence set forth in SEQ ID NO:51 or a fragment thereof. In an aspect, a disclosed encoded HAVCR2 can comprise the amino acid sequence set forth in SEQ ID NO:52 or a fragment thereof. In an aspect, a disclosed encoded HAVCR2 can comprise a sequence having at least 70%, at least 75%, at least 80%, at least 85%, at least 90%, or at least 95% identity to the sequence set forth in SEQ ID NO:52 or a fragment thereof. In an aspect, a disclosed encoded TIMD4 can comprise the amino acid sequence set forth in SEQ ID NO: 53 or a fragment thereof. In an aspect, a disclosed encoded TIMD4 can comprise a sequence having at least 70%, at least 75%, at least 80%, at least 85%, at least 90%, or at least 95% identity to the sequence set forth in SEQ ID NO:53 or a fragment thereof. In an aspect, a disclosed encoded TIMD4 can comprise the amino acid sequence set forth in SEQ ID NO:54 or a fragment thereof. In an aspect, a disclosed encoded TIMD4 can comprise a sequence having at least 70%, at least 75%, at least 80%, at least 85%, at least 90%, or at least 95% identity to the sequence set forth in SEQ ID NO:54 or a fragment thereof. In an aspect, a disclosed encoded PRKCA can comprise the amino acid sequence set forth in SEQ ID NO:55 or a fragment thereof. In an aspect, a disclosed encoded PRKCA can comprise a sequence having at least 70%, at least 75%, at least 80%, at least 85%, at least 90%, or at least 95% identity to the sequence set forth in SEQ ID NO:55 or a fragment thereof. In an aspect, a disclosed encoded PRKCA can comprise the amino acid sequence set forth in SEQ ID NO:56 or a fragment thereof. In an aspect, a disclosed encoded PRKCA can comprise a sequence having at least 70%, at least 75%, at least 80%, at least 85%, at least 90%, or at least 95% identity to the sequence set forth in SEQ ID NO:56 or a fragment thereof. In an aspect, a disclosed encoded SYT1 can comprise the amino acid sequence set forth in SEQ ID NO: 57 or a fragment thereof. In an aspect, a disclosed encoded SYT1 can comprise a sequence having at least 70%, at least 75%, at least 80%, at least 85%, at least 90%, or at least 95% identity to the sequence set forth in SEQ ID NO:57 or a fragment thereof. In an aspect, a disclosed encoded SYT1 can comprise the amino acid sequence set forth in SEQ ID NO:58 or a fragment thereof. In an aspect, a disclosed encoded SYT1 can comprise a sequence having at least 70%, at least 75%, at least 80%, at least 85%, at least 90%, or at least 95% identity to the sequence set forth in SEQ ID NO:58 or a fragment thereof. In an aspect, a disclosed encoded JMJD6 can comprise the amino acid sequence set forth in SEQ ID NO:59 or a fragment thereof. In an aspect, a disclosed encoded JMJD6 can comprise a sequence having at least 70%, at least 75%, at least 80%, at least 85%, at least 90%, or at least 95% identity to the sequence set forth in SEQ ID NO:59 or a fragment thereof.

In an aspect of a disclosed isolated nucleic acid molecule, a disclosed encoded PS binding domain can comprise the single-chain variable domain of bavituximab. In an aspect, a disclosed encoded PS binding domain can comprise the sequence set forth in SEQ ID NO:73 or a fragment thereof. In an aspect, a disclosed encoded PS binding chain can comprise a sequence having at least 70%, at least 75%, at least 80%, at least 85%, at least 90%, or at least 95% identity to the sequence set forth in SEQ ID NO:73 or a fragment thereof. In an aspect, a disclosed encoded PS binding domain can comprise the single-chain variable domain of PGN632. In an aspect, a single-chain variable domain of PGN632 can comprise a γ1 heavy chain and a 2 light chain. In an aspect, a single-chain variable domain of PGN632 can bind to cardiolipin/PS. In an aspect, a disclosed encoded PS binding domain can comprise the single-chain variable domain of P1. In an aspect, a single-chain variable domain of P1 can comprise a γ1 heavy chain and a λ light chain. In an aspect, a single-chain variable domain of P1 can bind to cardiolipin/PS. In an aspect, a disclosed encoded PS binding domain can comprise the single-chain variable domain of IS4. In an aspect, a single-chain variable domain of IS4 can comprise a γ3 VH1 heavy chain and a λ Vλ2 light chain. In an aspect, a single-chain variable domain of IS4 can bind to cardiolipin/PS. In an aspect, a disclosed encoded PS binding domain can comprise the single-chain variable domain of CL1. In an aspect, a single-chain variable domain of CLL can comprise a γ3 VH1 heavy chain and a λ Vλ3 light chain. In an aspect, a single-chain variable domain of IS4 can bind to cardiolipin/PS.

In an aspect, a disclosed antigen binding domain can be a scFV and wherein the scFV can comprise a linker. In an aspect, a disclosed encoded linker can join the V_{H} and V_{L} regions of the ScFv. In an aspect of a disclosed isolated nucleic acid molecule encoding a phosphatidylserine-specific CAR, the spacer domain can comprise an immunoglobulin hinge region, an extracellular region of a type 1 membrane proteins, a part or all of an immunoglobulin constant region, or any combination thereof.

In an aspect of a disclosed isolated nucleic acid molecule encoding a phosphatidylserine-specific CAR, the spacer domain can comprise a hinge region. In an aspect, a disclosed encoded hinge region can comprise a hinge region of CD8a, CD28, IgG1, IgG2, IgG3, IgG4, IgA, IgD, or any combination thereof. In an aspect, a disclosed CD8a hinge domain can comprise the sequence set forth in SEQ ID NO:09 or a fragment thereof. In an aspect, a disclosed encoded hinge region can be from or can be derived from CD2, CD3 delta, CD3 epsilon, CD3 gamma, CD4, CD7, CD8a, CD813, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA1-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, Toll ligand receptor, or any combination thereof, or any fragment/truncation thereof, or any combination of fragments/truncations thereof.

In an aspect of a disclosed isolated nucleic acid molecule encoding a phosphatidylserine-specific CAR, the transmembrane domain can further comprise a transmembrane domain of CD2, CD3γ, CD3ε, CD3δ, CD3ξ, CD4, CD8, CD25, CD27, CD28, CD40, CD79A, CD79B, CD79B, CD80, CD86, CD95 (FAS), CD134 (OX40), CD137 (4-1BB), CD154, CD278 (ICOS), TCRα, TCRβ, NKG2D, 2B4, or any combination thereof. In an aspect, a disclosed encoded CD28 transmembrane domain can comprise the sequence set forth in SEQ ID NO:12 or a fragment thereof. In an aspect, a disclosed encoded transmembrane domain can comprise a CD28 transmembrane domain or a truncated CD28 domain. In an aspect, a disclosed encoded transmembrane domain can be from or can be derived from the alpha, beta or zeta chain of a T-cell receptor, 2B4, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CDS, CEACAM1, CRT AM, cytokine receptor, DAP-10, DAP-12, DNAMI (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a combination thereof, or a fragment/truncation thereof, or a combination of fragments and/or truncations thereof.

In an aspect of a disclosed isolated nucleic acid molecule encoding a phosphatidylserine-specific CAR, the encoded intracellular domain can comprise the intracellular domain of CD28. In an aspect, a disclosed intracellular domain of CD28 can comprise the sequence set froth in SEQ ID NO: 11 or a fragment thereof. In an aspect, a disclosed encoded intracellular domain can comprise one or more immunoreceptor tyrosine-based activation domains (ITAMs). In an aspect, a disclosed ITAM can comprise the signaling domain of DAP10, DAP12, TCRζ, FcRγ, FcRβ, CD3γ, CD3ξ, CD3ε, CD3δ, CD3ξ, CD5, CD22, CD66d, CD79a, CD79b, CD278 (ICOS), or any combination thereof.

In an aspect, a disclosed encoded intracellular domain can further comprise one or more co-stimulatory domains. In an aspect, a disclosed encoded co-stimulatory domain can comprise the signaling domain of CD27, CD28, 4-1BB, OX40, CD30, CD40, PD-1, ICOS (CD278), LFA-1, CD2, CD7 LIGHT, NKG2C, B7-H3, or any combination thereof. In an aspect, a disclosed encoded costimulatory molecule can comprise 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha, beta, delta, epsilon, gamma, zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAMI (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

In an aspect of a disclosed isolated nucleic acid molecule, a disclosed encoded intracellular domain can comprise an ITAM and one or more co-stimulatory domains, wherein the ITAM can comprise the signaling domain of CD3γ, CD3ξ, CD3ε, CD3δ, CD3ξ, CD5, CD22, CD79a, CD278 (ICOS), or any combination thereof, and wherein the co-stimulatory domains can comprises the signaling domain of CD27, CD28, 4-1BB, OX40, CD30, CD40, PD-1, ICOS, LFA-1, CD2, CD7 LIGHT, NKG2C, B7-H3, or any combination thereof.

In an aspect of a disclosed isolated nucleic acid molecule, a disclosed encoded intracellular domain can comprise one or more ITAMs and one or more co-stimulatory domains, wherein the ITAMs can comprise the signaling domain of CD3γ, CD3ξ, CD3ε, CD3δ, CD3ξ, CD5, CD22, CD79a, CD278 (ICOS), or any combination thereof, and wherein the co-stimulatory domains can comprise the signaling domain of CD27, CD28, 4-1BB, OX40, CD30, CD40, PD-1, ICOS, LFA-1, CD2, CD7 LIGHT, NKG2C, B7-H3, or any combination thereof.

In an aspect, the disclosed two co-stimulatory domains can comprise two of the same signaling domain. In an aspect, the disclosed two co-stimulatory domains can comprise two different signaling domains. In an aspect, a disclosed 4-1BB signaling domain can comprise the sequence set forth in SEQ ID NO:13 or a fragment thereof. In an aspect, a disclosed CD3g signaling domain comprises the sequence set forth in SEQ ID NO:14 or a fragment thereof.

In an aspect of a disclosed isolated nucleic acid molecule, a disclosed encoded co-stimulatory domain can comprise CD28. In an aspect, a disclosed CD28 co-stimulatory domain can demonstrate one or more functional aspects: (i) lower persistence and differentiation towards effector memory phenotype compared to 41BB second-generation CARs, (ii) more prone to tonic signaling and causes early exhaustion, (iii) imparts resistance to Tregs in-vitro, in-vivo models however suggested that CD28 co-stimulation causes increased infiltration of Tregs and were less effective against tumors in presence of Tregs, (iv) resistant to CTLA4 inhibition, (v) raster and higher signaling intensity, (vi) does not alter scFv “affinity ceiling”-affinity beyond which IFNγ, IL2 secretion and cytotoxicity do not increase, or (vii) any combination thereof.

In an aspect of a disclosed isolated nucleic acid molecule, a disclosed encoded co-stimulatory domain can comprise 4-1BB. In an aspect, a disclosed 4-1BB co-stimulatory domain can demonstrate one or more functional aspects: (i) greater persistence and differentiation towards central memory phenotype compared to CD28 second-generation CARs, (ii) can reduce tonic signaling at optimal expression levels and decrease exhaustion, (iii) slower and less intense signaling, or (iv) any combination thereof.

In an aspect of a disclosed isolated nucleic acid molecule, a disclosed encoded intracellular domain can further comprise a self-cleaving peptide. In an aspect, a disclosed self-cleaving peptide can comprise a T2A, a P2A, a E2A, or F2A peptide. In an aspect, a GSG linker can be added to the N-terminus of a disclosed 2A peptide. In an aspect, a disclosed encoded P2A peptide can comprise the sequence set forth in SEQ ID NO:15 or SEQ ID NO:102 or a fragment thereof. In an aspect, a disclosed encoded T2A peptide can comprise the sequence set forth in SEQ ID NO: 103 or SEQ ID NO:104 or a fragment thereof. In an aspect, a disclosed encoded E2A peptide can comprise the sequence set forth in SEQ ID NO: 105 or SEQ ID NO: 106 or a fragment thereof. In an aspect, a disclosed encoded F2A peptide can comprise the sequence set forth in SEQ ID NO: 107 or SEQ ID NO:108 or a fragment thereof.

In an aspect of a disclosed isolated nucleic acid molecule, a disclosed encoded intracellular domain can further comprise a sequence encoding an EGFR domain or a truncated EGFR domain. In an aspect, a disclosed encoded EGFR domain can comprise the sequence set forth in SEQ ID NO: 17 or a fragment thereof. In an aspect, a disclosed encoded truncated EGFR domain can comprise the sequence set forth in SEQ ID NO:18 or a fragment thereof.

In an aspect of a disclosed isolated nucleic acid molecule, a disclosed encoded intracellular domain can further comprise a sequence encoding a self-cleaving peptide, a signal peptide, and an EGFR domain or truncated EGFR domain, or any combination thereof. In an aspect of a disclosed isolated nucleic acid molecule, a disclosed encoded intracellular domain comprising a disclosed self-cleaving peptide, the signal peptide, and the truncated EGFR domain can comprise the sequence set forth in SEQ ID NO:04 or a fragment thereof. In an aspect of a disclosed isolated nucleic acid molecule, a disclosed encoded intracellular domain comprising a disclosed self-cleaving peptide, the signal peptide, and the truncated EGFR domain can comprise a sequence having at least 70%, at least 75%, at least 80%, at least 85%, at least 90%, or at least 95% identity to the sequence set forth in SEQ ID NO:04, In an aspect of a disclosed isolated nucleic acid molecule, a disclosed encoded CAR can comprise a first-generation CAR, a second-generation CAR, a third-generation CAR, a fourth-generation CAR, or a fifth-generation CAR.

In an aspect, a disclosed isolated nucleic acid molecule can encode an amino acid sequence having the sequence set forth in SEQ ID NO:01, SEQ ID NO:05, or fragment thereof. In an aspect, a disclosed isolated nucleic acid molecule can encode an amino acid sequence having the sequence set forth in SEQ ID NO:02, SEQ ID NO:06, or fragment thereof. In an aspect, a disclosed isolated nucleic acid molecule can encode an amino acid sequence having the sequence set forth in SEQ ID NO: 03, SEQ ID NO:07, or fragment thereof. In an aspect, a disclosed isolated nucleic acid molecule can encode an amino acid sequence having at least 70%, at least 75%, at least 80%, at least 85%, at least 90%, or at least 95% identity to the sequence set forth in SEQ ID NO:01, SEQ ID NO: 05, or fragment thereof. In an aspect, a disclosed isolated nucleic acid molecule can encode an amino acid sequence having at least 70%, at least 75%, at least 80%, at least 85%, at least 90%, or at least 95% identity to the sequence set forth in SEQ ID NO:02, SEQ ID NO:06, or fragment thereof. In an aspect, a disclosed isolated nucleic acid molecule can encode an amino acid sequence having at least 70%, at least 75%, at least 80%, at least 85%, at least 90%, or at least 95% identity to the sequence set forth in SEQ ID NO:03, SEQ ID NO:07, or fragment thereof.

Disclosed herein in an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein in an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor-specific antigen or fragment thereof.

In an aspect, a disclosed isolated nucleic acid molecule can comprise a nucleic acid sequence encoding a signal peptide. In an aspect, a disclosed encoded intracellular domain can further comprise a self-cleaving peptide. In an aspect, a disclosed self-cleaving peptide can comprise a T2A, a P2A, a E2A, or F2A peptide. In an aspect, a GSG linker can be added to the N-terminus of a disclosed 2A peptide. In an aspect, a disclosed encoded P2A peptide can comprise the sequence set forth in SEQ ID NO:15 or SEQ ID NO: 102 or a fragment thereof. In an aspect, a disclosed encoded T2A peptide can comprise the sequence set forth in SEQ ID NO: 103 or SEQ ID NO: 104 or a fragment thereof. In an aspect, a disclosed encoded E2A peptide can comprise the sequence set forth in SEQ ID NO:105 or SEQ ID NO: 106 or a fragment thereof. In an aspect, a disclosed encoded F2A peptide can comprise the sequence set forth in SEQ ID NO: 107 or SEQ ID NO: 108 or a fragment thereof. In an aspect, a disclosed isolated nucleic acid molecule comprising a sequence encoding a disclosed intracellular domain can further comprise a nucleic acid sequence encoding an EGFR domain or a truncated EGFR domain. In an aspect, a disclosed encoded EGFR domain can comprise the sequence set forth in SEQ ID NO:17 or a fragment thereof. In an aspect, a disclosed encoded truncated EGFR domain can comprise the sequence set forth in SEQ ID NO:18 or a fragment thereof. In an aspect, a disclosed isolated nucleic acid molecule comprising a nucleic acid sequence encoding a disclosed intracellular domain can further comprise a nucleic acid sequence encoding a self-cleaving peptide, a signal peptide, and an EGFR domain or truncated EGFR domain, or any combination thereof. In an aspect, a disclosed isolated nucleic acid molecule comprising a nucleic acid sequence encoding a disclosed intracellular domain can further comprise a nucleic acid sequence encoding a disclosed self-cleaving peptide, the signal peptide, and the truncated EGFR domain can comprise the sequence set forth in SEQ ID NO:04 or a fragment thereof. In an aspect, a disclosed isolated nucleic acid molecule encoding a disclosed intracellular domain can further comprise a nucleic acid sequence encoding a disclosed self-cleaving peptide, the signal peptide, and the truncated EGFR domain can comprise a sequence having at least 70%, at least 75%, at least 80%, at least 85%, at least 90%, or at least 95% identity to the sequence set forth in SEQ ID NO:04 or a fragment thereof.

In an aspect, a disclosed isolated nucleic acid molecule can be introduced to T cells and/or NK cells. In an aspect, a disclosed isolated nucleic acid molecule can be used to activated one or more types of immune cells (e.g., naive T cells, central memory T cells, effector memory T cells, NK cells or combination thereof) upon antigen binding.

In an aspect, a disclosed isolated nucleic acid molecule can stimulate an effector cell mediated immune modulator response to PS-expressing tumor cells. In an aspect, a disclosed isolated nucleic acid molecule can induce a tumor reducing immune response. In an aspect, a disclosed isolated nucleic acid molecule can induce phagocytosis of cancer cells in the subject. In an aspect, a disclosed isolated nucleic acid molecule can cross-prime an anti-tumor T cell response. In an aspect, a disclosed isolated nucleic acid molecule can induce a tumor eliminating immune response. In an aspect, a disclosed isolated nucleic acid molecule can treat cancer.

### 3. Vectors

Disclosed herein is a vector comprising a disclosed isolated nucleic acid molecule. Disclosed herein is a vector comprising a disclosed nucleic acid sequence. Disclosed herein a vector comprising an isolated nucleic acid molecule comprising a nucleic acid sequence encoding any disclosed CAR. Disclosed herein a vector comprising an isolated nucleic acid molecule comprising a nucleic acid sequence encoding a CAR specific for phosphatidylserine (PS).

Disclosed herein a vector comprising an isolated nucleic acid molecule comprising a nucleic acid sequence encoding a CAR comprising an extracellular domain comprising an antigen binding domain, a transmembrane domain, and an intracellular domain comprising one or more immunostimulatory domains. Disclosed herein a vector comprising an isolated nucleic acid and an intracellular domain comprising one or more immunoreceptor tyrosine-based activation domains (ITAMs) and/or one or more co-stimulatory domains. Disclosed herein a vector comprising an isolated nucleic acid molecule comprising a nucleic acid sequence encoding a CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof. Disclosed herein a vector comprising an isolated nucleic acid molecule comprising a nucleic acid sequence encoding a phosphatidylserine-specific CAR comprising an extracellular domain comprising an antigen binding domain, a transmembrane domain, and an intracellular domain comprising one or more immunostimulatory domains. Disclosed herein a vector comprising an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein a vector comprising an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof.

Disclosed herein a vector comprising an isolated nucleic acid molecule comprising a nucleic acid sequence encoding a CAR comprising the sequence set forth in SEQ ID NO:01, SEQ ID NO: 02, or SEQ ID NO:03. Disclosed herein a vector comprising an isolated nucleic acid molecule comprising a nucleic acid sequence encoding a CAR comprising the sequence set forth in SEQ ID NO:05, SEQ ID NO:06, or SEQ ID NO:07. Disclosed herein a vector comprising an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:01, SEQ ID NO:02, or SEQ ID NO:03. Disclosed herein a vector comprising an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:05, SEQ ID NO:06, or SEQ ID NO:07.

Disclosed herein a vector comprising an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein a vector comprising an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor specific antigen or fragment thereof.

In an aspect, a disclosed vector can be used to introduce a disclosed isolated nucleic acid molecule or disclosed isolated nucleic acid sequence to one or more host cells. In an aspect, host cells are discussed infra and can comprise T cells, NK cells, macrophages, or iPSCs.

In an aspect, a disclosed vector can be used to introduce a disclosed isolated nucleic acid molecule encoding a disclosed CAR or disclosed isolated nucleic acid sequence encoding a disclosed CAR to one or more host cells.

In an aspect, a disclosed vector can be used to introduce a disclosed isolated nucleic acid molecule or disclosed isolated nucleic acid sequence to one or more T cells or NK cells or macrophages. In an aspect, a disclosed vector can be used to introduce a disclosed isolated nucleic acid molecule encoding a disclosed CAR or disclosed isolated nucleic acid sequence encoding a disclosed CAR to one or more T cells or NK cells or macrophages.

In an aspect, a vector can be an integrating vector or a non-integrating vector. In an aspect, integration can mean that the nucleotides of nucleic acid sequence can be stably inserted into the cellular genome (e.g., covalently linked to the nucleic acid sequence within the cell''s chromosomal DNA).

In an aspect, a disclosed vector can comprise lipid and/or polymer-based nanoparticles loaded with mRNA encoding a disclosed CAR. In an aspect, a disclosed vector can comprise lipid and/or polymer-based nanoparticles loaded with transposon-based plasmids such as, for example, transposon-based plasmids comprising a sequence encoding a disclosed CAR. In an aspect, a disclosed vector can comprise mRNA encoding a disclosed CAR. In an aspect, a disclosed vector can comprise lipid and/or polymer-based nanoparticles loaded with mRNA encoding a disclosed CAR.

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV218, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be a self-complementary AAV as disclosed herein.

In an aspect, a disclosed vector can be a recombinant vector comprising a disclosed nucleic acid sequence. Recombinant vectors (such as recombinant viral vectors) are known to the art.

In an aspect, a disclosed promoter can comprise a ubiquitous promoter, a constitutive promoter, or a tissue specific promoter. In an aspect, a disclosed promoter can be operably linked to a disclosed nucleic acid sequence encoding a disclosed CAR. In an aspect, a disclosed promoter can be operably linked to a disclosed nucleic acid sequence encoding a disclosed tumor antigen. Promoters are known to the art. In an aspect, a disclosed promoter can be a promoter/enhancer. Promoter/enhancers are known to the art. In an aspect, a disclosed promoter can be an endogenous promoter. In an aspect, a disclosed endogenous promoter can be an endogenous promoter/enhancer. In an aspect, a disclosed promoter or a disclosed promoter/enhancer can be used for constitutive and efficient expression of a disclosed CAR. In an aspect, a disclosed promoter or a disclosed promoter/enhancer can be used for constitutive and efficient expression of a disclosed tumor antigen. In an aspect, a disclosed vector can be used to engineer cells to express a disclosed CAR.

In an aspect, a disclosed vector can further comprise a nucleic acid encoding a second CAR that is specific for a tumor antigen. In an aspect, a disclosed tumor antigen comprises can comprise HPV-16 E6 and HPV-16 E7, alpha folate receptor, 5T4, avp6 integrin, BCMA, B7-H3, B7-H6, CAIX, CD19, CD20, CD22, CD28, CD30, CD33, CD44, CD44v6, CD44v7/8, CD70, CD79a, CD79b, CD123, CD137 (4-1BB), CD138, CD171, CEA, CSPG4, CLL-1, CS1, EGFR, EGFR family including ErbB2 (HERII), EGFRvIll, EGP2, EGP40, EPCAM, EphA2, EpCAM, FAP, fetal AchR, FRa, Flt3, GD2, GD3, Glypican-3 (GPC3), HLAA1+MAGE1, HLA-A2+MAGE1, HLAA3+MAGE1, HLA-A1+NY-ES0-1, HLA-A2+NY-ES0-1, HLA-A3+NYES0-1, IL-11Ra, IL-13Ra2, Lambda, Lewis-Y, Kappa, Mesothelin, Mud, Muc16, NCAM, NKG2D Ligands, NYE-SO-1, PRAME, PSCA, PSMA, RORI, SSX, Survivin, TACI, TAG72, TEMs, VEGFRII, or any combination thereof.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, a disclosed vector can be a transposon-based vector such as Sleeping Beauty and PiggyBac, both of which are known in the art. In an aspect, a first plasmid can be loaded with a disclosed nucleic acid sequence encoding a disclosed CAR, named transposon, surrounded by inverted repeats (IRs) that contain short direct repeats (DRs), while a second plasmid encodes the enzyme (transposase) that can recognize the sequences from the first plasmid and cut the transposon out of the first plasmid. Then the disclosed CAR sequence can be successfully delivered into the targeted cell (e.g., a T cell or a NK cell or a macrophage) cytoplasm and inserted randomly into TA dinucleotide base pairs of the recipient DNA sequence. In an aspect, using this vector, stable integration and reliable long-term expression of the disclosed CAR sequence can be achieved.

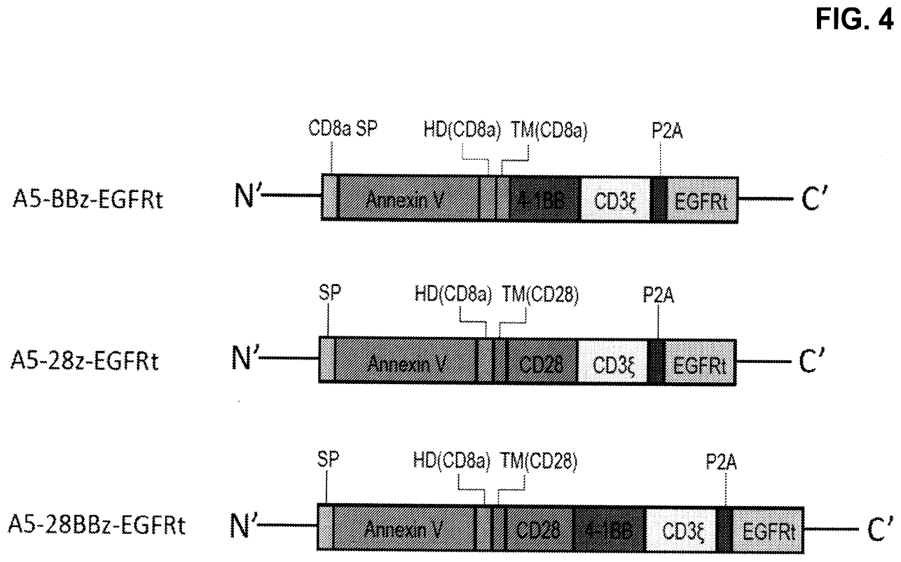

In an aspect, a disclosed vector can stimulate an effector cell-mediated immune modulator response to PS-expressing tumor cells. In an aspect, a disclosed vector can induce a tumor reducing immune response. In an aspect, a disclosed vector can induce phagocytosis of cancer cells in the subject. In an aspect, a disclosed vector can cross-prime an anti-tumor T cell response. In an aspect, a disclosed vector can induce a tumor eliminating immune response. In an aspect, a disclosed vector can treat cancer. Disclosed herein is a vector construct as represented in FIG. 4.

### 4. Plasmids

Disclosed herein is a plasmid comprising a disclosed isolated nucleic acid molecule. Disclosed herein is a vector comprising a disclosed nucleic acid sequence. Disclosed herein a plasmid comprising an isolated nucleic acid molecule comprising a nucleic acid sequence encoding any disclosed CAR. Disclosed herein a plasmid comprising an isolated nucleic acid molecule comprising a nucleic acid sequence encoding a CAR specific for phosphatidylserine (PS). Disclosed herein a plasmid comprising an isolated nucleic acid molecule comprising a nucleic acid sequence encoding a CAR comprising an extracellular domain comprising an antigen binding domain, a transmembrane domain, and an intracellular domain comprising one or more immunostimulatory domains. Disclosed herein a plasmid comprising an isolated nucleic acid and an intracellular domain comprising one or more immunoreceptor tyrosine-based activation domains (ITAMs) and/or one or more co-stimulatory domains. Disclosed herein a plasmid comprising an isolated nucleic acid molecule comprising a nucleic acid sequence encoding a CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof. Disclosed herein a plasmid comprising an isolated nucleic acid molecule comprising a nucleic acid sequence encoding a phosphatidylserine-specific CAR comprising an extracellular domain comprising an antigen binding domain, a transmembrane domain, and an intracellular domain comprising one or more immunostimulatory domains. Disclosed herein a plasmid comprising an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein a plasmid comprising an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof.

Disclosed herein a plasmid comprising an isolated nucleic acid molecule comprising a nucleic acid sequence encoding a CAR comprising the sequence set forth in SEQ ID NO:01, SEQ ID NO: 02, or SEQ ID NO:03. Disclosed herein a plasmid comprising an isolated nucleic acid molecule comprising a nucleic acid sequence encoding a CAR comprising the sequence set forth in SEQ ID NO:05, SEQ ID NO:06, or SEQ ID NO:07. Disclosed herein a plasmid comprising an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:01, SEQ ID NO:02, or SEQ ID NO:03. Disclosed herein a plasmid comprising an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO: 05, SEQ ID NO:06, or SEQ ID NO:07.

Disclosed herein a plasmid comprising an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein a plasmid comprising an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor specific antigen or fragment thereof.

In an aspect, a disclosed plasmid can be used to introduce a disclosed isolated nucleic acid molecule or disclosed isolated nucleic acid sequence to one or more host cells. In an aspect, host cells are discussed infra and can comprise T cells, NK cells, macrophages, or iPSCs.

In an aspect, a disclosed plasmid can be used to introduce a disclosed isolated nucleic acid molecule encoding a disclosed CAR or disclosed isolated nucleic acid sequence encoding a disclosed CAR to one or more host cells.

In an aspect, a a disclosed plasmid can be used to introduce a disclosed isolated nucleic acid molecule or disclosed isolated nucleic acid sequence to one or more T cells or NK cells or macrophages. In an aspect, a disclosed plasmid can be used to introduce a disclosed isolated nucleic acid molecule encoding a disclosed CAR or disclosed isolated nucleic acid sequence encoding a disclosed CAR to one or more T cells or NK cells or macrophages.

In an aspect, a disclosed plasmid can be an integrating vector or a non-integrating vector. In an aspect, integration can mean that the nucleotides of nucleic acid sequence can be stably inserted into the cellular genome (e.g., covalently linked to the nucleic acid sequence within the cell''s chromosomal DNA).

In an aspect, a disclosed plasmid can be a recombinant vector comprising a disclosed nucleic acid sequence. Recombinant plasmids are known to the art.

In an aspect, a disclosed promoter can comprise a ubiquitous promoter, a constitutive promoter, or a tissue specific promoter. In an aspect, a disclosed promoter can be operably linked to a disclosed nucleic acid sequence encoding a disclosed CAR. In an aspect, a disclosed promoter can be operably linked to a disclosed nucleic acid sequence encoding a disclosed tumor antigen. Promoters are known to the art. In an aspect, a disclosed promoter can be a promoter/enhancer. Promoter/enhancers are known to the art. In an aspect, a disclosed promoter can be an endogenous promoter. In an aspect, a disclosed endogenous promoter can be an endogenous promoter/enhancer. In an aspect, a disclosed promoter or a disclosed promoter/enhancer can be used for constitutive and efficient expression of a disclosed CAR. In an aspect, a disclosed promoter or a disclosed promoter/enhancer can be used for constitutive and efficient expression of a disclosed tumor antigen. In an aspect, a disclosed plasmid can be used to engineer cells to express a disclosed CAR.

In an aspect, a disclosed plasmid can further comprise a nucleic acid encoding a second CAR that is specific for a tumor antigen. In an aspect, a disclosed tumor antigen comprises can comprise HPV-16 E6 and HPV-16 E7, alpha folate receptor, 5T4, avp6 integrin, BCMA, B7-H3, B7-H6, CAIX, CD19, CD20, CD22, CD28, CD30, CD33, CD44, CD44v6, CD44v7/8, CD70, CD79a, CD79b, CD123, CD137 (4-1BB), CD138, CD171, CEA, CSPG4, CLL-1, CS1, EGFR, EGFR family including ErbB2 (HERII), EGFRvIll, EGP2, EGP40, EPCAM, EphA2, EpCAM, FAP, fetal AchR, FRa, Flt3, GD2, GD3, Glypican-3 (GPC3), HLAA1+MAGE1, HLA-A2+MAGE1, HLAA3+MAGE1, HLA-A1+NY-ES0-1, HLA-A2+NY-ES0-1, HLA-A3+NYES0-1, IL-11Ra, IL-13Ra2, Lambda, Lewis-Y, Kappa, Mesothelin, Mud, Muc16, NCAM, NKG2D Ligands, NYE-SO-1, PRAME, PSCA, PSMA, RORI, SSX, Survivin, TACI, TAG72, TEMs, VEGFRII, or any combination thereof.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, a disclosed plasmid can comprise a transposons such as, for example, Sleeping Beauty and PiggyBac, both of which are known in the art. In an aspect, a first plasmid can be loaded with a disclosed nucleic acid sequence encoding a disclosed CAR, named transposon, surrounded by inverted repeats (IRs) that contain short direct repeats (DRs), while a second plasmid encodes the enzyme (transposase) that can recognize the sequences from the first plasmid and cut the transposon out of the first plasmid. Then the disclosed CAR sequence can be successfully delivered into the targeted cell (e.g., a T cell or a NK cell or a macrophage) cytoplasm and inserted randomly into TA dinucleotide base pairs of the recipient DNA sequence. In an aspect, using this vector, stable integration and reliable long-term expression of the disclosed CAR sequence can be achieved.

In an aspect, a disclosed plasmid can stimulate an effector cell mediated immune modulator response to PS-expressing tumor cells. In an aspect, a disclosed plasmid can induce a tumor reducing immune response. In an aspect, a disclosed plasmid can induce phagocytosis of cancer cells in the subject. In an aspect, a disclosed plasmid can cross-prime an anti-tumor T cell response. In an aspect, a disclosed plasmid can induce a tumor eliminating immune response. In an aspect, a disclosed plasmid can treat cancer.

### 5. Cells

Disclosed herein are cells transformed or transfected by one or more disclosed isolated nucleic acid molecules. Disclosed herein are cells transformed or transfected by an isolated nucleic acid molecule comprising a nucleic acid sequence encoding any disclosed CAR. Disclosed herein are cells transformed or transfected by an isolated nucleic acid molecule comprising a nucleic acid sequence encoding a CAR specific for phosphatidylserine (PS).

Disclosed herein are cells transformed or transfected by an isolated nucleic acid molecule comprising a nucleic acid sequence encoding a CAR comprising an extracellular domain comprising an antigen binding domain, a transmembrane domain, and an intracellular domain comprising one or more immunostimulatory domains. Disclosed herein are cells transformed or transfected by an isolated nucleic acid molecule comprising a nucleic acid sequence encoding a CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein are cells transformed or transfected by an isolated nucleic acid molecule comprising a nucleic acid sequence encoding a CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof. Disclosed herein are cells transformed or transfected by an isolated nucleic acid molecule comprising a nucleic acid sequence encoding a phosphatidylserine-specific CAR comprising an extracellular domain comprising an antigen binding domain, a transmembrane domain, and an intracellular domain comprising one or more immunostimulatory domains.

Disclosed herein are cells transformed or transfected by an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein are cells transformed or transfected by an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof.

Disclosed herein are cells transformed or transfected by an isolated nucleic acid molecule comprising a nucleic acid sequence encoding a CAR comprising the sequence set forth in SEQ ID NO: 01, SEQ ID NO:02, or SEQ ID NO:03. Disclosed herein are cells transformed or transfected by an isolated nucleic acid molecule comprising a nucleic acid sequence encoding a CAR comprising the sequence set forth in SEQ ID NO:05, SEQ ID NO:06, or SEQ ID NO:07.

Disclosed herein are cells transformed or transfected by an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:01, SEQ ID NO:02, or SEQ ID NO:03. Disclosed herein are cells transformed or transfected by an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:05, SEQ ID NO:06, or SEQ ID NO:07.

Disclosed herein are cells transformed or transfected by an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains.

Disclosed herein are cells transformed or transfected by an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor specific antigen or fragment thereof. Disclosed herein are cells transduced by one or more disclosed viral vectors.

Disclosed herein are cells transduced by a vector comprising a disclosed isolated nucleic acid molecule. Disclosed herein are cells transduced by a vector comprising a disclosed nucleic acid sequence. Disclosed herein are cells transduced by a vector comprising an isolated nucleic acid molecule comprising a nucleic acid sequence encoding any disclosed CAR. Disclosed herein are cells transduced by a vector comprising an isolated nucleic acid molecule comprising a nucleic acid sequence encoding a CAR specific for phosphatidylserine (PS).

Disclosed herein are cells transduced by a vector comprising an isolated nucleic acid molecule comprising a nucleic acid sequence encoding a CAR comprising an extracellular domain comprising an antigen binding domain, a transmembrane domain, and an intracellular domain comprising one or more immunostimulatory domains. Disclosed herein are cells transduced by a vector comprising an isolated nucleic acid molecule comprising a nucleic acid sequence encoding a CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein are cells transduced by a vector comprising an isolated nucleic acid molecule comprising a nucleic acid sequence encoding a CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof. Disclosed herein are cells transduced by a vector comprising an isolated nucleic acid molecule comprising a nucleic acid sequence encoding a phosphatidylserine-specific CAR comprising an extracellular domain comprising an antigen binding domain, a transmembrane domain, and an intracellular domain comprising one or more immunostimulatory domains. Disclosed herein are cells transduced by a vector comprising an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein are cells transduced by a vector comprising an isolated nucleic acid molecule comprising a nucleic acid sequence encoding a phosphatidylserine-specific CAR comprising from N-terminus to C-terminus a signal peptide; an antigen binding domain; a hinge domain; a transmembrane domain; an intracellular domain, a cleavage signal; a signal peptide; and a tumor specific antigen or fragment thereof.

Disclosed herein are cells transduced by a vector comprising an isolated nucleic acid molecule comprising a nucleic acid sequence encoding a CAR comprising the sequence set forth in SEQ ID NO:01, SEQ ID NO:02, or SEQ ID NO:03. Disclosed herein are cells transduced by a vector comprising an isolated nucleic acid molecule comprising a nucleic acid sequence encoding a CAR comprising the sequence set forth in SEQ ID NO:05, SEQ ID NO:06, or SEQ ID NO: 07. Disclosed herein are cells transduced by a vector comprising an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO:01, SEQ ID NO:02, or SEQ ID NO:03. Disclosed herein are cells transduced by a vector comprising an isolated nucleic acid molecule comprising a nucleic acid sequence encoding a CAR comprising a sequence having at least 70%, at least 75%, at least 80%, at least 85%, at least 90%, or at least 95% identity to the sequence set forth in SEQ ID NO: 05, SEQ ID NO:06, or SEQ ID NO:07.

Disclosed herein are cells transduced by a vector comprising an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; and an intracellular domain comprising one or more immunoreceptor tyrosine-based activation domains (ITAMs) and/or one or more co-stimulatory domains. Disclosed herein are cells transduced by a vector comprising an isolated nucleic acid molecule comprising a nucleic acid sequence encoding a chimeric antigen receptor (CAR), comprising from N-terminus to C-terminus a signal peptide; an antigen binding domain; a hinge domain; a transmembrane domain; an intracellular domain, a cleavage signal; a signal peptide; and a tumor specific antigen or fragment thereof.

Disclosed herein is a cell expressing an anti-phosphatidylserine (PS) chimeric antigen receptor (CAR), wherein anti-PS CAR comprises a disclosed CAR. Disclosed herein is a cell expressing a chimeric antigen receptor (CAR), wherein the CAR comprises an anti-PS binding domain, wherein the bind domain comprises Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect, a disclosed cell can be transformed or transfected by an isolated nucleic acid molecule comprising a nucleic acid sequence encoding a tumor antigen. In an aspect, a disclosed cell can be transduced by a vector comprising an isolated nucleic acid molecule comprising a nucleic acid sequence encoding a tumor antigen. In an aspect, following transformation, transfection, and/or transduction, a disclosed cell can express a disclosed CAR and/or a disclosed tumor antigen.

In an aspect, disclosed cells can comprise T cells or NK cells or macrophages. In an aspect, disclosed cells are immune cells. In an aspect, disclosed cells can comprise T cells, B cells, natural killer (NK) cells, dendritic cells, granulocytes, innate lymphoid cells, megakaryocytes, monocytes, macrophages, platelets, thymocytes, myeloid cells, or any combination thereof. In an aspect, disclosed T cells and NK cells can be differentiated in vitro from a hematopoietic stem cell population (for example iPSCs) or can be obtained from a subject. In an aspect, T cells and NK cells can be obtained from, for example, peripheral blood mononuclear cells (PBMCs), bone marrow, lymph node tissue, cord blood, thymus tissue, tissue from a site of infection, ascites, pleural effusion, spleen tissue, tumors, or any combination thereof. In an aspect, disclosed T cells can be derived from one or more T cell lines available in the art. In an aspect, disclosed T cells can also be obtained from a unit of blood collected from a subject using any number of techniques known to the skilled person.

In an aspect, following transformation, transfection, and/or transduction, disclosed host cells expressing a disclosed CAR and/or a disclosed tumor antigen can be administered to a subject. In an aspect, the transformed, transfected, and/or transduced, disclosed host cells expressing a disclosed CAR and/or a disclosed tumor antigen can be autologous to the receiving subject.

In an aspect, a disclosed cell can stimulate an effector cell-mediated immune modulator response to PS-expressing tumor cells. In an aspect, a disclosed cell can induce a tumor-reducing immune response. In an aspect, a disclosed cell can induce phagocytosis of cancer cells in the subject. In an aspect, a disclosed cell can cross-prime an anti-tumor T cell response. In an aspect, a disclosed cell can induce a tumor-eliminating immune response. In an aspect, a disclosed cell can treat cancer.

In an aspect, a therapeutically effective amount of the disclosed genetically modified cells expressing a disclosed chimeric antigen receptor targeting phosphatidylserine (PS) (e.g., T cells, NK cells, and/or macrophages) can be about 1×10⁴ to about 1×10⁹ cells/kg per subject. In an aspect, a therapeutically effective amount of the disclosed genetically modified cells expressing a disclosed chimeric antigen receptor targeting phosphatidylserine (PS) (e.g., T cells, NK cells, and/or macrophages) can be about 1×10⁵ cells/kg per subject, about 1×10⁶ cells/kg per subject, about 1×10⁷ cells/kg per subject, about 1×10⁸ cells/kg per subject, or about 1×10⁹ cells/kg per subject.

### 6. Pharmaceutical Formulations

Disclosed herein is a pharmaceutical formulation comprising a disclosed isolated nucleic acid molecule; and one or more pharmaceutically acceptable carriers. Disclosed herein is a pharmaceutical formulation comprising a disclosed vector; and one or more pharmaceutically acceptable carriers. Disclosed herein is a pharmaceutical formulation comprising a disclosed plasmid; and one or more pharmaceutically acceptable carriers. Disclosed herein is a pharmaceutical formulation comprising a disclosed cell; and one or more pharmaceutically acceptable carriers.

Disclosed herein is a pharmaceutical formulation comprising an isolated nucleic acid molecule comprising a nucleic acid sequence encoding any disclosed CAR. Disclosed herein is an isolated nucleic acid molecule comprising a nucleic acid sequence encoding a CAR specific for phosphatidylserine (PS). Disclosed herein is a pharmaceutical formulation an isolated nucleic acid molecule comprising a nucleic acid sequence encoding a CAR comprising an extracellular domain comprising an antigen binding domain, a transmembrane domain, and an intracellular domain comprising one or more immunostimulatory domains.

Disclosed herein is a pharmaceutical formulation comprising a vector comprising a disclosed nucleic acid sequence. Disclosed herein is a pharmaceutical formulation comprising a vector comprising an isolated nucleic acid molecule comprising a nucleic acid sequence encoding any disclosed CAR. Disclosed herein a vector comprising an isolated nucleic acid molecule comprising a nucleic acid sequence encoding a CAR specific for phosphatidylserine (PS).

Disclosed herein is a pharmaceutical formulation comprising a plasmid comprising a disclosed isolated nucleic acid molecule. Disclosed herein is a pharmaceutical formulation comprising a plasmid comprising a disclosed nucleic acid sequence. Disclosed herein is a pharmaceutical formulation comprising a plasmid comprising an isolated nucleic acid molecule comprising a nucleic acid sequence encoding any disclosed CAR. Disclosed herein is a pharmaceutical formulation comprising a plasmid comprising an isolated nucleic acid molecule comprising a nucleic acid sequence encoding a CAR specific for phosphatidylserine (PS). Disclosed herein is a pharmaceutical formulation comprising a plasmid comprising an isolated nucleic acid molecule comprising a nucleic acid sequence encoding a CAR comprising an extracellular domain comprising an antigen binding domain, a transmembrane domain, and an intracellular domain comprising one or more immunostimulatory domains.

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof.

In an aspect, a disclosed pharmaceutical formulation can further comprise one or more anti-inflammatory agents. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

In an aspect, NSAIDs can comprise ibuprofen, naproxen, naproxen sodium, Cox-2 inhibitors such as rofecoxib and celecoxib, sialylates, or any combination thereof. In an aspect, analgesics can comprise acetaminophen, oxycodone, tramadol, proporxyphene hydrochloride, or any combination thereof. In an aspect, glucocorticoids can comprise cortisone, dexamethasone, hydrocortisone, methylprednisolone, prednisolone, prednisone, or any combination thereof. Exemplary biological response modifiers include molecules directed against cell surface markers (e.g., CD4, CD5, etc.), cytokine inhibitors, such as the TNF antagonists (e.g., etanercept, adalimumab, and infliximab, chemokine inhibitors and adhesion molecule inhibitors. In an aspect, biological response modifiers can comprise monoclonal antibodies as well as recombinant forms of molecules. In an aspect, exemplary disease-modifying anti-rheumatic drugs (DMARDs) can comprise include azathioprine, cyclophosphamide, cyclosporine, methotrexate, penicillamine, leflunomide, sulfasalazine, hydroxychloroquine, Gold (oral (auranofin) and intramuscular), minocycline, or any combination thereof.

In an aspect, a disclosed chemotherapeutic agent in a disclosed pharmaceutical formulation can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed pharmaceutical formulation can comprise an anti-chemokine therapy that enhances the resident memory T cell formations in tumor-free tissues. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a disclosed pharmaceutical formulation can further comprise abagovomab, adecatumumab, afutuzumab, alemtuzumab, altumomab, amatuximab, anatumomab, arcitumomabm bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, or any combination thereof.

In an aspect, a disclosed pharmaceutical formulation can stimulate an effector cell mediated immune response to PS-expressing tumor cells. In an aspect, a disclosed pharmaceutical formulation can restore one or more aspects of cellular homeostasis and/or cellular functionality and/or metabolic dysregulation in a subject having cancer. In an aspect, metabolic dysregulation can be associated with cancer or cancerous cells. In an aspect, following administration of a disclosed pharmaceutical formulation, cell death of PS-expressing cancer cells is effected.

In an aspect, a disclosed pharmaceutical formulation can be prepared for systemic or direct administration. In an aspect, a disclosed pharmaceutical formulation can be prepared for oral administration, intravenous administration, intratumoral administration, intraperitoneal administration, or any combination thereof. In an aspect, a disclosed pharmaceutical formulation can be prepared for any method of administration disclosed herein. In an aspect, a disclosed pharmaceutical formulation can be prepared for administration via multiple routes either concurrently or sequentially. For example, in an aspect, a disclosed pharmaceutical formulation can be first administered intratumorally and then be administered intravenously. In an aspect, a disclosed pharmaceutical formulation can be first administered intratumorally and then be administered orally. A skilled clinical can determine the best route of administration for a subject at a given time.

In an aspect, a disclosed pharmaceutical formulation can comprise one or more immune modulators. In an aspect, a disclosed pharmaceutical formulation can comprise one or more proteasome inhibitors. In an aspect, a disclosed pharmaceutical formulation can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed pharmaceutical formulation can comprise an anaplerotic agent (such as, for example, C7 compounds like triheptanoin or MCT).

In an aspect, a disclosed pharmaceutical formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme (such as, for example, a missing, deficient, and/or mutant protein or enzyme related to cancer and/or associated with cancerous cells). In an aspect, a disclosed RNA therapeutic can be directed at any protein or enzyme that is overexpressed or is overactive and related to cancer and/or associated with cancerous cells.

In an aspect, a disclosed pharmaceutically acceptable carrier can comprise any disclosed carrier and/or any disclosed excipient.

In an aspect, a disclosed pharmaceutical formulation can stimulate an effector cell mediated immune modulator response to PS-expressing tumor cells. In an aspect, a disclosed pharmaceutical formulation can induce a tumor reducing immune response. In an aspect, a disclosed pharmaceutical formulation can induce phagocytosis of cancer cells in the subject. In an aspect, a disclosed pharmaceutical formulation can cross-prime an anti-tumor T cell response. In an aspect a disclosed pharmaceutical formulation can induce a tumor eliminating immune response. In an aspect, a disclosed pharmaceutical formulation can treat cancer.

### 7. Animals

Disclosed herein are animals used to validate the efficacy and/or safety of one or more disclosed CARs, one or more disclosed cells, one or more disclosed isolated nucleic acid molecules, one or more disclosed vectors, one or more disclosed pharmaceutical formulations, or any combination thereof. In an aspect, a disclosed animal can be treated with one or more disclosed CARs, one or more disclosed cells, one or more disclosed isolated nucleic acid molecules, one or more disclosed vectors, one or more disclosed pharmaceutical formulations, or any combination thereof. In an aspect, animals can be assessed and/or monitored for one or more biological and/or chemical functions prior to treatment, during treatment, after treatment, or any combination thereof. In an aspect, a disclosed treated subject can be a mouse or a rat. In an aspect, a disclosed treated subject can be a transgenic mouse or a transgenic rat. In an aspect, a disclosed treated subject can have one or more types of cancers and/or tumors.

### E. Methods of Treating Cancer

Disclosed herein is a method of treating cancer, the method comprising treating a subject in need thereof by administering to the subject in need thereof a therapeutically effective amount of one or more cells transduced with a disclosed recombinant vector or a pharmaceutical formulation thereof, wherein the one or more cells express a CAR. Disclosed herein is a method of treating cancer, the method comprising treating a subject in need thereof by administering to the subject in need thereof a therapeutically effective amount of one or more cells transformed with a disclosed plasmid or a pharmaceutical formulation thereof, wherein the one or more cells express a CAR. Disclosed herein is a method of treating cancer, the method comprising treating a subject in need thereof by administering to the subject in need thereof a therapeutically effective amount of one or more genetically modified cells expressing a chimeric antigen receptor targeting phosphatidylserine (PS) or a pharmaceutical formulation thereof. Disclosed herein is a method of treating cancer, the method comprising treating a subject in need thereof by administering to the subject in need thereof a therapeutically effective amount of one or more immune cells transduced with a recombinant vector or a pharmaceutical formulation thereof, wherein the one or more cells express a CAR. Disclosed herein is a method of treating cancer, the method comprising treating a subject in need thereof by administering to the subject in need thereof a therapeutically effective amount of one or more immune cells transformed with a disclosed plasmid or a pharmaceutical formulation thereof, wherein the one or more cells express a CAR. Disclosed herein is a method of treating cancer, the method comprising treating a subject in need thereof by administering to the subject in need thereof a therapeutically effective amount of one or more cells expressing a chimeric antigen receptor targeting phosphatidylserine (PS) or a pharmaceutical formulation thereof. Disclosed herein is a method of treating cancer, the method comprising treating a subject in need thereof by administering to the subject in need thereof a therapeutically effective amount of one or more cells expressing a chimeric antigen receptor targeting phosphatidylserine-expressing tumor cells or a pharmaceutical formulation thereof.

In an aspect, wherein the CAR targets phosphatidylserine (PS)-expressing cancer cells.

In an aspect, disclosed PS-expressing cancer cells can be in a tumor. In an aspect, disclosed PS-expressing cancer cells can be in one or more tumors. In an aspect, disclosed cancer cells can be blood borne.

In an aspect, a disclosed method of treating cancer can comprise stimulating an effector cell mediated immune modulator response to PS-expressing tumor cells. In an aspect, a disclosed method of treating cancer can induce a tumor reducing immune response. In an aspect, a disclosed method of treating cancer can induce phagocytosis of cancer cells in the subject. In an aspect, a disclosed method of treating cancer can cross-primer an anti-tumor T cell response. In an aspect, a disclosed method of treating cancer can induce a tumor eliminating immune response. In an aspect, a disclosed method of treating cancer can effect tumor cell death.

In an aspect, a disclosed method of treating cancer can comprise transducing extracted T cells or NK cells or macrophages ex vivo with a disclosed vector or a disclosed nucleic acid molecule such that the T cells, NK cells, or macrophages express a disclosed CAR, and returning the CAR-expressing T cells, NK cells, or macrophages back to the subject.

In an aspect, the cells can be autologous to the subject.

In an aspect, cancer cells can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method of treating cancer can further comprise collecting one or more blood and/or biological samples from a subject at the same time or at different times. For example, in an aspect, a blood sample and/or a biological sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample and/or a biological sample can be collected from a subject prior to treatment, during treatment, after treatment, or any combination thereof. In an aspect, a blood and/or a biological sample can be collected from a subject at any time deemed medically and/or clinically appropriate by the skilled clinician.

In an aspect, a disclosed method of treating cancer can further comprise isolating monocytes from peripheral blood monocular cells in the subject''s blood and/or biological sample. In an aspect, a disclosed method of treating cancer can further comprise isolating bone marrow derived monocytes from the subject''s blood and/or biological sample. In an aspect, a disclosed method of treating cancer can further comprise isolating monocytes from the subject''s blood and/or biological sample. In an aspect, a disclosed method of treating cancer can further comprise isolating naïve macrophages (M0) from the subject''s blood and/or biological sample. In an aspect, a disclosed method of treating cancer can further comprise subjecting a disclosed blood sample to centrifugation. In an aspect, a disclosed method can further comprise separating the blood and/or biological sample into its component parts using, for example, centrifugation, apheresis, or any technique to the skilled person. In an aspect, a disclosed separating step can comprise generating a layer of clear fluid, a layer of red fluid, and a thin layer in between the clear fluid layer and the red fluid layer. In an aspect, a disclosed red layer can comprise red blood cells. In an aspect, a disclosed clear layer can comprise plasma. In an aspect, a disclosed thin layer in between the red layer and the clear layer can comprise the buffy coat. In an aspect, a disclosed buffy coat can comprise white blood cells and platelets. In an aspect, a disclosed method can further comprise isolating peripheral blood mononuclear cells (PMBCs) from the buffy coat. In an aspect, PMBCs can comprise lymphocytes, leukocytes, and/or monocytes. In an aspect, macrophages can be derived from monocytes. In an aspect, isolating lymphocytes, leukocytes, and/or monocytes can be done by any method and/or technique known to the skilled person (e.g., leukapheresis).

In an aspect, a disclosed method of treating cancer can further comprise isolating resting or M0 macrophages from the buffy coat. In an aspect, a disclosed method of treating cancer can further comprise differentiating monocytes into resting or M0 macrophages. In an aspect of a disclosed method of treating cancer, the disclosed macrophages can be resting or M0 macrophages. In an aspect, a disclosed method of treating cancer can further comprise polarizing the resting or M0 macrophages into a M1 phenotype or a M2 phenotype or a pro-inflammatory phenotype or an anti-inflammatory phenotype. In an aspect, a disclosed method of treating cancer can further comprise polarizing the resting or M0 macrophages into a classically activated macrophage phenotype.

In an aspect, a therapeutically effective amount or effective dose or effective amount or therapeutically effective dosage of disclosed engineered T cells or NK cells or macrophages can be any amount that, when used alone or in combination with another therapeutic agent, can attack and destroy PS-expressing tumor cells. In an aspect, a therapeutically effective amount or effective dose or effective amount or therapeutically effective dosage of the disclosed genetically modified cells expressing a chimeric antigen receptor targeting phosphatidylserine (PS) or a pharmaceutical formulation thereof or the disclosed engineered T cells or NK cells or macrophages expressing a chimeric antigen receptor targeting phosphatidylserine (PS) or a pharmaceutical formulation thereof can be any amount that, when used alone or in combination with another therapeutic agent, can attack and destroy PS-expressing tumor cells.

In an aspect, a therapeutically effective amount of the disclosed genetically modified cells expressing a disclosed chimeric antigen receptor targeting phosphatidylserine (PS) (e.g., T cells, NK cells, and/or macrophages) can be about 1×10⁴ to about 1×10⁹ cells/kg per subject. In an aspect, a therapeutically effective amount of the disclosed genetically modified cells expressing a disclosed chimeric antigen receptor targeting phosphatidylserine (PS) (e.g., T cells, NK cells, and/or macrophages) can be about 1×10⁵ cells/kg per subject, about 1×10⁶ cells/kg per subject, about 1×10⁷ cells/kg per subject, about 1×10⁸ cells/kg per subject, or about 1×10⁹ cells/kg per subject.

In an aspect, a therapeutically effective amount or effective dose or effective amount or therapeutically effective dosage of the disclosed genetically modified cells expressing a chimeric antigen receptor targeting phosphatidylserine (PS) or a pharmaceutical formulation thereof or the disclosed engineered T cells or NK cells or macrophages expressing a chimeric antigen receptor targeting phosphatidylserine (PS) or a pharmaceutical formulation thereof can protect a subject against the onset of a disease and/or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In an aspect, administering genetically modified cells expressing a chimeric antigen receptor targeting phosphatidylserine (PS) or a pharmaceutical formulation thereof or administering engineered T cells or NK cells or macrophages expressing a chimeric antigen receptor targeting phosphatidylserine (PS) or a pharmaceutical formulation thereof can comprise systemic or direct administration. In an aspect, administering can comprise oral administration, intravenous administration, intratumoral administration, intraperitoneal administration, or any combination thereof. In an aspect, administering genetically modified cells expressing a chimeric antigen receptor targeting phosphatidylserine (PS) or a pharmaceutical formulation thereof or administering engineered T cells or NK cells or macrophages expressing a chimeric antigen receptor targeting phosphatidylserine (PS) or a pharmaceutical formulation thereof can be administered by any method of administration disclosed herein. In an aspect, genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof can be administered via multiple routes either concurrently or sequentially.

For example, in an aspect, genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof can be first administered intratumorally and then be administered intravenously. In an aspect, genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof can be first administered intratumorally and then be administered orally. A skilled clinician can determine the best route of administration for a subject at a given time.

In an aspect, a disclosed method can comprise repeating the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof.

In an aspect, a disclosed method of treating cancer can comprise protecting the subject from metastasis. In an aspect, a disclosed method of treating cancer can comprise reducing the risk of developing metastasis. In an aspect, a disclosed method of treating cancer can comprise preventing or inhibiting metastasis.

In an aspect, a disclosed method can comprise monitoring the subject for adverse effects. In an aspect, in the absence of adverse effects, a disclosed method can comprise continuing to treat the subject. In an aspect, continuing to treat the subject can comprise continuing to administer the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof administered to the subject, changing the frequency of administration of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof, changing the duration of administration of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof, changing the route of administration of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof, or any combination thereof.

In an aspect, a disclosed method of treating cancer can further comprise administering to the subject an immune checkpoint inhibitor (e.g., an anti-PD1 molecule). In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed method of treating cancer can further comprise repeating the administering of the anti-PD1 molecule. In an aspect, a disclosed anti-PD1 molecule can be administered prior to, concurrent with, or after the administration of the interfering molecule.

In an aspect of a disclosed method of treating cancer, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

For example, in an aspect, a disclosed anti-PD1 molecule can be administered about 3 months, about 2 months, or about 1 month prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof.

In an aspect, a disclosed method of treating cancer can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method of treating cancer can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a disclosed method of treating cancer can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of treating cancer can further comprise preventing or inhibiting metastasis of cancer cells. In an aspect, a disclosed method can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof)). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof).

In an aspect, a disclosed method of treating cancer can comprise surgically resecting the tumor and/or cancer cells from the subject. In an aspect, following resecting the tumor and/or cancer cells from the subject, a disclosed method of treating cancer can comprise continuing to administer to the subject a therapeutically effective amount of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof and continuing to administer to the subject a therapeutically effective amount of an anti-PD1 molecule, a disclosed anti-chemokine therapy, a disclosed chemotherapeutic agent, any disclosed therapeutic agent, or any combination thereof.

In an aspect, a disclosed method of treating cancer can further comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of treating cancer can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed method of treating cancer, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid Body (Carotid |
| Volume/Na⁺ Conc./ | Artery), Atrial Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs (Hypothalamus) |
| pO₂, pCO₂, and pH | Aortic Body (Aorta), Carotid Body (Carotid |
| | Artery), Ventrolateral Medulla (Medulla) |
| Temperature | Thermosensory neurons (Skin), Preoptic Area |
| | (Hypothalamus) |

In an aspect, a disclosed method of treating cancer can further comprise administering genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof one or more times, administering an anti-PD1 molecule one or more times, administering one or more anti-cancer therapies one or more times, or administering any combination thereof one or more time.

In an aspect, preventing or inhibiting metastasis of cancer cells can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof).

In an aspect, a disclosed method of treating cancer can comprise surgically resecting the tumor and/or cancer cells from the subject. In an aspect, following resecting the tumor and/or cancer cells from the subject, a disclosed method of treating cancer can comprise continuing to administer to the subject a therapeutically effective amount of a disclosed pharmaceutical formulation. In an aspect, a disclosed method of treating cancer can further comprise diagnosing the subject as have cancer or cancerous cells.

In an aspect, a disclosed method of treating cancer can improve and/or extend the survivability of the subject, can improve a subject''s quality of life, can increase and/or prolong a subject''s life span, or any combination thereof. In an aspect, a disclosed method of treating cancer can stimulate an effector cell mediated immune modulator response to PS-expressing tumor cells. In an aspect, a disclosed method of treating cancer can induce a tumor reducing immune response. In an aspect, a disclosed method of treating cancer can induce phagocytosis of cancer cells in the subject. In an aspect, a disclosed method of treating cancer can cross-prime an anti-tumor T cell response. In an aspect, a disclosed method of treating cancer can induce a tumor eliminating immune response. In an aspect, a disclosed pharmaceutical formulation can treat cancer.

In an aspect, a disclosed method of treating cancer can comprise administering to the subject a chimeric fusion receptor comprising a PS-binding domain.

### F. Methods of Stimulating an Effector Cell Mediated Immune Modulator Response

Disclosed herein is a method of stimulating an effector cell mediated immune modulator response to PS-expressing tumor cells, the method comprising administering to a subject in need thereof a therapeutically effective amount of one or more cells transduced with a disclosed recombinant vector or a pharmaceutical formulation thereof, wherein the one or more cells express a CAR. Disclosed herein is a method of stimulating an effector cell mediated immune modulator response to PS-expressing tumor cells, the method comprising administering to a subject in need thereof therapeutically effective amount of one or more cells transformed with a disclosed plasmid or a pharmaceutical formulation thereof, wherein the one or more cells express a CAR. Disclosed herein is a method of stimulating an effector cell mediated immune modulator response to PS-expressing tumor cells, the method comprising administering to a subject in need thereof a therapeutically effective amount of one or more genetically modified cells expressing a chimeric antigen receptor targeting phosphatidylserine (PS) or a pharmaceutical formulation thereof. Disclosed herein is a method of stimulating an effector cell mediated immune modulator response to PS-expressing tumor cells, the method comprising administering to a subject in need thereof a therapeutically effective amount of one or more immune cells transduced with a recombinant vector or a pharmaceutical formulation thereof, wherein the one or more cells express a CAR.

Disclosed herein is a method of stimulating an effector cell mediated immune modulator response to PS-expressing tumor cells, the method comprising administering to a subject in need thereof a therapeutically effective amount of one or more immune cells transformed with a disclosed plasmid or a pharmaceutical formulation thereof, wherein the one or more cells express a CAR. Disclosed herein is a method of stimulating an effector cell mediated immune modulator response to PS-expressing tumor cells, the method comprising administering to a subject in need thereof a therapeutically effective amount of one or more cells expressing a chimeric antigen receptor targeting phosphatidylserine (PS) or a pharmaceutical formulation thereof. Disclosed herein is a method of stimulating an effector cell mediated immune modulator response to PS-expressing tumor cells, the method comprising administering to a subject in need thereof a therapeutically effective amount of one or more cells expressing a chimeric antigen receptor targeting phosphatidylserine-expressing tumor cells or a pharmaceutical formulation thereof.

In an aspect, wherein the CAR targets phosphatidylserine (PS)-expressing cancer cells.

In an aspect, disclosed PS-expressing cancer cells can be in a tumor. In an aspect, disclosed PS-expressing cancer cells can be in one or more tumors. In an aspect, disclosed cancer cells can be blood borne.

In an aspect, a disclosed method of stimulating an effector cell mediated immune modulator response to PS-expressing tumor cell can treat cancer. In an aspect, a disclosed method of stimulating an effector cell mediated immune modulator response to PS-expressing tumor cells can induce a tumor reducing immune response. In an aspect, a disclosed method of stimulating an effector cell mediated immune modulator response to PS-expressing tumor cells can induce phagocytosis of cancer cells in the subject. In an aspect, a disclosed method of stimulating an effector cell mediated immune modulator response to PS-expressing tumor cells can cross-primer an anti-tumor T cell response. In an aspect, a disclosed method of stimulating an effector cell mediated immune modulator response to PS-expressing tumor cells cancer can induce a tumor eliminating immune response.

In an aspect, a disclosed method of stimulating an effector cell mediated immune modulator response to PS-expressing tumor cells can comprise transducing extracted T cells or NK cells or macrophages ex vivo with a disclosed vector or a disclosed nucleic acid molecule such that the T cells, NK cells, or macrophages express a disclosed CAR, and returning the CAR-expressing T cells, NK cells, or macrophages back to the subject.

In an aspect, the cells can be autologous to the subject.

In an aspect, cancer cells can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method of stimulating an effector cell mediated immune modulator response to PS-expressing tumor cells can further comprise collecting one or more blood and/or biological samples from a subject at the same time or at different times. For example, in an aspect, a blood sample and/or a biological sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample and/or a biological sample can be collected from a subject prior to treatment, during treatment, after treatment, or any combination thereof. In an aspect, a blood and/or a biological sample can be collected from a subject at any time deemed medically and/or clinically appropriate by the skilled clinician.

In an aspect, a disclosed method of stimulating an effector cell mediated immune modulator response to PS-expressing tumor cells can further comprise isolating monocytes from peripheral blood monocular cells in the subject''s blood and/or biological sample. In an aspect, a disclosed method of stimulating an effector cell mediated immune modulator response to PS-expressing tumor cells can further comprise isolating bone marrow derived monocytes from the subject''s blood and/or biological sample. In an aspect, a disclosed method of stimulating an effector cell mediated immune modulator response to PS-expressing tumor cells can further comprise isolating monocytes from the subject''s blood and/or biological sample. In an aspect, a disclosed method of stimulating an effector cell mediated immune modulator response to PS-expressing tumor cells can further comprise isolating naïve macrophages (M0) from the subject''s blood and/or biological sample. In an aspect, a disclosed method of stimulating an effector cell mediated immune modulator response to PS-expressing tumor cells can further comprise subjecting a disclosed blood sample to centrifugation. In an aspect, a disclosed method of stimulating an effector cell mediated immune modulator response to PS-expressing tumor cells can further comprise separating the blood and/or biological sample into its component parts using, for example, centrifugation, apheresis, or any technique to the skilled person. In an aspect, a disclosed separating step can comprise generating a layer of clear fluid, a layer of red fluid, and a thin layer in between the clear fluid layer and the red fluid layer. In an aspect, a disclosed red layer can comprise red blood cells. In an aspect, a disclosed clear layer can comprise plasma. In an aspect, a disclosed thin layer in between the red layer and the clear layer can comprise the buffy coat. In an aspect, a disclosed buffy coat can comprise white blood cells and platelets. In an aspect, a disclosed method can further comprise isolating peripheral blood mononuclear cells (PMBCs) from the buffy coat. In an aspect, PMBCs can comprise lymphocytes, leukocytes, and/or monocytes. In an aspect, macrophages can be derived from monocytes. In an aspect, isolating lymphocytes, leukocytes, and/or monocytes can be done by any method and/or technique known to the skilled person (e.g., leukapheresis).

In an aspect, a disclosed method of stimulating an effector cell mediated immune modulator response to PS-expressing tumor cells can further comprise isolating resting or M0 macrophages from the buffy coat. In an aspect, a disclosed method of stimulating an effector cell mediated immune modulator response to PS-expressing tumor cells can further comprise differentiating monocytes into resting or M0 macrophages. In an aspect of a disclosed method of stimulating an effector cell mediated immune modulator response to PS-expressing tumor cells, the disclosed macrophages can be resting or M0 macrophages. In an aspect, a disclosed method of stimulating an effector cell mediated immune modulator response to PS-expressing tumor cells can further comprise polarizing the resting or M0 macrophages into a M1 phenotype or a M2 phenotype or a pro-inflammatory phenotype or an anti-inflammatory phenotype. In an aspect, a disclosed method of stimulating an effector cell mediated immune modulator response to PS-expressing tumor cells can further comprise polarizing the resting or M0 macrophages into a classically activated macrophage phenotype.

In an aspect, a therapeutically effective amount or effective dose or effective amount or therapeutically effective dosage of disclosed engineered T cells or NK cells or macrophages can be any amount that, when used alone or in combination with another therapeutic agent, can attack and destroy PS-expressing tumor cells. In an aspect, a therapeutically effective amount or effective dose or effective amount or therapeutically effective dosage of the disclosed genetically modified cells expressing a chimeric antigen receptor targeting phosphatidylserine (PS) or a pharmaceutical formulation thereof or the disclosed engineered T cells or NK cells or macrophages expressing a chimeric antigen receptor targeting phosphatidylserine (PS) or a pharmaceutical formulation thereof can be any amount that, when used alone or in combination with another therapeutic agent, can attack and destroy PS-expressing tumor cells.

In an aspect, a therapeutically effective amount of the disclosed genetically modified cells expressing a disclosed chimeric antigen receptor targeting phosphatidylserine (PS) (e.g., T cells, NK cells, and/or macrophages) can be about 1×10⁴ to about 1×10⁹ cells/kg per subject. In an aspect, a therapeutically effective amount of the disclosed genetically modified cells expressing a disclosed chimeric antigen receptor targeting phosphatidylserine (PS) (e.g., T cells, NK cells, and/or macrophages) can be about 1×10⁵ cells/kg per subject, about 1×10⁶ cells/kg per subject, about 1×10⁷ cells/kg per subject, about 1×10⁸ cells/kg per subject, or about 1×10⁹ cells/kg per subject.

In an aspect, a therapeutically effective amount or effective dose or effective amount or therapeutically effective dosage of the disclosed genetically modified cells expressing a chimeric antigen receptor targeting phosphatidylserine (PS) or a pharmaceutical formulation thereof or the disclosed engineered T cells or NK cells or macrophages expressing a chimeric antigen receptor targeting phosphatidylserine (PS) or a pharmaceutical formulation thereof can protect a subject against the onset of a disease and/or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In an aspect, administering genetically modified cells expressing a chimeric antigen receptor targeting phosphatidylserine (PS) or a pharmaceutical formulation thereof or administering engineered T cells or NK cells or macrophages expressing a chimeric antigen receptor targeting phosphatidylserine (PS) or a pharmaceutical formulation thereof can comprise systemic or direct administration. In an aspect, administering can comprise oral administration, intravenous administration, intratumoral administration, intraperitoneal administration, or any combination thereof. In an aspect, administering genetically modified cells expressing a chimeric antigen receptor targeting phosphatidylserine (PS) or a pharmaceutical formulation thereof or administering engineered T cells or NK cells or macrophages expressing a chimeric antigen receptor targeting phosphatidylserine (PS) or a pharmaceutical formulation thereof can be administered by any method of administration disclosed herein. In an aspect, genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof can be administered via multiple routes either concurrently or sequentially.

For example, in an aspect, genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof can be first administered intratumorally and then be administered intravenously. In an aspect, genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof can be first administered intratumorally and then be administered orally. A skilled clinician can determine the best route of administration for a subject at a given time.

In an aspect, a disclosed method can comprise repeating the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof.

In an aspect, a disclosed method of stimulating an effector cell mediated immune modulator response to PS-expressing tumor cells can comprise protecting the subject from metastasis. In an aspect, a disclosed method of stimulating an effector cell mediated immune modulator response to PS-expressing tumor cells can comprise reducing the risk of developing metastasis. In an aspect, a disclosed method of stimulating an effector cell mediated immune modulator response to PS-expressing tumor cells can comprise preventing or inhibiting metastasis.

In an aspect, a disclosed method of stimulating an effector cell mediated immune modulator response to PS-expressing tumor cells can effect tumor cell death.

In an aspect, a disclosed method can comprise monitoring the subject for adverse effects. In an aspect, in the absence of adverse effects, a disclosed method can comprise continuing to treat the subject. In an aspect, continuing to treat the subject can comprise continuing to administer the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof administered to the subject, changing the frequency of administration of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof, changing the duration of administration of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof, changing the route of administration of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof, or any combination thereof.

In an aspect, a disclosed method of stimulating an effector cell mediated immune modulator response to PS-expressing tumor cells can further comprise administering to the subject an immune checkpoint inhibitor (e.g., an anti-PD1 molecule). In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed method of stimulating an effector cell mediated immune modulator response to PS-expressing tumor cells can further comprise repeating the administering of the anti-PD1 molecule. In an aspect, a disclosed anti-PD1 molecule can be administered prior to, concurrent with, or after the administration of the interfering molecule.

In an aspect of a disclosed method of stimulating an effector cell mediated immune modulator response to PS-expressing tumor cells, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

For example, in an aspect, a disclosed anti-PD1 molecule can be administered about 3 months, about 2 months, or about 1 month prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof.

In an aspect, a disclosed method of stimulating an effector cell mediated immune modulator response to PS-expressing tumor cells can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method of stimulating an effector cell mediated immune modulator response to PS-expressing tumor cells can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a disclosed method of stimulating an effector cell mediated immune modulator response to PS-expressing tumor cells can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of stimulating an effector cell mediated immune modulator response to PS-expressing tumor cells can further comprise preventing or inhibiting metastasis of cancer cells. In an aspect, a disclosed method can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof)). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof).

In an aspect, a disclosed method of stimulating an effector cell mediated immune modulator response to PS-expressing tumor cells can comprise surgically resecting the tumor and/or cancer cells from the subject. In an aspect, following resecting the tumor and/or cancer cells from the subject, a disclosed method of stimulating an effector cell mediated immune modulator response to PS-expressing tumor cells can comprise continuing to administer to the subject a therapeutically effective amount of the genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof and continuing to administer to the subject a therapeutically effective amount of an anti-PD1 molecule, a disclosed anti-chemokine therapy, a disclosed chemotherapeutic agent, any disclosed therapeutic agent, or any combination thereof.

In an aspect, a disclosed method of stimulating an effector cell mediated immune modulator response to PS-expressing tumor cells can further comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of stimulating an effector cell mediated immune modulator response to PS-expressing tumor cells can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed method of stimulating an effector cell mediated immune modulator response to PS-expressing tumor cells, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect, a disclosed method of stimulating an effector cell mediated immune modulator response to PS-expressing tumor cells can further comprise administering genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof one or more times, administering an anti-PD1 molecule one or more times, administering one or more anti-cancer therapies one or more times, or administering any combination thereof one or more time.

In an aspect, preventing or inhibiting metastasis of cancer cells can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof).

In an aspect, a disclosed method of stimulating an effector cell mediated immune modulator response to PS-expressing tumor cells can comprise surgically resecting the tumor and/or cancer cells from the subject. In an aspect, following resecting the tumor and/or cancer cells from the subject, a disclosed method of stimulating an effector cell mediated immune modulator response to PS-expressing tumor cells can comprise continuing to administer to the subject a therapeutically effective amount of a disclosed pharmaceutical formulation.

In an aspect, a disclosed method of stimulating an effector cell mediated immune modulator response to PS-expressing tumor cells can further comprise diagnosing the subject as have cancer or cancerous cells.

In an aspect, a disclosed method of stimulating an effector cell mediated immune modulator response to PS-expressing tumor cells can improve and/or extend the survivability of the subject, can improve a subject''s quality of life, can increase and/or prolong a subject''s life span, or any combination thereof. In an aspect, a disclosed method of stimulating an effector cell mediated immune modulator response to PS-expressing tumor cells can stimulate an effector cell mediated immune modulator response to PS-expressing tumor cells. In an aspect, a disclosed method of stimulating an effector cell mediated immune modulator response to PS-expressing tumor cells can induce a tumor reducing immune response. In an aspect, a disclosed method of stimulating an effector cell mediated immune modulator response to PS-expressing tumor cells can induce phagocytosis of cancer cells in the subject. In an aspect, a disclosed method stimulating an effector cell mediated immune modulator response to PS-expressing tumor cells can cross-prime an anti-tumor T cell response. In an aspect, a disclosed method of stimulating an effector cell mediated immune modulator response to PS-expressing tumor cells induce a tumor eliminating immune response. In an aspect, a disclosed pharmaceutical formulation can treat cancer. In an aspect, a disclosed method stimulating an effector cell mediated immune modulator response to PS-expressing tumor cells can comprise administering to the subject a chimeric fusion receptor comprising a PS-binding domain.

### G. Kits

Disclosed herein is a kit comprising one or more of a disclosed CAR, a disclosed isolated nucleic acid molecule, a disclosed vector, a disclosed pharmaceutical formulation, a disclosed host cell, or any combination thereof. Disclosed herein is a kit comprising one or more disclosed CARs, one or more disclosed isolated nucleic acid molecules, one or more disclosed vectors, one or more disclosed pharmaceutical formulations, one or more disclosed cells, or any combination thereof.

In an aspect, a disclosed kit can comprise one or more additional active agents and/or therapeutic agents. In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to cancer or cancer cells or cancerous cells.

In an aspect, a disclosed kit can comprise at least two components constituting the kit. Together, the components constitute a functional unit for a given purpose (such as, for example, treating a subject diagnosed with or suspected of having a disease or disorder such as cancer). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on a computer readable memory device or downloaded from an internet website, or as recorded presentation. In an aspect, a kit for use in a disclosed method can comprise one or more containers holding a disclosed CAR, a disclosed isolated nucleic acid molecule, a disclosed vector, a disclosed pharmaceutical formulation, a disclosed host cell, or any combination thereof, and a label or package insert with instructions for use. In an aspect, suitable containers include, for example, bottles, vials, syringes, blister pack, etc. The containers can be formed from a variety of materials such as glass or plastic. The container can hold a disclosed CAR, a disclosed isolated nucleic acid molecule, a disclosed vector, a disclosed pharmaceutical formulation, a disclosed host cell, or any combination thereof, and can have a sterile access port (for example the container may be an intravenous solution bag or a vial having a stopper pierceable by a hypodermic injection needle). The label or package insert can indicate a disclosed CAR, a disclosed isolated nucleic acid molecule, a disclosed vector, a disclosed pharmaceutical formulation, a disclosed host cell, or any combination thereof can be used for treating, preventing, inhibiting, and/or ameliorating a disease or disorder or complications and/or symptoms associated with a disease or disorder such as cancer or metastatic cancer. A kit can comprise additional components necessary for administration such as, for example, other buffers, diluents, filters, needles, and syringes.

In an aspect, a disclosed kit can be used to treat cancer. In an aspect, a disclosed kit can be used to stimulate an effector cell mediated immune modulator response to PS-expressing tumor cells. In an aspect, a disclosed kit can be used to preventing or inhibiting metastasis of cancer cells. In an aspect, a disclosed kit can be used to risk of developing metastases. In an aspect, a disclosed kit can be used to validate the efficacy and/or toxicity of a disclosed CAR, a disclosed isolated nucleic acid molecule, a disclosed vector, a disclosed pharmaceutical formulation, a disclosed host cell, or any combination thereof.

### H. Miscellaneous

Disclosed herein is a chimeric antigen receptor (CAR) that binds phosphatidylserine (PS) on the surface of tumor cells and activates immune effector cells to eliminate tumor cells that express PS on their surface spontaneously or under stress, the CAR receptor comprising: (i) an extracellular portion that is a PS-binding domain; (ii) a transmembrane domain; and (iii) an intracellular immunostimulatory domain. In an aspect, the intracellular immunostimulatory domain comprises one or more immunostimulatory signaling domains from different immunostimulatory proteins. In an aspect, the extracellular PS-binding domain is selected from the group consisting of: (1) Human annexin A1 or its PS-binding core domain (aa41-346); (2) Human annexin A2; (3) Human annexin A3; (4) Human annexin A4; (5) Human annexin A5 (Annexin V); (6) Human annexin A6; (7) Human annexin A7; (8) Human annexin A8; (9) Human annexin A8L1; (10) Human annexin A8L2; (11) Human annexin A9; (12) Human annexin A10; (13) Human annexin A11; (14) Human annexin A13; (15) Extracellular domain of human BAI1 (brain angiogenesis inhibitor 1); (16) Human beta 2-glycoprotein I; (17) Human factor II; (18) Human factor VII; (19) Human factor IX; (20) Human factor X; (21) Human prothrombin; (22) Human growth arrest specific 6 (GAS6),; (23) Human MFG-E8 (lactaherin); (24) Extracellular domain of human RAGE; (25) Protein S; (26) Extracellular domain of STABILIN 1 & 2; (27) Extracellular domain of TIM1; (28) Extracellular domain of Tim3; (29) Extracellular domain of TIM4; (30) Phosphoatidylserine receptor (PSR); (31) Human PKCα C2 domain (aa 157-288); (32) RAGE (receptor for advanced glycation end products); (33) Human synaptotagmin (Syt1) C2A domain (aa141-266); (34) Stabilin-1; (35) Stabilin-2; and combinations thereof.

In an aspect, the extracellular domain comprises one or more chain variable domains (scFv). In an aspect, the chain variable domains (scFv) comprises a PS-binding antibody selected from the group consisting of: (1) Bavituximab; (2) PS binding antibody PGN632 (as published by Moody et al, JEM, 2010, 207:763-776); (3) PS binding antibody P1 (as published by Moody et al, JEM, 2010, 207:763-776); (4) PS binding antibody IS4 (as published by Moody et al, JEM, 2010, 207:763-776); (5) PS binding antibody CL1 (as published by Moody et al, JEM, 2010, 207:763-776); and combinations thereof. In an aspect, the extracellular domain further includes a spacer domain between the extracellular PS-binding domain and the transmembrane domain.

In an aspect, the spacer comprises: (i) an immunoglobulin hinge region; and (ii) an extracellular region of type 1 membrane proteins or the whole or part of immunoglobulin constant region. In an aspect, the extracellular spacer domain comprises the hinge regions, or any portion thereof, from the hinge regions from the group consisting of (1) CD8a; (2) CD28; (3) IgG1; (4) IgG2; (5) IgG3; (6) IgG4; (7) IgA; (8) IgD; and combinations thereof.

In an aspect, the transmembrane domain comprises a transmembrane domain of a transmembrane protein selected from the group consisting of: (1) CD2; (2) CD3γ; (3) CD3ε; (4) CD3δ; (5) CD3ξ; (6) CD4; (7) CD8; (8) CD25; (9) CD27; (10) CD28; (11) CD40; (12) CD79A; (13) CD79B; (14) CD80; (15) CD86; (16) CD95 (FAS); (17) CD134 (OX40); (18) CD137 (4-1BB); (19) CD278 (ICOS); (20) TCRα; (21) TCRβ; and combinations thereof.

In an aspect, the intracellular signaling domain comprises a single ITAM signaling domain selected from the group consisting of the signaling domains from the following immune signaling proteins: (1) CD3γ; (2) CD3ξ; (3) CD3ε; (4) CD3δ; (5) CD3ξ; (6) CD5; (7) CD22; (8) CD79a; (9) CD278 (ICOS); and combinations thereof. In an aspect, the CAR further comprises a co-stimulatory domain from the signaling domains of an immune-signaling molecule that is selected from the group consisting of: (1) CD27; (2) CD28; (3) 4-1BB; (4) OX40; (5) CD30; (6) CD40; (7) PD-1; (8) ICOS; (9) LFA-1; (10) CD2; (11) CD7; (12) LIGHT; (13) NKG2C; (14) B7-H3; and combinations thereof. In an aspect, the CAR comprises one ITAM signaling domain, a first co-stimulatory domain, and a second co-stimulatory domain. In an aspect, the CAR comprises a first generation, a second generation, or a third generation CAR. In an aspect, the extracellular membrane moiety comprises: (i) an Annexin V; (ii) an CD8a hinge region; (iii) a CD8a transmembrane domain: (iv) an intracellular domain that comprises a 4-1BB and CD3ξ; and (v) a truncated EGFR co-expressed from a P2A linker.

Disclosed herein is a CAR comprising the structure selected from the group consisting of: (i) A5-BBz-EGFRt; (ii) A5-28z-EGFRt; (iii) A5-28BBz-EGFRt; and any fragment or portions thereof. Disclosed herein is a CAR comprising SEQ ID NO: 1 and any fragment or portion thereof. Disclosed herein is a CAR comprising SEQ ID NO: 2 and any fragment or portion thereof. Disclosed herein is a CAR comprising SEQ ID NO: 3 and any fragment or portion thereof.

In an aspect, the CARs provided herein are expressed in a vector/vector system. In an aspect, the vector/vector system is selected from the group consisting of: (1) lentivirus; (2) retrovirus; (3) transposon-based plasmids such as sleeping beauty and piggyback that were introduced through electroporation; (4) mRNA encoding the CAR receptor that are electroporated into T cells; (5) nanoparticles targeted at T cells with mRNA or transposon-based plasmids; and combinations thereof. In an aspect, the CARs are expressed on an immune effector cell. In an aspect, the cell is selected from the group consisting of T cells, NK cells, or macrophages for cancer treatment. In an aspect, the cancer cells to be targeted are those the express PS cells on their surface but remain alive, which can occur in cytotoxic therapy (chemotherapy or radiotherapy)-treated or -untreated cancer cells.

Disclosed herein is a pharmaceutical composition comprising a CAR as in any of the preceding claims and a pharmaceutically acceptable excipient, diluent, and/or carrier.

Disclosed herein is a method for treating solid and/or liquid tumors in a subject, the method comprising administering to the subject a therapeutically effective amount of a CAR as in any of the preceding claims such that the solid and/or liquid tumors are treated. In an aspect, the CARs are expressed on an immune effector cell. In an aspect, the immune effector cell is selected from the group consisting of T cell, NK cell, macrophages and combinations thereof. In an aspect, tumor comprises a liquid tumor selected from the group consisting of lymphoma and leukemia. In an aspect, the tumor cells comprise metastatic cancer cells. In an aspect, the metastatic cancer cells are found the blood, lymphatic system, and/or peritoneal cavity.

| SEQ ID | | |
|---|---|---|
| NO. | Description of Sequence | Type |
| 1 | Annexin V-CAR version 1: A5-BBz-EGFRt | synthetic |
| 2 | Annexin V-CAR version 2: A5-28z-EGFRt | synthetic |
| 3 | Annexin V-CAR version 3: A5-28BBz-EGFRt | synthetic |
| 4 | P2A//signal//tEGFR | synthetic |
| 5 | Annexin V-CAR version 1: A5-BBz-EGFRt | synthetic |
| | (w/o EGFR) | |
| 6 | Annexin V-CAR version 2: A5-28z-EGFRt | synthetic |
| | (w/o EGFR) | |
| 7 | Annexin V-CAR version 3: A5-28BBz-EGFRt | synthetic |
| | (w/o EGFR) | |
| 8 | CD8 signal peptide | synthetic |
| 9 | CD8α hinge domain | synthetic |
| 10 | CD8α transmembrane domain | synthetic |
| 11 | CD28 intracellular domain | synthetic |
| 12 | CD28a transmembrane domain | synthetic |
| 13 | 4-1BB intracellular domain | synthetic |
| 14 | CD3z intracellular domain | synthetic |
| 15 | P2A Cleaving Peptide | synthetic |
| 16 | Signal peptide (SP) | synthetic |
| 17 | EGFR | human protein |
| 18 | Truncated EGFR | human protein |
| 19 | ANXA1 | human protein |
| 20 | ANXA1 (PS Binding Domain of 41-346) | human protein |
| 21 | ANXA2 | human protein |
| 22 | ANXA3 | human protein |
| 23 | ANXA4 | human protein |
| 24 | ANXA5 | human protein |
| 25 | ANXA6 | human protein |
| 26 | ANXA7 | human protein |
| 27 | ANXA8 | human protein |
| 28 | ANXA8L1 | human protein |
| 29 | ANXA9 | human protein |
| 30 | ANXA10 | human protein |
| 31 | ANXA11 | human protein |
| 32 | ANXA13 | human protein |
| 33 | ADGRB1 (BAI1) | human protein |
| 34 | ADGRB1 (BAI) (extracellular domain 31-948) | human protein |
| 35 | Apolipoprotein H (Beta-2-Glycoprotein I) | human protein |
| 36 | Coagulation Factor II | human protein |
| 37 | Coagulation Factor VII | human protein |
| 38 | Coagulation Factor IX | human protein |
| 39 | Coagulation Factor X | human protein |
| 40 | Growth Arrest Specific 6 | human protein |
| 41 | Milk Fat Globule EGF and Factor V/VIII | human protein |
| | Domain Containing (MFG-E8) (Lactadherin) | |
| 42 | AGER/RAGE | human protein |
| 43 | AGER/RAGE (ExD 23-342) | human protein |
| 44 | Protein S | human protein |
| 45 | Stabilin 1 | human protein |
| 46 | Stabilin 1 (ExD 26-2478) | human protein |
| 47 | Stabilin 2 | human protein |
| 48 | Stabilin 2 (ExD 20-2458) | human protein |
| 49 | Hepatitis A Virus Cellular Receptor 1/TIM1 | human protein |
| 50 | Hepatitis A Virus Cellular Receptor 1/TIM1 | human protein |
| | (ExD 21-295) | |
| 51 | Hepatitis A Virus Cellular Receptor 2/TIM3 | human protein |
| 52 | Hepatitis A Virus Cellular Receptor 2/TIM3 | human protein |
| | (Ex 22-202) | |
| 53 | T Cell Immunoglobulin And Mucin Domain | human protein |
| | Containing (TIMD4) | |
| 54 | T Cell Immunoglobulin And Mucin Domain | human protein |
| | Containing/TIMD4 (Extracellular Domain | |
| | 25-314) | |
| 55 | Protein Kinase C Alpha | human protein |
| 56 | Protein Kinase C Alpha (C2 domain 158-275) | human protein |
| 57 | Synaptotagmin (SYT1) | human protein |
| 58 | Synaptotagmin (C2A domain 142-261) | human protein |
| 59 | Jumonji Domain Containing 6, Arginine | human protein |
| | Demethylase and Lysine Hydrolase (JMJD6) | |
| 60 | Annexin A1 | human genomic |
| 61 | Annexin A2 | human genomic |
| 62 | Annexin A3 | human genomic |
| 63 | Annexin A4 | human genomic |
| 64 | Annexin A5 | human genomic |
| 65 | Annexin A6 | human genomic |
| 66 | Annexin A7 | human genomic |
| 67 | Annexin A8 (human genomic)v Annexin A8 | human genomic |
| 68 | Annexin A8L1 | human genomic |
| 69 | Annexin A9 | human genomic |
| 70 | Annexin A10 | human genomic |
| 71 | Annexin A11 | human genomic |
| 72 | Annexin A13 | human genomic |
| 73 | scFV of Bavitixumab | synthetic |
| 74 | Annexin A1 (mRNA - NM_000700.3) | human |
| 75 | Annexin A2 (mRNA transcript 1 - NM_001002858.3) | human |
| 76 | Annexin A2 (mRNA transcript 2 - NM_001002857.2) | human |
| 77 | Annexin A3 (mRNA transcript - NM_005139.3) | human |
| 78 | Annexin A4 (mRNA transcript 1 - NM_001320698.2) | human |
| 79 | Annexin A4 (mRNA transcript 2 - NM_001153.5) | human |
| 80 | Annexin A4 (mRNA transcript 3 - NM_001320700.2) | human |
| 81 | Annexin A4 (mRNA transcript 4 - NM_001320702.2) | human |
| 82 | Annexin A5 (mRNA - NM_001154.4) | human |
| 83 | Annexin A6 (mRNA transcript 1 - NM_001155.5) | human |
| 84 | Annexin A6 (mRNA transcript 2 - NM_001193544.2) | human |
| 85 | Annexin A6 (mRNA transcript 3 - NM_001363114.2) | human |
| 86 | Annexin A7 (mRNA transcript 1 - NM_001156.5) | human |
| 87 | Annexin A7 (mRNA transcript 2 - NM_0044.4) | human |
| 88 | Annexin A7 (mRNA transcript 3 - NM_001320880.2) | human |
| 89 | Annexin A7 (mRNA transcript 4 - NM_001320879.2) | human |
| 90 | Annexin A8 (mRNA transcript 1 - NM_001271702.2) | human |
| 91 | Annexin A8 (mRNA transcript 2 - NM_001040084.3) | human |
| 92 | Annexin A8 (mRNA transcript 3 - NM_0012717.2) | human |
| 93 | Annexin A8L1 (mRNA transcript 1 - NM_001098845.3) | human |
| 94 | Annexin A8L1 (mRNA transcript 2 - NM_001278924.2) | human |
| 95 | Annexin A8L1 (mRNA transcript 3 - NM_001278923.2 | human |
| 96 | Annexin A9 (mRNA transcript - NM_0568.3) | human |
| 97 | Annexin 10 (mRNA transcript 1 - NM_007193.5) | human |
| 98 | Annexin A11 (mRNA transcript a - NM_001157.3) | human |
| 99 | Annexin A11 (mRNA transcript b - NM_001278409.2) | human |
| 100 | Annexin A13 (mRNA transcript a - NM_004306.4) | human |
| 101 | Annexin A13 (mRNA transcript b - NM_0010954.3) | human |
| 102 | P2A Cleaving Peptide with Optional Linker | synthetic |
| 103 | T2A Cleaving Peptide | synthetic |
| 104 | T2A Cleaving Peptide with Optional Linker | synthetic |
| 105 | E2A Cleaving Peptide | synthetic |
| 106 | E2A Cleaving Peptide with Optional Linker | synthetic |
| 107 | F2A Cleaving Peptide | synthetic |
| 108 | F2A Cleaving Peptide with Optional Linker | synthetic |

### EXAMPLES

Most of the current paradigms for cancer therapy assume that PS-expressing tumor cells, either from exposure to internal or external stressors, are destined to die from apoptosis. But data provided herein showed that some of these PS-expressing tumor cells survived. As detailed herein, the compositions and methods described herein can significantly enhance the success rate of conventional cytotoxic cancer therapy. The Examples that follow are illustrative of specific aspects of the invention, and various uses thereof. They set forth for explanatory purposes only and are not to be taken as limiting the invention.

### Example 1

### Examination of Survival of Phosphatidylserine-Positive Tumor Cells

To determine whether cells expressing high-level of PS expression on the cell surface were destined to die, the viability of c1498 murine AML (Acute Myeloid Leukemia) cells treated with cytarabine (part of a classical chemotherapy regimen for human AML) was examined.

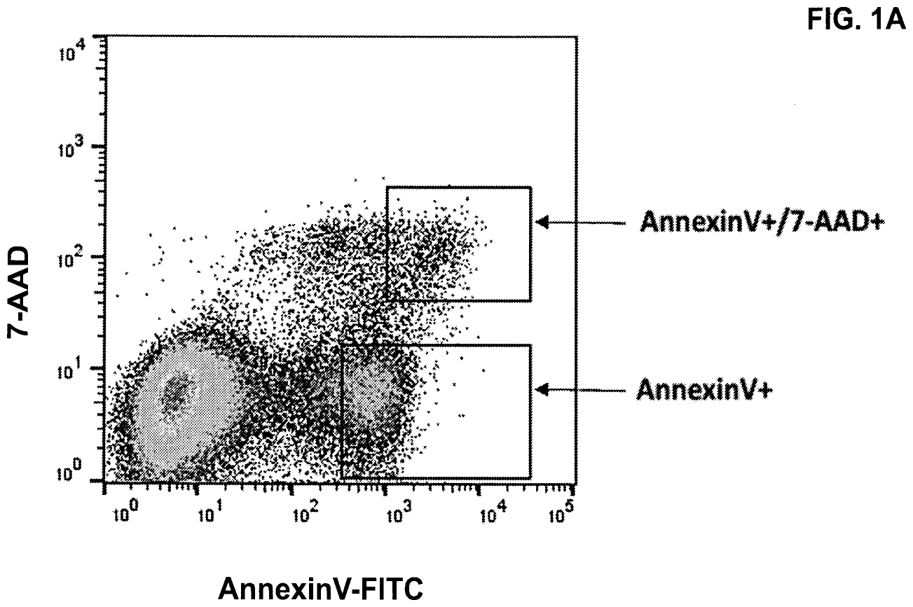

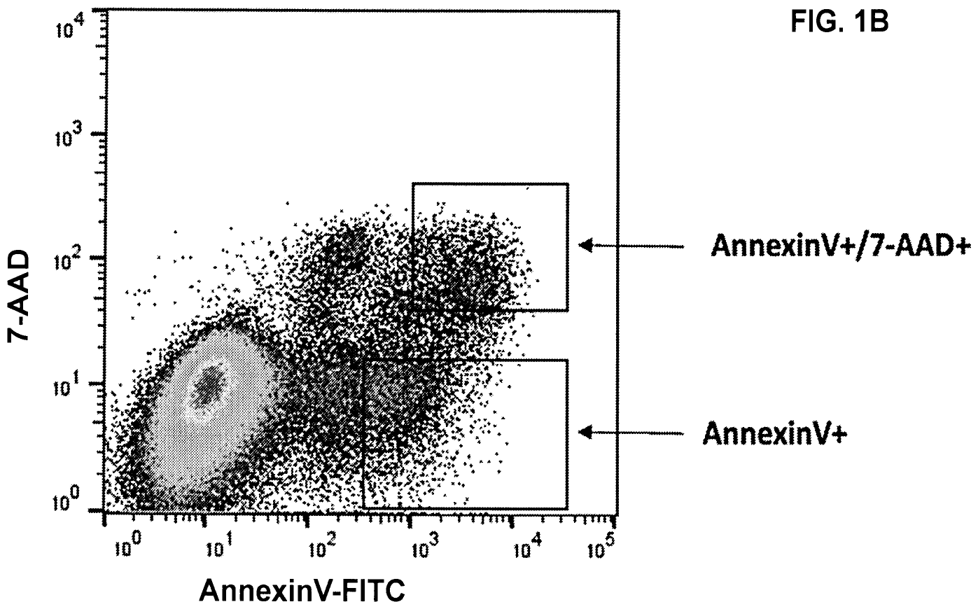

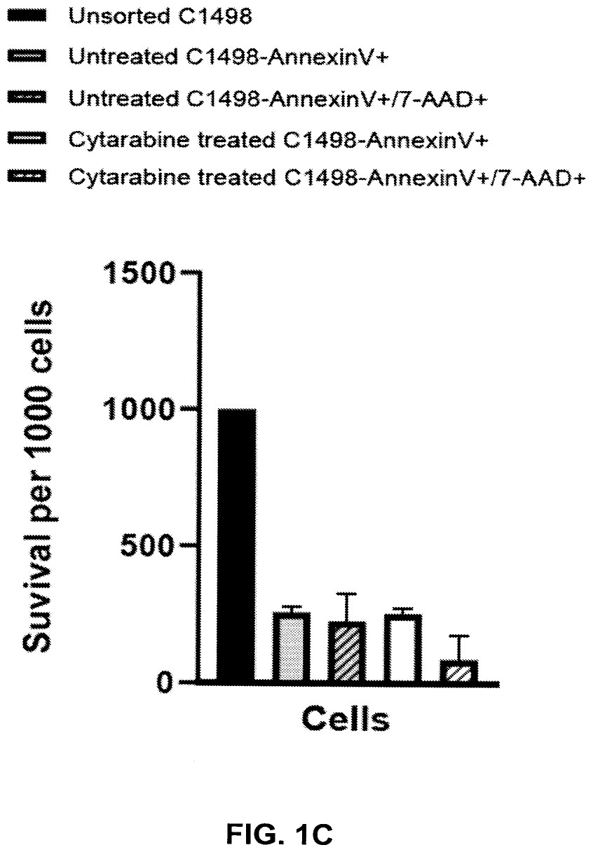

Specifically, whether PS-expressing acute myelocytic leukemia (AML) cells survived was examined. FACS was used to identify and sort c1498 cells treated with 1 μM cytarabine (for 24 hrs) and with high PS-expression. The sorted cells were then seeded into 6-well dishes and the ability to survive and proliferate was measured. FIG. 1A-FIG. 1B). The data indicated the following: (i) about 22-25% of untreated c1498 cells that were either PS+7AAD-(7AAD is a fluorescent dye used to detect membrane integrity), early apoptotic cells, or PS+7AAD+, late apoptotic cells, remained alive and could proliferate in a robust manner (FIG. 1C), and (ii) about 25% or 8% of cytarabine-treated PS+7AAD-c1498 cells or PS+7AAD+c1498 cells, respectively, remained viable and could proliferate (FIG. 1C). These results indicated that a substantial proportion of PS+ cells survived the initiation of the apoptotic process, which is different from the prevailing view that PS+ cells are destined to die.

### Example 2

### Annexin V-CAR Transduced T-Cell Mediated Killing of Human AML Cells Chemotherapy Treatment In Vitro

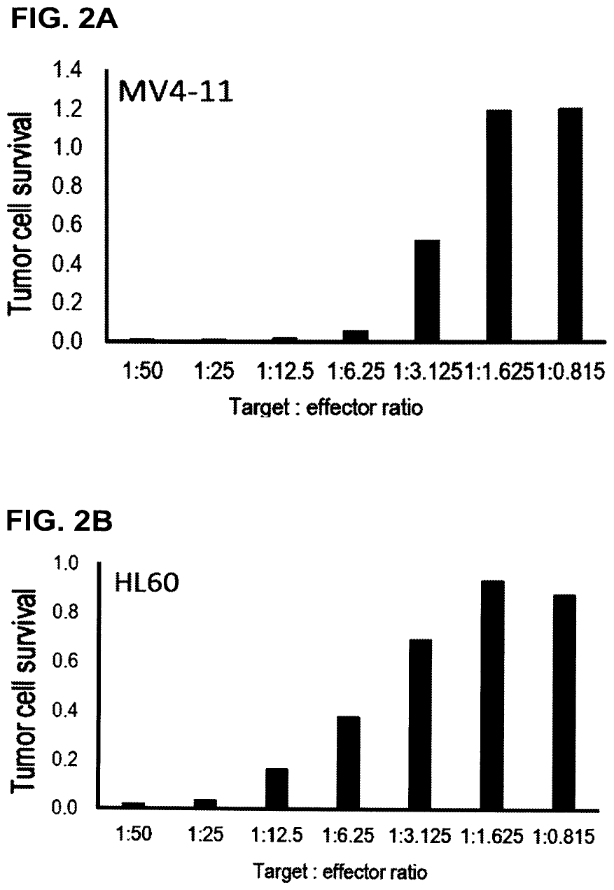

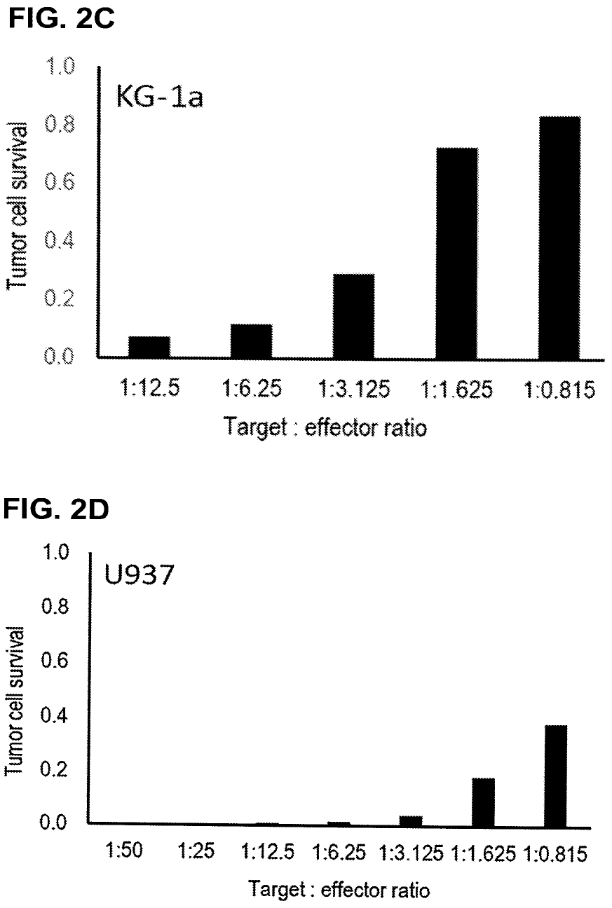

To evaluate PS-targeting CAR-T cells'' abilities to kill cancer cells, Annexin V-CAR (Annexin V-BBz)-transduced T cells were evaluated. CAR-T cells were co-cultured with 4 different AML cells: MV4-11, HL60, KG-1a, and U937. These cells were genetically transduced with the firefly luciferase (Fluc) gene. The efficacy of the CAR-T cells was evaluated by measuring the relative numbers of the tumor cells after 24 hrs of co-culture, through measuring Fluc activities of the surviving cells by administering luciferin, a chemical substrate of Fluc. In all 4 cells, the CAR-T cells had potent abilities to kill the tumor cells, especially at low tumor: T cell ratios (FIG. 2).

### Example 3

### Annexin V-CAR Transduced T-Cell Mediated Killing of Human AML Cells Chemotherapy Treatment In Vivo

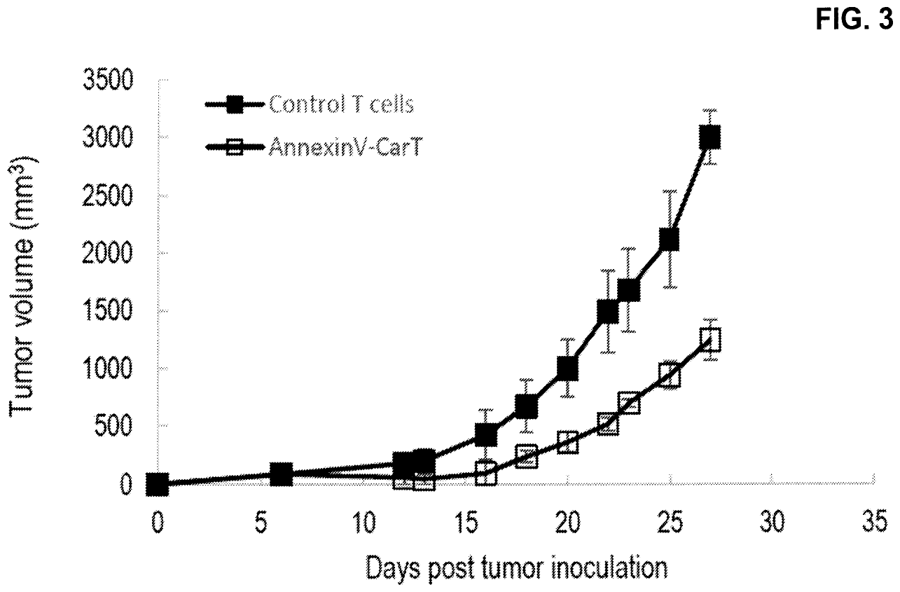

The ability of the annexin V-CAR transduced T cells to suppress tumor growth in vivo was evaluated. Subcutaneous KG1a AML tumors were established by injecting 2×10⁶ KG-la cells subcutaneously. Then, 6 days later, 1×10⁷ Annexin V-CAR transduced T cells were injected intravenously. Tumor dimensions were then measured by using a caliper. The data indicated that Annexin V-CAR transduced cells significantly suppressed KG-1a tumor growth (FIG. 3). Tumor volume were calculated as: V (tumor)=(π/6)*(L)*(S) 2, V=tumor volume, π=3.14, L: longest dimension of the tumor, and S=shortest dimension of the tumor.

### Summary of Specific Example

As demonstrated herein, genetically modified immune effector cells (T, NK, NKT, or macrophages) expressing a PS-targeting chimeric antigen receptor can eliminate PS-expressing tumor cells. The examples provided herein demonstrated that PS-expressing tumor cells, occurring either spontaneously or after exposure to cytotoxic chemotherapeutic agents, could survive and proliferate, indicating that these cells are important therapeutic targets that must be eliminated for cancer therapy to be successful. Because the art has previously discounted the significance of PS-expressing tumor cells, there are no current therapeutics that can effectively target these tumor cells. The examples provided herein conclusively demonstrate that the disclosed CAR-modified T cells (or NK cells or macrophages) effectively killed PS⁺ tumor cells both in vitro and in vivo. Furthermore, because cell surface PS expression is a dynamic process for treated tumor cells, PS-negative tumor cells likely become PS-positive in a stochastic manner. For this reason, the disclosed PS-targeting CAR-T cells can kill a significant number of PS-negative cancer cells.

What is claimed is:

1. A chimeric antigen receptor (CAR), comprising:
an extracellular domain comprising a phosphatidylserine (PS) antigen binding domain, a transmembrane domain; and
an intracellular domain comprising one or more immunostimulatory domains.

2. The CAR of claim 1, wherein the CAR comprises the sequence set forth in any one of SEQ ID NO: 01-SEQ ID NO:03.

3. The CAR of claim 1, wherein the CAR comprises the sequence set forth in any one of SEQ ID NO: 05-SEQ ID NO:07.

4. The CAR of claim 1, wherein the extracellular domain comprises a signal peptide.

5. The CAR of claim 2, wherein the signal peptide comprises a CD8 signal peptide, and wherein the CD8 signal peptide comprises the sequence set forth in SEQ ID NO:08.

6. The CAR of claim 1, wherein the PS binding domain comprises Annexin A1 or the PS-binding core domain, Annexin A2, Annexin A3, Annexin A4, Annexin A5, Annexin A6, Annexin A7, Annexin A8, Annexin A8 Like 1, Annexin A9, Annexin A10, Annexin A11, Annexin A13, Adhesion G Protein Coupled Receptor B1 or the extracellular domain thereof, Apolipoprotein H, Coagulation Factor II, Coagulation Factor VII, Coagulation Factor IX, Coagulation Factor X, Growth Arrest Specific 6, Milk Fat Globule EGF And Factor V/VIII Domain Containing, Advanced Glycosylation End-Product Specific Receptor or the extracellular domain thereof, Protein S, Hepatitis A Virus Cellular Receptor 1 or the extracellular domain thereof, Hepatitis A Virus Cellular Receptor 2 or the extracellular domain thereof, T Cell Immunoglobulin and Mucin Domain Containing or the extracellular domain thereof, Protein Kinase C alpha or the C2 domain thereof, Synaptotagmin (Syt1) or the C2A domain thereof, Stabilin-1 or the extracellular domain thereof, or Stabilin-2 or the extracellular domain thereof.

7. The CAR of claim 1, wherein the PS binding domain comprises the single-chain variable domain of bavituximab, PGN632, P1, IS4, or CL1.

8. The CAR of claim 1, further comprising a spacer domain between the extracellular domain and the transmembrane domain.

9. The CAR of claim 8, wherein the spacer domain comprises a hinge region.

10. The CAR of claim 9, wherein the hinge range comprises the hinge region of CD8a, CD28, IgG1, IgG2, IgG3, IgG4, IgA, or IgD.

11. The CAR of claim 10, wherein the hinge domain comprises a CD8a hinge domain, and wherein the CD8a hinge domain comprises the sequence set forth in SEQ ID NO:09.

12. The CAR of claim 1, wherein the transmembrane domain comprises the transmembrane domain of CD2, CD3γ, CD3ε, CD3δ, CD3ξ, CD4, CD8, CD25, CD27, CD28, CD40, CD79A, CD79B, CD79B, CD80, CD86, CD95 (FAS), CD134 (OX40), CD137 (4-1BB), CD278 (ICOS), TCRα, TCRβ, or any combination thereof.

13. The CAR of claim 12, wherein the transmembrane domain comprise the transmembrane domain of CD28, and wherein the CD28 transmembrane domain comprise the sequence set forth in SEQ ID NO:12.

14. The CAR of claim 1, wherein the intracellular domain comprises one or more immunoreceptor tyrosine-based activation domains (ITAMs).

15. The CAR of claim 14, wherein the ITAM comprises the signaling domain of CD3γ, CD3ξ, CD3ε, CD3δ, CD3ξ, CD5, CD22, CD79a, CD278 (ICOS), or any combination thereof.

16. The CAR of claim 1, wherein the intracellular domain comprises one or more co-stimulatory domains.

17. The CAR of claim 16, wherein the one or more co-stimulatory domains comprise the signaling domain of CD27, CD28, 4-1BB, OX40, CD30, CD40, PD-1, ICOS, LFA-1, CD2, CD7 LIGHT, NKG2C, B7-H3, or any combination thereof.

18. The CAR of claim 1, wherein the intracellular domain comprises an ITAM and one or more co-stimulatory domains, wherein the ITAM comprises the signaling domain of CD3γ, CD3ξ, CD3ε, CD3δ, CD3ξ, CD5, CD22, CD79a, CD278 (ICOS), or any combination thereof, and wherein the co-stimulatory domains comprise the signaling domain of CD27, CD28, 4-1BB, OX40, CD30, CD40, PD-1, ICOS, LFA-1, CD2, CD7 LIGHT, NKG2C, B7-H3, or any combination thereof.

19. The CAR of claim 1, wherein the intracellular domain further comprises a self-cleaving peptide, a signal peptide, and a truncated EGFR domain, or any combination thereof.

20. An isolated nucleic acid molecule encoding the CAR of any preceding claim.

21. A recombinant vector comprising the isolated nucleic acid molecule of claim 20.

22. An immune cell transformed by the recombinant vector of claim 21.

23. A pharmaceutical formulation comprising the isolated nucleic acid of claim 20, the recombinant vector of claim 21, and/or the transformed immune cell of claim 22; and one or more pharmaceutically acceptable carriers.

24. A method of treating cancer, the method comprising: administering to a subject in need thereof a therapeutically effective amount of the immune cells of claim 22 and/or the pharmaceutical formulation of claim 23, wherein, following administration, an effector cell mediated immune modulator response to PS-expressing tumor cells is stimulated.

25. The method of claim 24, wherein administering comprises oral administration, intravenous administration, intratumoral administration, intraperitoneal administration, or any combination thereof.

26. The method of claim 24, further comprising monitoring the subject for adverse effects.

27. The method of claim 26, wherein in the absence of adverse effects, the method further comprises continuing to administering to the subject the immune cells and/or the pharmaceutical formulation.

28. The method of claim 26, wherein in the presence of adverse effects, the method further comprises modifying one or more steps of the method.

29. The method of claim 24, further comprising administering to the subject one or more additional anti-cancer therapies.

30. The method of claim 29, wherein the one or more anti-cancer therapies comprises endocrine therapy, radiotherapy, hormone therapy, gene therapy, thermal therapy, ultrasound therapy, or any combination thereof.

31. The method of claim 29, wherein anti-cancer therapies comprise one or more chemotherapeutic agents.

32. The method of claim 24, wherein the subject has been diagnosed with ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

33. The method of claim 24, wherein the subject is protected from metastases, wherein the subject's risk of developing metastasis is reduced, and/or wherein the subject''s cancer is treated.
